SEQUENCE LISTING (79 sequences)

SEQ ID NO: 1 - protein, 376 aa, Artificial Sequence
  variable sequence of glycosyl transferase
  UNSURE 1..20: Xaa = any amino acid
  VARIANT 21: Lys = Arg
  UNSURE 26..27: Xaa = any amino acid
  UNSURE 29: Xaa = any amino acid
  VARIANT 34: Asn = Ser
  UNSURE 38: Xaa = any amino acid
  VARIANT 39: Leu = Ile
  UNSURE 41..46: Xaa = any amino acid
  UNSURE 48: Xaa = any amino acid
  VARIANT 53: Tyr = Phe
  UNSURE 54..84: Xaa = any amino acid
  VARIANT 85: Phe = Tyr or Leu
  VARIANT 87: Glu = Asp
  UNSURE 89..99: Xaa = any amino acid
  UNSURE 102: Xaa = Ala, Ile, Leu, Met, Phe, Pro, Trp or Val
  UNSURE 103..104: Xaa = any amino acid
  UNSURE 105: Xaa = Ala, Ile, Leu, Met, Phe, Pro, Trp or Val
  UNSURE 107..108: Xaa = any amino acid
  UNSURE 110..111: Xaa = any amino acid
  VARIANT 113: Tyr = Phe
  UNSURE 114: Xaa = Ala, Ile, Leu, Met, Phe, Pro, Trp or Val
  UNSURE 115: Xaa = any amino acid
  UNSURE 117..123: Xaa = any amino acid
  VARIANT 124: Phe = Trp
  UNSURE 127: Xaa = any amino acid
  UNSURE 128..130: Xaa = Ala, Ile, Leu, Met, Phe, Pro, Trp or Val
  UNSURE 131: Xaa = any amino acid
  VARIANT 132: Asp = Glu
  UNSURE 133..134: Xaa = any amino acid
  UNSURE 136..139: Xaa = any amino acid
  UNSURE 141..155: Xaa = any amino acid
  UNSURE 158: Xaa = any amino acid
  UNSURE 160: Xaa = Asn, Cys, Gln, Gly, Ser, Thr or Tyr
  UNSURE 161..163: Xaa = any amino acid
  VARIANT 165: Pro = Ala
  UNSURE 167: Xaa = any amino acid
  UNSURE 169: Xaa = any amino acid
  UNSURE 171..172: Xaa = any amino acid
  UNSURE 174..178: Xaa = any amino acid
  VARIANT 180: Lys = Arg
  UNSURE 181..229: Xaa = any amino acid
  UNSURE 232: Xaa = any amino acid
  VARIANT 233: Gly = Cys
  UNSURE 234: Xaa = any amino acid
  VARIANT 235: Pro = Lys
  VARIANT 238: Glu = Asp
  UNSURE 240: Xaa = any amino acid
  UNSURE 242..280: Xaa = any amino acid
  UNSURE 285: Xaa = Ala, Ile, Leu, Met, Phe, Pro, Trp or Val
  VARIANT 287: Lys = Arg
  UNSURE 288..290: Xaa = any amino acid
  UNSURE 292..294: Xaa = Ala, Ile, Leu, Met, Phe, Pro, Trp or Val
  VARIANT 301: Arg = Lys
  UNSURE 302..305: Xaa = any amino acid
  UNSURE 308..309: Xaa = Ala, Ile, Leu, Met, Phe, Pro, Trp or Val
  UNSURE 314..329: Xaa = any amino acid
  VARIANT 331: Glu = Asp
  UNSURE 337..338: Xaa = any amino acid
  VARIANT 339: Val = Ile
  UNSURE 342..343: Xaa = any amino acid
  VARIANT 346: Tyr = Phe
  VARIANT 347: Ile = Val
  VARIANT 348: Thr = Ser
  VARIANT 352: Tyr = Phe
  VARIANT 356: Met = Leu
  UNSURE 358: Xaa = any amino acid
  UNSURE 360: Xaa = any amino acid
  VARIANT 361: Asn = His
  UNSURE 362: Xaa = any amino acid
  UNSURE 365: Xaa = Ala, Ile, Leu, Met, Phe, Pro, Trp or Val
  UNSURE 367: Xaa = any amino acid
  UNSURE 370: Xaa = Ala, Ile, Leu, Met, Phe, Pro, Trp or Val
XXXXXXXXXXXXXXXXXXXXKILFAXXPXDGHFNPLTXLAXXXXXXGXDVRWYXXXXXXXXXXXXXXXXXXXXXXXXXXXXXXXFPERXXXXXXXXXXXFDXXXXFXXRXXEYXXDXXXXXXXFPFXXXXXDXXFXXXXFXXXXXXXXXXXXXXXPLXEXXXXLPPXGXGXXPXXXXXGKXXXXXXXXXXXXXXXXXXXXXXXXXXXXXXXXXXXXXXXXXXXXXXXXXLQXGXPGFEYXRXXXXXXXXXXXXXXXXXXXXXXXXXXXXXXXXXXXXXXXTQGTXEKXXXKXXXPTLEAFRXXXXLVXXTTGGXXXXXXXXXXXXXXXXIEDFIPFXXVMPXXDVYITNGGYGGVMLXIXNXLPXVXAGXHEGKNE

SEQ ID NO: 2 - DNA, 1380 bp, Artificial Sequence
  variable sequence of glycosyl transferase
  unsure 1..60: /replace=“a, t, g or c”
  variation 61..63: /replace=“mgr”
  unsure 75..81: /replace=“a, t, g, or c”
  unsure 84..87: /replace=“a, t, g or c”
  unsure 93: /replace=“a, t, g or c”
  variation 100..102: /note=“tcn - n can be any of t, c, g or a” /replace=“agy” /replace=“tcn”
  unsure 105: /replace=“a, t, g or c”
  unsure 111..114: /replace=“t, g, a or c”
  variation 115..117: /replace=“ath”
  unsure 120..138: /replace=“a, t, g or c”
  unsure 141: /replace=“a, t, g or c”
  unsure 150: /replace=“a, t, g or c”
  variation 157..159: /replace=“tty”
  unsure 160..207: /replace=“a, t, g or c”
  variation 208..210: /replace=“tay” /replace=“ytr”
  unsure 213: /replace=“a, t, g or c”
  variation 214..216: /replace=“gay”
  unsure 220..297: /replace=“a, t, g or c”
  unsure 304..315: /replace=“a, t, g or c”
  unsure 319..324: /replace=“a, t, g or c”
  unsure 329..333: /replace=“a, t, g or c”
  variation 337..339: /replace=“tty”
  unsure 340..345: /replace=“a, t, g or c”
  unsure 349..369: /replace=“a, t, g or c”
  variation 370..372: /replace=“tgg”
  unsure 375: /replace=“a, t, g or c”
  unsure 379..393: /replace=“a, t, g or c”
  variation 394..396: /replace=“gar”
  unsure 397..402: /replace=“a, t, g or c”
  unsure 406..417: /replace=“a, t, g or c”
  unsure 421..465: /replace=“a, t, g or c”
  unsure 468: /replace=“a, t, g or c”
  unsure 472..474: /replace=“a, t, g or c”
  unsure 481..489: /replace=“a, t, g or c”
  variation 493..495: /note=“n at position 495 = a, t, g or c” /replace=“gcn”
  unsure 498..501: /replace=“a, t, g or c”
  unsure 504..507: /replace=“a, t, g or c”
  unsure 510..516: /replace=“a, t, g or c”
  unsure 519..534: /replace=“a, t, g or c”
  unsure 537: /replace=“a, t, g or c”
  variation 538..540: /replace=“mgr”
  unsure 541..687: /replace=“a, t, g or c”
  unsure 694..696: /replace=“a, t, g or c”
  variation 697..699: /note=“n at position 699 = a, t, g or c” /replace=“tgy”
  unsure 700..702: /replace=“a, t, g or c”
  variation 703..705: /note=“n at position 705 = a, t, g or c” /replace=“aar”
  unsure 708: /replace=“a, t, g or c”
  variation 712..714: /replace=“gay”
  unsure 718..720: /replace=“a, t, g or c”
  unsure 724..840: /replace=“a, t, g or c”
  unsure 843: /replace=“a, t, g or c”
  unsure 849: /replace=“a, t, g or c”
  unsure 852..855: /replace=“a, t, g or c”
  unsure 862..870: /replace=“a, t, g or c”
  variation 871..873: /replace=“mgr”
  unsure 874..882: /replace=“a, t, g or c”
  unsure 885: /replace=“a, t, g or c”
  unsure 888: /replace=“a, t, g or c”
  unsure 897: /replace=“a, t, g or c”
  variation 901..903: /replace=“aar”
  unsure 904..915: /replace=“a, t, g or c”
  unsure 921..927: /replace=“a, t, g or c”
  unsure 930: /replace=“a, t, g or c”
  unsure 933: /replace=“a, t, g or c”
  unsure 936: /replace=“a, t, g or c”
  unsure 939..987: /replace=“a, t, g or c”
  variation 991..993: /replace=“gay”
  unsure 999: /replace=“a, t, g or c”
  unsure 1005: /replace=“a, t, g or c”
  unsure 1008..1014: /replace=“a, t, g or c”
  variation 1015..1017: /note=“n at position 1017 = a,t, g or c” /replace=“ath”
  unsure 1023..1029: /replace=“a, t, g or c”
  unsure 1035: /replace=“a, t, g or c”
  variation 1036..1038: /replace=“tty”
  variation 1039..1041: /note=“n on position 1041 = a, t, g or c” /replace=“gtn”
  variation 1042..1044: /note=“n on position 1043 = a, t, g or c” /replace=“tcn” /replace=“agy”
  unsure 1050: /replace=“a, t, g or c”
  unsure 1053: /replace=“a, t, g or c”
  variation 1054..1056: /replace=“tty”
  unsure 1059: /replace=“a, t, g or c”
  unsure 1062: /replace=“a, t, g or c”
  unsure 1065: /replace=“a, t, g or c”
  variation 1066..1067: /replace=“ytr”
  unsure 1072..1074: /replace=“a, t, g or c”
  unsure 1078..1080: /replace=“a, t, g or c”
  variation 1081..1083: /replace=“cay”
  unsure 1084..1086: /replace=“a, t, g or c”
  unsure 1092..1095: /replace=“a, t, g or c”
  unsure 1098..1101: /replace=“a, t, g or c”
  unsure 1104: /replace=“a, t, g or c”
  unsure 1107..1110: /replace=“a, t, g or c”
  unsure 1119: /replace=“a, t, g or c”
  unsure 1129..1380: /replace=“a, t, g or c”
nnnnnnnnnnnnnnnnnnnnnnnnnnnnnnnnnnnnnnnnnnnnnnnnnnnnnnnnnnnnaarathytrttygcnnnnnnnccnnnngayggncayttyaayccnytracnnnnytrgcnnnnnnnnnnnnnnnnnnnggntgtgaygtnmgrtggtaynnnnnnnnnnnnnnnnnnnnnnnnnnnnnnnnnnnnnnnnnnnnnnnnttyccngarmgrnnnnnnnnnnnnnnnnnnnnnnnnnnnnnnnnnnnnnnnnnnnnnnnnnnnnnnnnnnnnnnnnnnnnnnnnnnnnnnttygaynnnnnnnnnnnnttynnnnnnmgrnnnnnngartaynnnnnngaynnnnnnnnnnnnnnnnnnnnnttyccnttynnnnnnnnnnnnnnngaynnnnnnttynnnnnnnnnnnnttynnnnnnnnnnnnnnnnnnnnnnnnnnnnnnnnnnnnnnnnnnnnnccnytrnnngaragynnnnnnnnnytrccnccnnnnggnnnnggnnnnnnnccnnnnnnnnnnnnnnnnggnaarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnytrcarnnnggnnnnccnggnttygartaynnnmgrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacncarggnacnnnngaraarnnnnnnnnnaarnnnnnnnnnccnacnytrgargcnttymgrnnnnnnnnnnnnytrgtnnnnnnnacnacnggnggnnnnnnnnnnnnnnnnnnnnnnnnnnnnnnnnnnnnnnnnnnnnnnnnnathgargayttnathccnttnnnnnnngtnatgccnnnnnnngaygtntayathacnaayggnggntayggnggngtnatgytrnnnathnnnaaynnnytrccnnnngtnnnngcnggnnnncaygarggnaaraaygarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 3 - protein, 459 aa, Artificial Sequence
  glycosyltransferase GTC
MSNLFSSQTNLASVKPLKGRKILFANFPADGHFNPLTGLAVHLQWLGCDVRWYTSNKYADKLRRLNIPHFPFRKAMDIADLENMFPERDAIKGQVAKLKFDIINAFILRGPEYYVDLQEIHKSFPFDVMVADCAFTGIPFVTDKMDIPVVSVGVFPLTETSKDLPPAGLGITPSFSLPGKFKQSILRSVADLVLFRESNKVMRKMLTEHGIDHLYTNVFDLMVKKSTLLLQSGTPGFEYYRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 4 - DNA, 1380 bp, Artificial Sequence
  glycosyltransferase GTC
atgagtaatttattttcttcacaaacgaaccttgcatctgtaaaacccctgaaaggcaggaaaatactttttgccaacttcccggcagatgggcattttaatccattgacaggactggctgttcacttacaatggctgggttgtgatgtacgctggtacacttccaataaatatgcagacaaactgcgaagattgaatattccgcattttcctttcagaaaagctatggatatagctgacctggagaatatgtttccggagcgtgatgccattaaaggccaggtagccaaactgaagttcgacataatcaatgcttttattcttcgcgggccggaatactatgttgacctgcaggagatacataaaagttttccatttgacgtaatggtcgctgattgcgcttttacaggaattccttttgtaacagataaaatggatatacctgttgtttctgtaggtgtgttccctcttaccgaaacatcgaaagatcttcctcccgccggcctcgggattacgccttccttttctttacccggaaaatttaaacaaagcatactacggtcggtggctgacctggtcttattccgcgagtccaataaagtaatgagaaaaatgctgaccgaacatggcattgatcatctctatacaaatgtatttgacctgatggtaaaaaaatcaacgctgctattgcaaagcggaacaccgggttttgaatattaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 5 - protein, 440 aa, Artificial Sequence
  glycosyltransferase GTD
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGENVRFVGALLPYSESKSRQPWFDQKLLQYGRIVLVTQGTVEHDINKILVPTLEAFKNSETLVIATTGGNGTAELRARFPFENLIIEDFIPFDDVMPRADVYVTNGGYGGTLLSIHNQLPMVAAGVHEGKNEVCSRIGHFGCGINLETETPTPDQIRESVHKILSNDIFKKNVFRISTHLDVDANEKSAGHILDLLEERVVCG

SEQ ID NO: 6 - DNA, 1323 bp, Artificial Sequence
  glycosyltransferase GTD
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagcgacctgggcgaaaatgtccgttttgtcggcgcattgctgccgtactcggaaagtaaatcccggcagccctggtttgatcagaaacttttacaatatggcaggattgtgctggttacccagggcactgttgagcacgatatcaacaagatacttgtacccacgctggaagctttcaaaaattctgagacgctggtaattgccacaacaggcggtaatgggacagcggaattgcgcgcgcgttttcctttcgaaaacctgatcatcgaagatttcattccgtttgacgatgtgatgcccagagcagacgtttatgttaccaatggtggctatggaggcaccttgctcagcatacataatcagttgccaatggtagcggcgggcgtgcatgagggtaaaaatgaagtttgctcacgtatcggccacttcggctgtgggattaatctggaaacggaaacacctaccccagatcagatacgcgaaagtgtccacaaaatcctgtctaatgacatcttcaaaaagaatgtcttcaggatttcgacgcacttggatgtggatgcgaatgaaaaaagcgcgggtcacattcttgacttgttggaagagcgggttgtttgcggttaa

SEQ ID NO: 7 - protein, 441 aa, Artificial Sequence
  glycosyltransferase GTF
MTTKKILFATMPMDGHFNPLTGLAVHLHNQGHDVRWYVGGHYGAKVKKLGLIHYPYHKAQVINQENLDEVFPERQKIKGTVPRLRFDLNNVFLLRAPEFITDVTAIHKSFPFDLLICDTMFSAAPMLRHILNVPVAAVGIVPLSETSKELPPAGLGMEPATGFFGRLKQDFLRFMTTRILFKPCDDLYNEIRQRYNMEPARDFVFDSFIRTADLYLQSGVPGFEYKRSKMSANVRFVGPLLPYSSGIKPNFAHAAKLKQYKKVILATQGTVERDPEKILVPTLEAFKDTDHLVVITTGGSKTAELRARYPQKNVIIEDFIDFNLIMPHADVYVTNSGFGGVMLSIQHGLPMVAAGVHEGKNEIAARIGYFKLGMNLKTETPTPDQIRTSVETVLTDQTYRRNLARLRTEFAQYDPMALSERYINELLAKQPRKQHEAVEAI

SEQ ID NO: 8 - DNA, 1326 bp, Artificial Sequence
  glycosyltransferase GTF
atgacaactaaaaaaatcctgtttgccaccatgccaatggatggccacttcaaccccctgactggtctggctgttcatttgcataaccagggtcacgacgtacgctggtacgtgggcggacactacggtgccaaagtgaaaaagctgggcctgattcattacccttaccataaagcccaggttatcaatcaggagaatctggacgaggttttccctgaacgtcagaagatcaaagggaccgtaccccggctgcgctttgacctcaacaatgtcttcctgctgcgcgctcccgaattcattaccgacgttacggccatccacaaatcattcccattcgatctgctcatatgcgacaccatgttctcagcggctcccatgctgcgccatattctgaacgttccggtagcggccgtaggcattgtgcccctgagtgaaacctcgaaagaactgccaccggccggcctgggtatggagcctgctaccggtttctttgggcggctgaagcaggacttcctgcgctttatgactacccgtatcctcttcaagccctgcgacgatttgtacaacgagatccggcagcgctataacatggaaccagcccgtgattttgtcttcgactcgtttatccgcaccgccgatttgtacctgcaaagtggtgtaccgggctttgaatacaaacggagcaagatgagtgctaacgtccggtttgtcggcccgcttctcccctactccagcggtattaagccaaactttgcccatgcggccaaactgaagcagtataaaaaggtaattctggccacgcagggcacggtagaacgcgatccggagaagattctggtgccgacgctcgaagcgttcaaagacaccgatcacctggtcgtcataacaacgggcggttctaaaacggccgagttgcgcgcccggtatccgcagaaaaatgtcatcatcgaagacttcattgactttaacctcatcatgccccatgccgacgtatacgtaaccaattcgggtttcggcggagtgatgctgagcattcagcatggcctgccaatggtagctgccggtgttcacgagggtaaaaacgagattgcagcccgcattggctatttcaaactggggatgaatctgaagacagaaacccctacgccggaccagatccggacaagcgtcgaaacggttctgaccgatcagacctaccgccggaacttagcccggttgcggacggagttcgctcagtacgacccaatggcgttgagtgagcgatatatcaacgagctgctggccaaacaaccgcgcaagcaacacgaagccgtagaagcaatctaa

SEQ ID NO: 9 - protein, 454 aa, Segetibacter koreensis
  GT sequence
MKYISSIQPGTKILFANFPADGHFNPLTGLAVHLKNIGCDVRWYTSKTYAEKIARLDIPFYGLQRAVDVSAHAEINDVFPERKKYKGQVSKLKFDMINAFILRSTEYYEDILEIYEEFPFQLMIADITFGAIPFVEEKMNIPVISISVVPLPETSKDLAPSGLGITPSYSFFGKIKQSFLRFIADELLFAQPTKVMWGLLAQHGIDAGKANIFDILIQKSTLVLQSGTPGFEYKRSDLSSHVHFIGPLLPYTKKKERESWYNEKLSHYDKVILVTQGTIEKDIEKLIVPTLEAFKNSDCLVIATTGGAYTEELRKRYPEENIIIEDFIPFDDVMPYADVYVSNGGYGGVLLSIQHQLPMVVAGVHEGKNEINARVGYFDLGINLKTERPTVLQLRKSVDAVLQSDSYAKNVKRLGKEFKQYDPNEICEKYVAQLLENQISYKEKANSYQAEVLV

SEQ ID NO: 10 - DNA, 1365 bp, Segetibacter koreensis
  GT sequence
atgaaatatatttcatcgatacaaccgggaacaaaaatattatttgccaatttccctgccgatggtcacttcaatccgctgacaggattggctgttcatttaaaaaatattgggtgcgatgtgcgttggtacacttcaaagacatatgccgaaaaaattgccaggttagatatacctttttatggtttgcaaagagccgtagatgtaagtgcccatgcggaaatcaacgacgtttttcccgaaaggaaaaaatacaaaggccaggtaagcaagttgaaatttgatatgataaacgccttcattctgcgctctacggaatattatgaagacatattggaaatatacgaggaatttccttttcagttaatgattgctgacatcactttcggcgctattccttttgtagaagaaaaaatgaatattccggttatttccatcagcgttgttccgcttcccgaaacctcaaaagatctggctccctccggccttggtatcaccccttcttattcgttttttggcaaaataaaacagagctttttacgctttattgccgacgaattactttttgcgcaacccactaaagtaatgtggggccttttggcccaacatggaattgatgcggggaaagccaacatatttgacatacttatacaaaaatcaacactggtactacaaagcggcactccgggttttgaatacaagagaagtgacttaagcagtcatgtgcattttattggtccgctgctgccttacacaaaaaagaaagaaagagaaagctggtacaatgaaaagttaagccactacgataaagttattcttgtaacacaaggcacaattgaaaaagatattgagaagcttattgtgccaactcttgaagcatttaaaaactccgattgcctcgttattgctactactggcggtgcctatactgaagagttgagaaaacgttaccccgaggaaaatataattatagaagattttatcccttttgatgatgtaatgccttatgcagacgtatatgtttcaaacgggggatatggcggagttcttttatctatacaacatcaactgcctatggtagtggctggtgtacatgaaggaaaaaatgagattaatgcaagagtgggatattttgatttgggcattaatcttaagaccgaaagacctaccgtacttcaattaagaaaaagtgttgacgcagtcttacaaagtgattcatacgcgaagaatgtaaaacggcttggtaaagaattcaaacaatatgatccgaatgaaatatgtgaaaaatatgtagcgcaactgctggaaaatcaaatttcttataaagaaaaagcaaatagctaccaggccgaagttttggtttaa

SEQ ID NO: 11 - protein, 447 aa, Flavihumibacter solisilvae
  GT sequence
MNHKHSRKILMANVPADGHFNPLTGIAVHLKQQGYDVRWYGSDVYSKKAAKLGIPYFPFSKALEVNSENAEEVFPERKRINSKIGKLNFDLQNFFVRRAPEYYADLIDIHREFPFDLLIADCMFTAIPFVKELMQIPVLSIGIAPLLESSRDLAPYGLGLHPARSWAGKFRQAGLRWVADNILFRKSINVMYDLFEEYNIPHNGENFFDMGVRKASLFLQSGTPGFEYNRSDLSEHIRFIGALLPYAGERKEEPWFDSRLNKFDRVILVTQGTVERDVTKIIVPVLKAFRDSNYLVVATTGGNGTKLLREQYKADNIIIEDFIPFTDIMPYTDVYVTNGGYGGVMLGIENQLPLVVAGVHEGKNEINARIGYFRLGIDLRNERPTPEQMRNAIEKVIANGEYRRNVQALAREFKTYAPLELTERFVTELLLSRRHKLVPVNDDALIY

SEQ ID NO: 12 - DNA, 1344 bp, Flavihumibacter solisilvae
  GT sequence
atgaatcacaaacattccaggaagatcctgatggccaacgtgcctgcggatggccactttaatccgctgaccggcatcgcggttcacctgaagcagcagggctacgatgtacgctggtatggctcggatgtttacagcaaaaaagccgcaaaactgggtattccttattttcctttcagcaaggctcttgaagtaaacagcgaaaatgccgaagaggtctttccggaaagaaaacgcattaacagcaagattggcaagctgaattttgatctgcagaacttctttgttcgccgcgcaccggaatattatgctgacctgatcgacattcaccgcgagttcccttttgacctgctgatcgctgactgtatgtttactgccataccgtttgttaaggaactcatgcagattcctgtgctgtcgatcggaattgcgccactgctggaatcttcccgcgacctggcaccgtatggcctgggccttcatcctgcccgcagctgggccggcaagtttcgccaggcaggcttacgctgggttgcagacaatatccttttccgcaaatccatcaacgtcatgtatgacctttttgaagagtataatatcccgcacaacggggagaatttctttgacatgggtgtaagaaaagcttccctgttcctccagagcggaacaccgggatttgaatataaccgcagcgacctgagtgaacatatccgtttcatcggcgcattgcttccttacgccggagaaagaaaagaagagccctggttcgacagccgcctgaacaaatttgaccgggtgatcctggttacccagggaactgtggaacgtgatgtgacaaagatcattgtgccggtactgaaagccttccgtgacagtaactacctcgtggtagccactaccggcggcaatggaaccaaattgctgcgggagcaatacaaggcagataatatcatcatcgaggattttattcctttcactgatatcatgccctatacggatgtatacgttaccaatggtggttatggtggtgtaatgctggggatagaaaaccagcttccacttgttgttgcaggcgttcacgaagggaaaaatgagatcaatgcaagaataggctatttcaggcttggtatagacctgcgcaacgaaagaccgacaccggaacagatgcgcaatgccattgaaaaagtcattgcaaacggtgaatatcgcaggaatgtgcaggcactggcccgcgaattcaaaacctacgcaccgcttgaattaacggaaaggtttgtgacagaactgctgctcagcaggcgacataaactggttccggtaaacgacgatgcgcttatttactaa

SEQ ID NO: 13 - protein, 463 aa, Cesiribacter andamanensis
  GT sequence
METSQKGGTQSPKPFRRILFANCPADGHFNPLIPLAEFLKQQGHDVRWYSSRLYADKISRMGIPHYPFKKALEFDTHDWEGSFPERSKHKSQVGKLRFDLEHVFIRRGPEYFEDIRDLHQEFPFDVLVAEISFTGIAFIRHLMHKPVIAVGIFPNIASSRDLPPYGLGMRPASGFLGRKKQDLLRFLTDKLVFGKQNELNRQILRSWGIEAPGHLNLFDLQTQHASVVLQNGTPGFEYTRSDLSPNLVFAGPLLPLVKKVREDLPLQEKLRKYKNVILVTQGTAEQNTEKILAPTLEAFKDSTWLVVATTGGAGTEALRARYPQENFLIEDYIPFDQIMPNADVYVSNGGFGGVLQAISHQLPMVVAGVHEGKNEICARVGYFKLGLDLKTETPKPAQIRAAVEQVLQDPQYRHKVQALSAEFRQYNPQQLCEHWVQRLTGGRRAAAPAPQSAGGQLLSLTLN

SEQ ID NO: 14 - DNA, 1392 bp, Cesiribacter andamanensis
  GT sequence
atggaaacttcacaaaaaggcgggactcagtcacccaaaccattcagaagaattctttttgccaactgcccggccgacgggcactttaatccgctcattccactggcggaattcctcaagcagcaggggcatgatgtgcgctggtactcctcccgcctgtatgccgataagatttcgcgcatgggcattccccattatccttttaaaaaggcgcttgaatttgacacccacgactgggaagggagctttcccgagcgcagcaaacacaaaagccaggtaggcaagctgcgcttcgatctggagcatgtgttcattcgccgcggccctgagtactttgaagatattcgagacctccaccaggagtttccctttgatgtgctggtggccgagatcagctttaccggtattgcattcatccgccacctgatgcacaagccggtgattgcggtgggcatttttcccaacatcgcatcttcgcgcgacttgcctccctatgggctgggcatgcgtcctgctagcgggtttctgggtagaaaaaagcaagacctgctgcgctttcttaccgacaagctggtgtttggaaaacagaacgagctgaatcggcagattctccgcagctggggaattgaggcccccgggcaccttaacctgtttgacctgcagacgcagcatgcctcggtggttttgcagaacggaaccccgggttttgagtacacccgcagcgacctgagtcccaacctggtatttgcaggccccctgttgccgttggtgaaaaaagtgcgggaagatctacccctgcaggagaagctcaggaagtacaaaaacgtaattctggtaacccagggcactgccgagcaaaataccgaaaagattctggcgcccacactggaagcctttaaagacagcacctggctggtggtggcaaccacaggaggagcgggcaccgaggcgctgagggccaggtatccccaggagaatttcctgatcgaagattatattccttttgatcagatcatgcccaatgccgatgtatatgtatcgaacggaggctttggaggcgtcctgcaggccatttcacaccaactgcccatggtagtggcaggggtacatgagggtaaaaatgagatctgtgcccgggtgggctattttaagctggggctcgacctgaagacggaaacccccaaaccagcccagataagagcggcggtagagcaggtgctgcaagacccccagtaccgccacaaggtgcaggccctgagtgctgaattccggcaatacaatccacaacagctgtgcgagcactgggtgcagcgcctgacaggcggacgtagagcggctgcacccgcacctcagtcggctggcgggcagctactttccctgacgctgaactaa

SEQ ID NO: 15 - protein, 450 aa, Niabella aurantiaca
  GT sequence
MYTKTANTTNAAAPLHGGEKKKILFANIPADGHFNPLTGLAVRLKKAGHDVRWYTGASYAPRIEQLGIPFYLFNKAKEVTVHNIDEVFPERKTIRNHVKKVIFDICTYFIERGTEFYEDIKDINKSFDFDVLICDSAFTGMSFVKEKLNKHAVAIGILPLCASSKQLPPPIMGLTPAKTLAGKAVHSFLRFLTNKVLFKKPHALINEQYRRAGMLTNGKNLFDLQIDKATLFLQSCTPGFEYQRAHMSRHIHFIGPLLPSHSDAPAPFHFEDKLHQYAKVLLVTQGTFEGDVRKLIVPAIEAFKNSRHLVVVTTAGWHTHKLRQRYKAFANVVIEDFIPFSQIMPFADVFISNGGYGGVMQSISNKLPMVVAGIHEGKNEICARVGYFKTGINMRTEHPKPEKIKTAVNEILSNPLYRKSVERLSKEFSEYDPLALCEKFVNALPVLQKP

SEQ ID NO: 16 - DNA, 1353 bp, Niabella aurantiaca
  GT sequence
atgtacacaaaaacagcaaacacaaccaatgccgctgctcccttacacggcggtgaaaaaaagaaaatcttatttgccaacatccctgccgacgggcatttcaaccctctaacgggattagccgttcggctcaaaaaagcagggcatgatgtccgctggtacaccggcgccagctatgcaccccgtatcgaacagctgggcattcccttctatctttttaacaaggcaaaagaggtaaccgttcacaacattgacgaagtatttcccgaaaggaaaacgatccggaatcatgtaaagaaagtcatctttgatatctgcacgtattttatcgaacgcggaacagaattttatgaagacataaaggacatcaataaaagtttcgatttcgacgtgctgatctgcgacagcgcttttaccggtatgtcgttcgtaaaagaaaaactaaacaagcatgcagtagccatcggcatcctccctttatgtgcctcttcgaaacagctacccccgcccatcatgggacttacaccggccaaaaccctggcaggaaaagccgtgcattcgtttttgcgttttcttaccaataaagtattgtttaaaaagccccacgcgctgatcaacgaacaataccgccgtgcaggcatgctgaccaatggcaaaaacctgtttgatctgcagatcgataaggcaacactgtttttacaaagctgtaccccggggtttgaataccaacgcgcgcatatgagccggcatatccattttataggccctttactgccctcccatagtgatgcccctgccccattccattttgaagacaaactgcatcagtatgcaaaagtgctgctggtaacgcagggaacctttgaaggagatgtgcgcaagctgatcgtgcccgcaattgaagcctttaaaaacagccgccacctggtggtggtaacaacggccggatggcatacccataaactgcgccagcggtataaagcatttgccaatgttgttattgaagactttattccgttcagccagatcatgccttttgccgatgtattcatttcaaacggtggttacggcggtgtgatgcaaagcataagcaataagctgccaatggtagtggccggcatacacgaagggaaaaacgaaatatgtgcccgggtgggatattttaaaacaggcatcaatatgcgcacggaacatcccaaaccggaaaaaataaaaacagctgtgaacgagatcctgagcaaccccctttaccggaaaagcgtggaacggctttcgaaggaattttcggagtacgacccgttggccctttgtgaaaaattcgtcaacgctttacccgtccttcagaaaccatag

SEQ ID NO: 17 - protein, 441 aa, Spirosoma radiotolerans
  GT sequence
MITPQRILFATMPMDGHFSPLTGLAVHLSNLGHDVRWYVGGEYGEKVRKLKLHHYPFVNARTINQENLEREFPERAALKGSIARLRFDIKQVFLLRAPEFVEDMKDIYQTWPFTLVVHDVAFIGGSFIKQLLPVKTVAVGVVPLTESDDYLPPSGLGRQPMRGIAGRWIQHLMRYMVQQVMFKPINVLHNQLRQVYGLPPEPDSVFDSIVRSADVYLQSGVPSFEYPRKRISANVQFVGPLLPYAKGQKHPFIQAKKALQYKKVILVTQGTIERDVQKIIVPTLEAFKNEPTTLVIVTTGGSQTSELRARFPQENFIIDDFIDFNAVMPYASVYVTNGGYGGVMLALQHNLPIVVAGIHEGKNEIAARIDYCKVGIDLKTETPSPTRIRHAVETVLTNDMYRQNVRQMGQEFSQYQPTELAEQYINALLIQEKSSRLAVVA

SEQ ID NO: 18 - DNA, 1326 bp, Spirosoma radiotolerans
  GT sequence
atgatcacaccccaacgcattttgtttgctaccatgccaatggatggccattttagtcctctcaccggtcttgccgttcacttaagtaaccttggccacgatgtccgctggtatgtgggcggtgagtacggcgaaaaagtacggaagcttaagttgcaccattatccattcgtgaacgcccgaaccatcaatcaggaaaatctggagcgtgagtttccggaacgggccgcccttaagggttcgattgcccggctacggttcgatattaagcaggtgtttctgcttcgtgctccggaattcgttgaggatatgaaagatatctaccagacgtggccgttcactctggtagtacatgatgtagccttcattgggggctcgttcattaagcaactattgcccgttaaaaccgtggcggtaggcgtagtacccctcacggagtcggacgattacctgccgccgtctggtctgggcaggcaacccatgcgcggcatagctggccgctggattcagcatctgatgcgctacatggtgcagcaggttatgttcaaacccatcaatgtcctgcacaatcaacttcgacaggtctatggtctgccgcctgagccggactccgtgttcgattcgatcgtacgttctgccgatgtttatctccaaagtggcgtacccagctttgagtaccctcgcaaacggataagtgccaatgttcagtttgtggggccgctgctcccctacgccaaaggtcaaaagcacccgtttatacaggcaaaaaaagcgttgcagtacaaaaaagttattttagtaactcaggggacgatagagcgggatgtccaaaaaatcattgtaccaaccctggaagcttttaaaaatgagcctactacgctggtgatcgtcacaactggtggctcccaaacgagtgagttgcgtgcgcgttttccgcaggaaaatttcattattgatgactttatcgattttaatgcggttatgccctatgccagtgtgtatgtaacaaacgggggctatggcggggtaatgcttgcgctgcaacacaacctgccgattgtcgtcgcgggaattcacgagggtaaaaacgagattgcagcccgcattgattactgtaaggtaggcatagacctgaagactgagacgcccagccccacccgcattcgccatgccgtcgaaactgtattgaccaatgacatgtaccggcagaatgtccgtcaaatggggcaagagttcagtcagtatcaaccaactgaactggcggaacaatacatcaatgcgcttttaatacaagagaaaagctcccggctggccgttgtggcctag

SEQ ID NO: 19 - protein, 440 aa, Fibrella aestuarina
  GT sequence
MNPQRILFATMPFDGHFSPLTNLAVHLSQLGHDVRWFVGGHYGQKVTQLGLHHYPYVKTRTVNQENLDQLFPERATIKGAIARIRFDLGQIFLLRVPEQIDDLRAIYDEWPFDLIVQDLGFVGGTFLRELLPVKVVGVGVVPLTESDDWVPPTSLGMKPQSGRVGRLVSRLLNYLVQDVMLKPANDLHNELRAQYGLRPVPGFIFDATVRQADLYLQSGVPGFEFPRKRISPNVRFIGPMLPYSRANRQPFEQAIKTLAYKRVVLVTQGTVERNVEKIIVPTLEAYKKDPDTLVIVTTGGSGTLALRKRYPQANFIIEDFIDFNAVMPYVSVYVTNGGYGGVMLALQHKLPIVAAGVHEGKNEIAARIGYCQVGVDLRTETPTPDQIRRAVATILGDETYRRQVRRLSDEFGRYNPNQLAEQYINELLAQSVGEPVAALS

SEQ ID NO: 20 - DNA, 1323 bp, Fibrella aestuarina
  GT sequence
atgaatccccaacgcatcctcttcgccaccatgccattcgacgggcactttagccccctcaccaacctggccgttcaccttagccaactcgggcacgatgtgcgctggtttgtgggtgggcattacggccagaaagtaacgcagctgggcctgcaccattacccgtacgtgaaaacgcgcaccgtcaatcaggaaaatctggatcagctcttccccgaacgggccaccatcaaaggcgccattgcccgcatccgtttcgacctgggccagattttcctgcttcgtgtgcccgaacagatcgacgacctcagggcgatttacgacgaatggccgtttgacctcattgtgcaggatctgggctttgtggggggtacgttcctgcgcgagctgctgccggtgaaggtagtgggcgtgggcgtggtgccactcaccgaatccgacgactgggtgcccccgaccagcctgggcatgaaaccgcagtcgggccgggtgggccggctggtaagtcggctgctcaactacctggtgcaggacgttatgctgaagcccgccaatgacctgcacaacgagttaagggcgcagtacggccttcggccggtgccgggttttatctttgatgccaccgttcggcaggccgatctgtacctgcaaagcggcgtgccgggttttgaatttccccgtaagcgcatcagccccaacgtgcggttcatcgggcccatgctgccctacagccgggcaaacaggcagccgtttgagcaggccatcaaaacgctggcctataagcgggtggtgctcgtcacgcaggggaccgtcgagcggaacgtggagaagatcatcgtgcccacgctggaagcctacaaaaaagatcccgatacgctggtgattgtgaccaccggcggctcaggtacgttggcgttgcggaaacggtacccacaggccaattttatcatcgaagactttatcgatttcaacgccgtgatgccctacgtgagtgtgtacgtgaccaacggcgggtatggcggcgtgatgctggcgctgcaacacaagctcccgattgtggcggcgggcgtgcatgaaggcaaaaacgaaatcgccgcccggatcggctactgccaggtgggtgtcgacctgcgcaccgaaacgcccacccccgaccagattcgccgggcggtggccaccatcctgggcgacgaaacctaccggcgtcaggtacgtcggttgagcgacgagtttggccggtataaccctaatcaactggccgaacagtacatcaacgagctactggcccagtcggtgggggagcccgttgccgccctgtcgtga

SEQ ID NO: 21 - protein, 434 aa, Aquimarina macrocephali
  GT sequence
MTRMSQKKILFACIPADGHFNPMTAIAIHLKTKGYDVRWYTGEGYKNTLHRIGIPYLPFQNAQELKIEEIDKMYPDRKMLKGIAHIKFDIINLFINRMKGYYEDIAEIHQVFPFDILVCDNTFPGSIVKKKLNIPIASIGVVPLALSAPDLPLYGIGHQPATTFFGKRKQNFIKLMADKLIFDETKVVYNQLLRSLDLSEEENLTIFDIAPLQSDVFLQNGIPEIDYPRYSLPESIKYVGALQVQTNNNNNQKLKKDWSAILDTSKKIILVSQGTVEKNLDKLIIPSLEAFKDSDYIVLVATGYTDTKGLQKRYPQQHFYIEDFIAYDAVMPHIDVFIMNGGYGSALLSIKHGVPMITAGVNEGKNEICSRMDYSGVGIDLKTEKPRAVTIQNATERILGTDKYLDTIQKIQQRMNSYNTLDICEQHISRLISE

SEQ ID NO: 22 - DNA, 1305 bp, Aquimarina macrocephali
  GT sequence
atgacacgaatgtcccaaaaaaaaattcttttcgcttgtatacctgcagacggtcattttaatcctatgacagctatagctattcatctaaaaacaaaagggtatgatgtaagatggtatactggggagggctataaaaacacactacacagaatagggataccttatttaccgttccaaaatgcgcaggagcttaaaattgaggagatagataaaatgtatccagatcgaaaaatgctaaaaggaatcgcacatattaagttcgatattattaatctgtttattaatagaatgaaagggtactatgaagatatcgcagagatacatcaagtttttccgtttgatattttggtatgtgacaacacttttcccgggtctattgttaagaaaaaacttaatatcccaattgctagtataggagttgtgcctttagcactttctgcacctgatcttccattatacggcattggtcatcagcctgctacaacttttttcggtaagagaaaacagaactttataaaactaatggcagataaactcatttttgatgaaacaaaagtagtatataatcaattattacgctcattggatttatccgaagaagaaaatctaactatttttgatatagctccattacaatcggatgtttttttgcaaaacggaattcctgagatcgattatccaaggtatagtcttcccgaatccataaaatacgttggagcactacaagtacagaccaacaataacaacaatcaaaagttaaaaaaggactggagtgctattttagatacgtcaaaaaaaatcatattagtatctcagggaaccgtagaaaaaaatcttgacaagcttattattccttctttagaagcttttaaagactcagattacatagtactggtagctactggttataccgacactaaaggtttacaaaaacgataccctcagcagcatttttatatcgaagatttcatagcctatgatgctgtaatgccacatatagatgtctttatcatgaatggaggatatggcagtgctttactaagtattaaacacggtgtaccaatgattaccgctggggttaacgaaggtaaaaatgaaatctgttcccgaatggattattctggagtcggtattgatctaaaaacagaaaaaccacgagcagtcacaatacaaaatgcaactgaaagaatattaggtacagataaatatttagacactatacagaaaatacaacagcgtatgaattcttataacacattagatatctgcgaacaacatatctcccgtcttatttcagaataa

SEQ ID NO: 23 - protein, 452 aa, Artificial Sequence
  Chimera 1
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 24 - DNA, 1359 bp, Artificial Sequence
  Chimera 1
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 25 - protein, 447 aa, Artificial Sequence
  Chimera 2
MSNLFSSQTNLASVKPLKGRKILFANFPADGHFNPLTGLAVHLQWLGCDVRWYTSNKYADKLRRLNIPHFPFRKAMDIADLENMFPERDAIKGQVAKLKFDIINAFILRGPEYYVDLQEIHKSFPFDVMVADCAFTGIPFVTDKMDIPVVSVGVFPLTETSKDLPPAGLGITPSFSLPGKFKQSILRSVADLVLFRESNKVMRKMLTEHGIDHLYTNVFDLMVKKSTLLLQSGTPGFEYYRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFDDVMPRADVYVTNGGYGGTLLSIHNQLPMVAAGVHEGKNEVCSRIGHFGCGINLETETPTPDQIRESVHKILSNDIFKKNVFRISTHLDVDANEKSAGHILDLLEERVVCG

SEQ ID NO: 26 - DNA, 1344 bp, Artificial Sequence
  Chimera 2
atgagtaatttattttcttcacaaacgaaccttgcatctgtaaaacccctgaaaggcaggaaaatactttttgccaacttcccggcagatgggcattttaatccattgacaggactggctgttcacttacaatggctgggttgtgatgtacgctggtacacttccaataaatatgcagacaaactgcgaagattgaatattccgcattttcctttcagaaaagctatggatatagctgacctggagaatatgtttccggagcgtgatgccattaaaggccaggtagccaaactgaagttcgacataatcaatgcttttattcttcgcgggccggaatactatgttgacctgcaggagatacataaaagttttccatttgacgtaatggtcgctgattgcgcttttacaggaattccttttgtaacagataaaatggatatacctgttgtttctgtaggtgtgttccctcttaccgaaacatcgaaagatcttcctcccgccggcctcgggattacgccttccttttctttacccggaaaatttaaacaaagcatactacggtcggtggctgacctggtcttattccgcgagtccaataaagtaatgagaaaaatgctgaccgaacatggcattgatcatctctatacaaatgtatttgacctgatggtaaaaaaatcaacgctgctattgcaaagcggaacaccgggttttgaatattaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagatttcattccgtttgacgatgtgatgcccagagcagacgtttatgttaccaatggtggctatggaggcaccttgctcagcatacataatcagttgccaatggtagcggcgggcgtgcatgagggtaaaaatgaagtttgctcacgtatcggccacttcggctgtgggattaatctggaaacggaaacacctaccccagatcagatacgcgaaagtgtccacaaaatcctgtctaatgacatcttcaaaaagaatgtcttcaggatttcgacgcacttggatgtggatgcgaatgaaaaaagcgcgggtcacattcttgacttgttggaagagcgggttgtttgcggttaa

SEQ ID NO: 27 - DNA, 1380 bp, Artificial Sequence
  codon optimized GTC sequence
atgtcaaacctgttctcatctcaaacaaacctggcctcggtaaaaccgttaaaaggtcgtaaaatccttttcgcaaattttcccgctgatggacactttaatccgttaactgggttagcagtccatttacaatggcttggttgcgatgtgcgttggtacacttcaaataagtacgccgataagcttcgtcgccttaacatccctcacttcccttttcgtaaggccatggatattgctgacttagaaaacatgtttcctgagcgtgatgccatcaaaggacaggtcgcaaaactgaagttcgacattattaatgctttcattctgcgcggccctgagtactacgtcgacttacaagaaattcataaatcctttccctttgacgttatggtcgctgattgcgcgtttacgggaatcccgttcgtaactgacaaaatggatattcccgtcgtatcggtcggggtctttccactgaccgagacttctaaagatttgcctccggccggattgggtattactccctcgttttccttgccaggtaagttcaagcaatcgattttacgcagtgtggccgatttggtgttatttcgtgagagcaataaggtcatgcgcaaaatgttgactgagcatggtattgaccacctttacacaaacgtatttgatcttatggttaaaaaatcaacgttactgttgcagtcagggactccgggcttcgagtattaccgtagtgatcttggtaagaatattcgttttatcggaagcttgcttccctatcagagcaaaaaacagactactgcttggagtgatgagcgtctgaatcgctatgaaaaaatcgtcgtagtcactcagggaactgtagagaaaaacatcgaaaagattttggtgccaacccttgaggctttccgcgacactgacctgcttgtgatcgcgacgacgggaggttcaggaaccgctgaattgaaaaaacgttaccctcagggcaacttaatcattgaggacttcattccatttggtgacattatgccatacgctgatgtatatatcaccaatggtggttacggcggagttatgcttggcatcgaaaatcaactgccccttgtcgtagccgggatccacgaaggaaagaacgagatcaacgcacgtattgggtactttgagcttggaatcaatctgaaaacggagtggccgaagccagagcagatgaaaaaagcgattgacgaagttatcggtaataagaagtacaaagagaatatcacaaaactggcgaaggaattctcaaactaccatcctaacgaattgtgcgcccaatacatctctgaagtcttacagaagaccggccgcttgtacatttcgtccaagaaggaagaagaaaagatttactaa

SEQ ID NO: 28 - DNA, 1323 bp, Artificial Sequence
  Codon optimized GTD sequence
atgaccaaatacaaaaatgagttgaccggcaaacgtattttgtttggaaccgtgcctggagatggacatttcaaccccttaacaggcttagccaagtacctgcaagaactgggctgcgatgtacgctggtatgcatctgatgtatttaagtgcaaactggagaagctgagcatccctcactatgggttcaagaaggcttgggatgtaaatggagtaaatgttaatgaaattcttccggagcgtcaaaagctgaccgaccctgcggaaaagctgagtttcgaccttatccacatttttggaaatcgcgctcctgaatattacgaggacatcttggaaattcacgagagttttcctttcgacgtcttcatcgccgactcctgcttcagtgctattcccttagtttccaagcttatgtctattcctgtcgtggcagtaggggtgatcccgctggcagaagagagtgtggacttagcaccatacggaactggcctgccgccagctgcgacagaagagcagcgcgccatgtatttcggcatgaaggacgcacttgccaacgtggtgttcaaaacagccattgactcgttttccgccattttagatcgttatcaagtgcctcacgagaaagcgatcttatttgatactcttattcgtcaaagcgatttgtttttgcaaatcggagccaaagctttcgagtatgaccgcagcgatttgggggaaaacgtgcgtttcgttggagccctgctgccttattcggagagcaaaagtcgtcaaccctggttcgatcaaaagttgttacaatatgggcgcattgtcttggtcactcaggggacggtggaacatgatattaataagattctggttcctactttagaggcatttaaaaactcggaaaccctggtcatcgcgacaacaggaggaaatggtacagcagaattacgtgcgcgctttcccttcgaaaacttgatcattgaggatttcattccgttcgacgacgtgatgccccgcgcggatgtatatgtcaccaatggaggctatggtggcacgctgctttcaattcacaaccaacttccgatggttgcagccggggtccatgagggcaaaaatgaggtgtgttcccgtatcgggcactttggctgtgggatcaatctggagacggagacgccgacaccagatcagattcgtgaatcagttcataaaatcctgtcgaacgacattttcaagaaaaacgtttttcgtatttcaactcatttggacgtcgatgctaacgagaaaagcgccggtcatatcttggatctgttggaggagcgtgtcgtttgtgggtaa

SEQ ID NO: 29 - DNA, 1326 bp, Artificial Sequence
  Codon optimized GTF sequence
atgacgaccaagaagattcttttcgcaactatgcctatggacggtcatttcaatcctctgacagggcttgcggtgcacttgcataaccaaggtcatgatgtccgctggtacgtcggcggacattatggcgcaaaggttaaaaaattaggattaattcattatccctatcacaaggctcaagtcattaatcaagaaaatctggacgaagtcttcccggagcgtcaaaagatcaaaggcactgtaccacgtttacgtttcgatcttaataatgtgttcttgctgcgcgctcccgaatttattaccgatgtcactgcgattcacaaatcgtttccttttgacctgctgatctgtgataccatgttctcggcggctccaatgttacgccacattttgaatgtacccgtcgcagcggtgggtattgtgccattgtcagaaacttccaaggaactgccgccagcggggttggggatggagccggcgacaggattctttggacgtttgaagcaggatttcttacgtttcatgaccactcgtatcctttttaagccgtgcgacgatttatacaacgagatccgccagcgctacaacatggagcccgcccgcgattttgtctttgactccttcatccgtacggcggatctgtacctgcagtcaggcgtccctggatttgagtacaagcgctcaaagatgtcggcgaatgtgcgtttcgtcggacccttactgccctatagttcagggatcaagcctaattttgcccatgccgctaaattgaaacagtacaaaaaggtcatcttagccacccagggaacagtcgagcgtgaccctgagaaaatcttagtaccaactcttgaagctttcaaggacaccgatcatctggttgtgattacgaccggaggctcgaagacagcggagctgcgcgctcgttaccctcagaagaacgtgattatcgaggatttcattgactttaacttaatcatgcctcatgcagatgtttacgtcaccaactctggttttggtggtgtgatgctttccattcagcatggtttgccaatggtagctgcaggagttcacgaggggaagaacgaaattgctgctcgcattgggtatttcaaattagggatgaacttgaaaaccgaaacgccgacacccgaccagatccgtacaagtgtagagactgttttgacggaccaaacctatcgtcgcaacttagcgcgtttacgcacggaattcgctcaatacgacccaatggcactgtcagaacgctatattaacgagttgcttgcgaagcagccacgcaaacagcatgaggcagtagaagcgatttaa

SEQ ID NO: 30 - DNA, 1365 bp, Segetibacter koreensis
  Codon optimized GT sequence
atgaaatatatcagctccattcagcccggcacaaaaattttattcgcaaactttccggctgacggacacttcaatccattgacgggcttggcagtgcacttgaaaaatattggctgtgatgtccgttggtacaccagtaaaacctatgccgagaagatcgctcgcctggatatcccattttatggactgcagcgtgcagttgatgtatctgcccacgcagagattaatgacgtgtttcctgaacgcaagaagtacaagggacaagtttcaaaattgaagtttgatatgatcaatgcgtttattctgcgcagtacagagtattacgaggacattttagaaatttatgaggagtttcctttccagcttatgatcgccgacattaccttcggcgcgattcccttcgttgaagaaaaaatgaacattccagtaatctccatttcggttgtaccgttacctgaaacgtcgaaagatcttgccccgagtggcctgggaattacgccatcatactcgttctttggtaaaattaagcaatcgttcttacgtttcattgccgacgagcttttattcgcgcaacctaccaaggtcatgtggggtttattagcccaacacggaattgacgcggggaaagctaacatctttgacatcttgatccaaaagagtacgctggtattgcagagtggtacgccagggtttgagtacaaacgttctgatttgagctcccacgtgcatttcatcggcccgctgttaccctacactaagaagaaggaacgcgaatcatggtataatgaaaaattgtctcattatgataaggtcattttggtaacccaggggacaatcgaaaaagatattgagaaattaattgtaccgactttggaggcctttaagaattccgattgcctggtgattgcgacgacgggtggggcttacactgaagaattgcgcaaacgctatcctgaggaaaatattatcatcgaagacttcattccgtttgacgacgtaatgccgtatgccgacgtttacgtttcgaacggaggctatggtggcgtattgttatcaattcaacaccaactgcctatggtcgtcgcaggagttcatgagggtaaaaacgagatcaatgcgcgtgtaggttactttgaccttggcatcaatttgaagaccgagcgccccaccgttcttcaattgcgcaagagtgtagatgccgtgttacagtccgacagttatgcgaaaaacgtgaagcgtctgggaaaggagtttaagcaatacgatcctaatgaaatctgcgaaaagtacgtagcgcaactgcttgagaatcaaatcagctacaaggagaaggcgaattcctatcaggccgaagttctggtttaa

SEQ ID NO: 31 - DNA, 1344 bp, Flavihumibacter solisilvae
  Codon optimized GT sequence
atgaatcataagcattcgcgtaagatcctgatggcgaacgttcccgccgatggtcattttaatcccctgactggaattgcggtccaccttaagcagcaaggctacgatgtacgttggtatggatcagacgtttatagcaaaaaggcggccaaattagggattccgtatttcccattttcaaaagcgttggaagttaattcagaaaatgcagaagaagtgttccccgaacgtaagcgcatcaattcgaagattggaaaattaaatttcgatttgcagaatttctttgttcgtcgtgcgccagagtactatgccgatcttattgatattcaccgtgagtttcctttcgacttgttaattgcggattgtatgttcacagcgatcccttttgttaaggagttgatgcagatcccggtgctgtctattggcattgcgccattgcttgaatcatcgcgtgatttggctccgtacggattgggtctgcatccggctcgtagctgggcggggaaattccgtcaagcgggactgcgctgggttgctgataacatcctttttcgtaaatcaatcaatgttatgtacgacctgttcgaggaatataatattcctcacaatggagaaaactttttcgacatgggcgttcgtaaagcttcactgttcctgcaatcgggtacgccgggttttgagtacaatcgcagcgatttatctgagcatatccgcttcatcggagcacttcttccgtacgctggtgaacgcaaggaggaaccctggttcgacagtcgcctgaacaaattcgaccgtgtcattctggttacacaagggactgttgaacgtgacgttacaaaaatcatcgtaccagtgttgaaagccttccgtgattcgaattacttggttgtcgcgacgactgggggaaatggtacaaagcttcttcgtgagcagtataaggctgacaatatcattatcgaggacttcattccatttaccgatattatgccctatactgatgtttacgtaactaacgggggctacggtggagtgatgttaggaatcgaaaatcaattacctttagtagtggcaggtgtgcacgaggggaagaatgagatcaatgcccgcatcgggtatttccgcttaggcattgatctgcgtaatgaacgtcccacccccgaacaaatgcgtaacgcgattgaaaaagtaatcgcaaacggagaatatcgtcgcaacgtccaagcgcttgcacgtgagtttaaaacatacgctcccttggagttgaccgagcgtttcgtcacagaactgttgttgtcacgtcgccacaaattggtccccgtcaacgatgacgctttgatctactaa

SEQ ID NO: 32 - DNA, 1392 bp, Cesiribacter andamanensis
  Codon optimized GT sequence
atggagacgagtcaaaaaggaggaacgcagtcgccaaagcccttccgccgtatcttatttgcaaattgtcctgcggatgggcatttcaaccctttaattcctttggctgagtttttgaagcaacaaggtcatgacgtacgctggtatagctcgcgtttatatgcggataagatttcacgtatgggcatcccgcactacccattcaaaaaggcgctggaatttgacacccacgattgggaaggcagctttccagaacgtagcaagcataagtcgcaagtaggcaagttacgttttgatctggaacatgtcttcatccgtcgcgggcccgaatactttgaggatattcgcgatttacaccaggagtttcctttcgatgttttagtggcagaaatcagctttacggggatcgcatttatccgccatctgatgcacaagcccgtgatcgcagtcggcattttcccgaacattgcttcctcacgcgacttacctccatacggactgggcatgcgtccagcttctggatttttgggtcgtaagaaacaggacttactgcgttttttaaccgacaagttggtcttcggtaagcaaaatgagttaaaccgtcaaattcttcgctcatggggcatcgaggctcctggccacctgaatctttttgacctgcagacacagcacgcgtctgtagttcttcagaatggtacccctggatttgagtacacccgttccgatctgagcccaaacttggtatttgctgggcctctgctgcctcttgtcaaaaaggtgcgcgaagatttgccgttgcaggagaaattgcgcaaatataaaaacgtcatcctggtgacacaggggaccgctgaacagaacacagaaaagatcttagctcccacccttgaagcattcaaagactccacttggcttgtcgtggcgacaactggcggagcggggaccgaagctttacgcgctcgctatccacaagaaaatttcttaatcgaggactatattcccttcgatcagatcatgccaaacgcggatgtttatgtgtcgaatggggggttcgggggtgtgcttcaggcgatctcacatcagcttccgatggtggtggccggcgtacacgagggtaaaaatgagatttgcgcccgcgtgggttacttcaaattgggacttgatctgaagaccgagaccccgaagcctgctcaaattcgcgcagcggtagaacaagttcttcaagatccacagtaccgccataaggttcaggcgttgtcagccgaattccgccaatataacccgcagcaattatgcgaacattgggtgcaacgtttaacggggggccgccgtgccgccgcccccgccccgcagtccgccgggggccagttattgagtttgacccttaattaa

SEQ ID NO: 33 - DNA, 1353 bp, Niabella aurantiaca
  Codon optimized GT sequence
atgtatacaaaaaccgcgaacacgaccaacgcggcagcgccattacacggaggcgaaaagaaaaagattttgtttgcaaacatcccagcagatgggcacttcaacccgttgacgggactggctgtccgccttaaaaaagcgggccacgatgtgcgctggtatacgggggcgtcgtatgcaccccgcatcgagcaactggggattcctttttatttattcaacaaagccaaagaagttacagttcataatattgatgaagtattcccagaacgtaaaacgatccgcaatcacgtcaaaaaagtcatcttcgatatctgtacttactttatcgaacgtgggaccgaattctatgaagatattaaagatatcaacaagagcttcgacttcgatgttcttatttgcgatagtgcctttacgggaatgtcctttgtaaaagaaaaattaaataagcatgcagtcgcaattggcattttgcccctttgcgcttcgtctaaacagctgccccccccaattatggggttaactccggcgaagaccctggcaggaaaggctgtgcactcgttccttcgctttcttactaacaaggtattgtttaaaaagccgcatgccttaatcaacgagcagtatcgtcgcgcgggaatgctgacgaacggtaagaacttattcgatttgcagattgataaagctacattattcttgcaatcctgcaccccaggcttcgaataccaacgcgctcatatgtctcgccatatccatttcatcggcccattattgccgtcacactcggatgcgcctgcaccatttcactttgaagacaaacttcatcagtacgctaaggtactgttggtgactcaaggcacattcgaaggtgacgttcgcaagcttattgttcctgcaattgaagcgttcaaaaattcgcgccatttggtagtcgtcacaacggcgggctggcacacccataagctgcgtcagcgctataaagccttcgcgaatgttgttattgaagatttcattcccttctcccaaatcatgccatttgcagacgtcttcattagtaacggtggatatggtggtgtaatgcagtccatttcaaataaactgcctatggtggttgctgggattcatgagggtaaaaatgaaatctgcgctcgcgtgggttatttcaagaccggaattaacatgcgtaccgagcatccaaaaccggaaaagattaaaaccgcagtaaatgagattctttctaatccgttgtatcgcaaatcagtggaacgtctgagtaaagagttctccgaatatgaccccttagcgttatgcgaaaagttcgtcaacgctcttcccgtcttacagaagccctag

SEQ ID NO: 34 - DNA, 1326 bp, Spirosoma radiotolerans
  Codon optimized GT sequence
atgatcactccacagcgcattttgtttgcgacgatgccgatggacggtcatttttctcccctgacgggtcttgccgtgcacttatcgaatttagggcacgatgttcgctggtacgtgggcggagagtatggcgaaaaggtgcgcaagttgaagttgcaccattatccctttgtcaacgctcgcacaattaatcaagagaatcttgagcgtgaattccctgagcgcgccgcgttaaagggtagcattgcccgtcttcgttttgacatcaagcaggtttttctgttgcgtgcaccagaattcgtggaagatatgaaagatatttaccaaacctggccctttacacttgtggttcacgacgtcgcctttattggtggaagctttattaaacagttgttacccgtaaaaacagtagcggttggagtcgtgccacttactgaatcggatgattacttaccaccctccggtcttggccgccaaccgatgcgcggaatcgccggtcgctggatccaacatctgatgcgctacatggttcagcaagtcatgtttaagccaatcaacgtcctgcataaccaacttcgtcaggtctatggtctgcctccggaaccggacagtgtctttgacagtatcgtgcgctctgccgatgtgtacttgcagtccggcgtaccgtcttttgagtatccacgcaagcgtatctcagctaatgttcaatttgtgggccctctgcttccgtatgctaaaggacaaaaacacccctttattcaggccaaaaaagccttgcagtacaagaaggttattctggtaactcaaggtactattgaacgcgatgtgcaaaaaattatcgtcccgacgctggaggcatttaagaacgaaccaacaactttggtcatcgtaacaaccgggggttcccagactagcgagctgcgtgcgcgctttccacaagagaatttcattatcgacgacttcattgattttaatgcagtaatgccatacgcgagcgtttacgtcactaatgggggctatggtggtgttatgttagctctgcaacacaacttgccgattgttgtagcgggaatccatgaaggaaagaacgagattgctgcccgcattgattactgcaaggtcggtatcgacctgaagactgagacccctagtccgacacgcattcgtcacgcggtggagactgttttgaccaatgacatgtaccgtcaaaatgttcgccagatggggcaggaattttcgcagtaccaacctactgagttagctgaacaatacattaatgcactgctgatccaggagaaatcaagccgtttggcagttgtagcctag

SEQ ID NO: 35 - DNA, 1323 bp, Fibrella aestuarina
  Codon optimized GT sequence
atgaatccccagcgcattcttttcgccacgatgcccttcgacggacacttctctccacttactaatttggccgttcacctttcacagctgggacacgacgtccgttggttcgtgggcgggcactacggtcagaaagtaacgcagttagggttacaccactatccctacgtaaaaacccgcaccgttaaccaggagaatctggatcaattgttccctgagcgtgccacaattaaaggcgccattgcccgtattcgtttcgatttaggacaaatctttctgcttcgtgttcctgaacagatcgacgatttgcgtgcgatttatgacgaatggcccttcgatcttatcgtacaagacttggggttcgtcggtggcacatttttacgtgagcttttacccgtgaaagttgtgggggtgggcgtcgtaccgttaactgagtcggatgattgggtaccccctacttcattaggtatgaagccccaatccggtcgcgtgggacgtttagtgtcgcgtcttttaaattatcttgttcaggacgtgatgctgaagcccgctaacgacttacacaatgaattgcgcgcgcagtacggactgcgccccgtgcccggcttcatttttgatgcaactgttcgtcaggcagacttataccttcagagcggggtaccaggatttgaatttcctcgcaaacgcatttcaccgaacgtacgttttatcggacccatgttaccctattcccgcgctaatcgtcaaccatttgaacaggcgatcaaaacacttgcgtacaaacgcgtggtgttggtaactcaaggaacagtagagcgcaacgtcgagaagattatcgttccaacgcttgaggcgtataagaaagatccagataccttagtgatcgtaactaccggtggctcgggtacgcttgcattacgtaaacgttacccacaagctaattttatcattgaagactttattgactttaacgcagtaatgccctacgtcagcgtttacgtaaccaacggcggctatgggggagtcatgttggctttgcagcataaattgcctattgtggccgcgggagtgcatgaagggaagaatgagatcgctgcgcgtattgggtactgtcaggtgggcgtcgatcttcgtaccgagactccgactcccgatcaaattcgtcgtgccgttgctacaattctgggagatgagacttaccgccgccaagtccgtcgtctgagcgacgagttcggtcgctataacccaaaccaacttgcggagcagtatattaacgaattgcttgctcaatcggttggggaacccgttgccgcgctgagctga

SEQ ID NO: 36 - DNA, 1305 bp, Aquimarina macrocephali
  Codon optimized GT sequence
atgacgcgcatgagtcagaagaagattttattcgcttgcattcccgcagacggccattttaatccaatgacggctatcgcaatccatttaaaaaccaagggatacgacgtacgctggtataccggggaggggtataaaaacacgttgcaccgcattggcatcccctatcttcccttccaaaacgcgcaagagctgaagattgaggaaattgacaaaatgtacccggatcgtaagatgttgaagggcattgcacacattaagttcgacatcatcaatttgttcatcaaccgcatgaagggttactatgaggatatcgccgagattcaccaagtttttccatttgatattttggtgtgtgacaatacgttccccgggtccattgttaagaagaagttgaatatccccattgcgtcgatcggagtggtccccctggccttatcagcaccagacttaccgttatacggaattggtcatcagccggctactacgttcttcggaaagcgtaaacaaaattttatcaaacttatggcagacaagttgatcttcgacgaaactaaggttgtatataaccagctgcttcgttccttggatctgtcggaggaggaaaaccttacaatcttcgatattgcccccttacagtctgatgtattcttacagaacggcatccccgagatcgactacccccgctattccttaccagagtccattaagtacgtgggagcgctgcaagtccagactaataacaacaataatcaaaagctgaagaaggattggagcgcgattttggatacatcaaaaaagatcatcctggttagccagggaacagtagaaaaaaacctggacaaacttattatccccagtttagaagcgttcaaagacagcgattatattgtactggtggctacgggttacactgacacaaaaggtttgcaaaaacgttatccgcagcaacacttttatatcgaagatttcattgcctatgacgccgtcatgcctcatattgatgtctttatcatgaacggcggttatggatcggcactgttgagcattaagcatggtgtcccgatgattacggcaggcgtgaatgaggggaaaaacgaaatctgttcacgcatggattattcaggtgttggaatcgacctgaagacagaaaagcctcgtgccgttacaatccaaaacgccacagaacgcattttagggacggacaagtacctggacacgattcagaagattcagcaacgtatgaactcctacaatacattagacatttgcgagcagcacatctcgcgcctgatttcggagtaa

SEQ ID NO: 37 - DNA, 1359 bp, Artificial Sequence
  Codon optimized sequence of Chimera 1
atgaccaaatacaaaaatgagttgacaggcaaacgtattcttttcggtacagttcccggtgatggacactttaacccattaacaggcttagctaaatatttgcaggaattagggtgtgacgtgcgttggtatgcttcggatgtcttcaagtgcaagttagaaaaacttagtatccctcattatggatttaaaaaagcatgggacgttaatggcgtaaatgttaacgaaatcctgcctgaacgtcaaaaattgaccgatcccgctgaaaagttaagtttcgatctgatccatatttttggtaaccgcgcgcccgagtactacgaggacattcttgaaattcatgagagttttccctttgacgtctttattgctgatagttgcttttcggcaattcccttggtgtctaaattgatgagcattccagtagtagcggtcggggtgattcctttggccgaagagtctgtcgatcttgccccatacggtactggattaccgccggcagccacggaagagcaacgtgctatgtactttggcatgaaagatgcacttgcaaacgtcgtgttcaaaactgcaattgacagcttttccgccatcctggaccgctaccaggtgccccatgaaaaggcaatcctgttcgataccttgatccgtcagtccgatcttttccttcaaatcggtgctaaggcttttgaatacgatcgtagcgacttggggaagaatattcgctttattggtagcttacttccttatcagtcgaagaagcaaacgacagcctggagtgacgagcgtttgaaccgctacgagaaaatcgtggtcgtgacccagggaactgttgaaaagaatattgaaaaaatcttagtgccgacattggaggccttccgcgatacggatttgctggtaatcgctacaactggtgggtccggtactgctgagttaaagaaacgttaccctcaggggaacttaattatcgaagatttcatccccttcggagatatcatgccatatgcggatgtctacatcacgaatggagggtacggtggagttatgttgggcattgagaatcaactgccgttagtcgtagcggggatccacgaggggaagaacgaaattaacgcacgcattgggtacttcgagttgggaattaacttaaaaactgaatggcctaagcccgaacaaatgaaaaaggccatcgacgaagtaattggtaacaaaaaatataaggagaacatcacgaaacttgctaaggagttctcaaactaccacccaaacgaattatgcgcacagtacatctctgaagtattgcagaagaccggtcgtctgtacatctcgtcgaagaaggaggaagaaaagatctactaa

SEQ ID NO: 38 - DNA, 1344 bp, Artificial Sequence
  Codon optimized sequence of Chimera 2
atgtccaaccttttttcgtcccagacgaatcttgccagcgtaaaacctttaaaagggcgcaagattctttttgcaaattttcccgccgacgggcatttcaatcctcttacaggcttggccgtacacttacaatggcttgggtgtgacgtgcgttggtatacttcaaacaagtatgccgacaagctgcgtcgtttgaatatcccgcattttccttttcgcaaagccatggacattgctgaccttgagaacatgtttccagagcgcgacgccatcaaggggcaagtcgctaaattgaaattcgatatcattaacgcatttatcctgcgcggtccggagtattacgtagatttgcaggagattcacaagtcatttccattcgatgtcatggttgctgattgcgcctttacaggaattccattcgtcacagacaaaatggatatccccgtggtctcggtaggcgtatttcccttaaccgagaccagcaaagatcttccacccgcagggttggggatcactccatccttctcccttcctggaaagttcaagcaaagcattcttcgctcggttgccgacttagtcttattccgcgaatctaataaagttatgcgcaaaatgttgacggaacatggcattgaccatctttatactaatgtgttcgacttgatggtcaaaaaaagcaccctgttactgcaaagcgggacgccgggttttgaatattaccgcagcgatctgggcaagaatatccgctttatcggctcccttcttccgtatcaatctaagaaacagacaaccgcatggagcgatgagcgtctgaaccgctatgaaaagattgtcgttgtcacccaagggaccgtcgaaaaaaatattgagaaaatcttggttcctaccttagaggcatttcgtgacactgatcttttagtgatcgcaaccacaggtggtagcggaacagcagagttaaaaaagcgctacccccaaggaaatcttatcattgaagatttcattccgtttgacgacgttatgcctcgcgccgatgtatacgtgactaatggaggatacggaggtacgttactgtctatccataatcagctgccaatggtcgccgccggcgttcacgaaggcaagaatgaagtatgttcccgtattgggcattttggatgtggaatcaatttggaaaccgaaaccccaacccctgaccagattcgcgagtcagttcacaaaatcttgtcaaatgacatcttcaagaagaatgtattccgcattagcacacatttggatgtcgatgcgaacgagaaaagcgccgggcacattttagacttactggaagaacgcgtagtatgtggataa

SEQ ID NO: 39 - DNA, 29 bp, Artificial Sequence
  GTC-Ndel-for
catatgagtaatttattttcttcacaaac

SEQ ID NO: 40 - DNA, 27 bp, Artificial Sequence
  GTC-BamHI-rev
ggatccttagtatatcttttcttcttc

SEQ ID NO: 41 - DNA, 28 bp, Artificial Sequence
  GTF_XhoI_for
ctcgagatgacgaaatacaaaaatgaat

SEQ ID NO: 42 - DNA, 25 bp, Artificial Sequence
  GTF_BamHI_rev
ggatccttaaccgcaaacaacccgc

SEQ ID NO: 43 - DNA, 29 bp, Artificial Sequence
  GTL_XhoI_for
ctcgagatgacaactaaaaaaatcctgtt

SEQ ID NO: 44 - DNA, 26 bp, Artificial Sequence
  GTL_BamHI_rev
ggatccttagattgcttctacggctt

SEQ ID NO: 45 - protein, 20 aa, Artificial Sequence
  partial sequence of SEQ ID NO. 1
  VARIANT 1: Lys = Arg
  UNSURE 6..7: Xaa = any amino acid
  UNSURE 9: Xaa = any amino acid
  VARIANT 14: Asn = Ser
  UNSURE 18: Xaa = any amino acid
  VARIANT 19: Leu = Ile
KILFAXXPXDGHFNPLTXLA

SEQ ID NO: 46 - protein, 7 aa, Artificial Sequence
  partial sequence of SEQ ID NO. 1
  UNSURE 2: Xaa = any amino acid
  VARIANT 7: Tyr = Phe
GXDVRWY

SEQ ID NO: 47 - protein, 4 aa, Artificial Sequence
  partial sequence of SEQ ID NO 1
  VARIANT 1: Phe = Tyr or Leu
  VARIANT 3: Glu = Asp
FPER

SEQ ID NO: 48 - protein, 17 aa, Artificial Sequence
  partial sequence of SEQ ID NO 1
  UNSURE 3: Xaa = Ala, Ile, Leu, Met, Phe, Pro or Val
  UNSURE 4..5: Xaa = any amino acid
  UNSURE 6: Xaa = Ala, Ile, Leu, Met, Phe, Pro or Val
  UNSURE 8..9: Xaa = any amino acid
  UNSURE 11..12: Xaa = any amino acid
  VARIANT 14: Tyr = Phe
  UNSURE 15: Xaa = Ala, Ile, Leu, Met, Phe, Pro or Val
  UNSURE 16: Xaa = any amino acid
FDXXXXFXXRXXEYXXD

SEQ ID NO: 49 - protein, 17 aa, Artificial Sequence
  partial sequence of SEQ ID NO. 1
  VARIANT 1: Phe = Trp
  UNSURE 4: Xaa = any amino acid
  UNSURE 5..7: Xaa = Ala, Ile, Leu, Met, Phe, Pro or Val
  UNSURE 8: Xaa = any amino acid
  VARIANT 9: Asp = Glu
  UNSURE 10..11: Xaa = any amino acid
  UNSURE 13..16: Xaa = any amino acid
FPFXXXXXDXXFXXXXF

SEQ ID NO: 50 - protein, 25 aa, Artificial Sequence
  partial sequence of SEQ ID NO 1
  UNSURE 3: Xaa = any amino acid
  UNSURE 5: Xaa = Asn, Cys, Gln, Gly, Ser, Thr or Tyr
  UNSURE 6..8: Xaa = any amino acid
  VARIANT 10: Phe = Ala
  UNSURE 12: Xaa = any amino acid
  UNSURE 14: Xaa = any amino acid
  UNSURE 16..17: Xaa = any amino acid
  UNSURE 19..23: Xaa = any amino acid
  VARIANT 25: Lys = Arg
PLXEXXXXLPPXGXGXXPXXXXXGK

SEQ ID NO: 51 - protein, 12 aa, Artificial Sequence
  partial sequence of SEQ ID NO. 1
  UNSURE 3: Xaa = any amino acid
  UNSURE 5: Xaa = any amino acid
  UNSURE 6: Xaa = Phe or Lys
  UNSURE 11: Xaa = any amino acid
LQXGXXGFEYXR

SEQ ID NO: 52 - protein, 21 aa, Artificial Sequence
  partial sequence of SEQ ID NO 1
  UNSURE 5: Xaa = Ala, Ile, Leu, Met, Phe, Pro, Trp or Val
  VARIANT 7: Lys = Arg
  UNSURE 8..10: Xaa = any amino acid
  UNSURE 12..14: Xaa = Ala, Ile, Leu, met, Phe, Pro, Trp or Val
  VARIANT 21: Arg = Lys
TQGTXEKXXXKXXXPTLEAFR

SEQ ID NO: 53 - protein, 8 aa, Artificial Sequence
  partial sequence of SEQ ID NO 1
  UNSURE 3..4: Xaa = Ala, Ile, Leu, Met, Phe, Pro, Trp or Val
LVXXTTGG

SEQ ID NO: 54 - protein, 47 aa, Artificial Sequence
  partial sequence of SEQ ID NO 1
  VARIANT 2: Glu = Asp
  UNSURE 8..9: Xaa = any amino acid
  VARIANT 10: Val = Ile
  UNSURE 13..14: Xaa = any amino acid
  VARIANT 17: Tyr = Phe
  VARIANT 18: Ile = Val
  VARIANT 19: Thr = Ser
  VARIANT 23: Tyr = Phe
  VARIANT 27: Met = Leu
  UNSURE 29: Xaa = any amino acid
  UNSURE 31: Xaa = any amino acid
  VARIANT 32: Asn = His
  UNSURE 33: Xaa = any amino acid
  UNSURE 36: Xaa = Ala, Ile, Leu, Met, Phe, Pro, Trp or Val
  UNSURE 38: Xaa = any amino acid
  UNSURE 41: Xaa = Ala, Ile, Leu, Met, Phe, Pro, Trp or Val
IEDFIPFXXVMPXXDVYITNGGYGGVMLXIXNXLPXVXAGXHEGKNE

SEQ ID NO: 55 - protein, 6 aa, Artificial Sequence
  partial sequence of SEQ ID NO. 1
HEGKNE

SEQ ID NO: 56 - protein, 464 aa, Artificial Sequence
  Chimera 1 frameshift
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKQAGFISAVKRKKKRYTKDPAANKARKEA

SEQ ID NO: 57 - DNA, 1395 bp, Artificial Sequence
  Chimera 1 frameshift
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaacaggcaggctttatatcagcagtaaaaaggaagaagaaaagatatactaaggatccggctgctaacaaagcccgaaaggaagcgtag

SEQ ID NO: 58 - protein, 452 aa, Artificial Sequence
  Chimera 3
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGENVRFVGALLPYSESKSRQPWFDQKLLQYGRIVLVTQGTVEHDINKILVPTLEAFKNSETLVIATTGGNGTAELRARFPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 59 - DNA, 1359 bp, Artificial Sequence
  Chimera 3
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagcgacctgggcgaaaatgtccgttttgtcggcgcattgctgccgtactcggaaagtaaatcccggcagccctggtttgatcagaaacttttacaatatggcaggattgtgctggttacccagggcactgttgagcacgatatcaacaagatacttgtacccacgctggaagctttcaaaaattctgagacgctggtaattgccacaacaggcggtaatgggacagcggaattgcgcgcgcgttttcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 60 - DNA, 1359 bp, Artificial Sequence
  Codon-optimized nucleotide sequence of Chimera      3 (optimized for E. coli)
atgaccaaatacaaaaatgagttgaccggcaaacgtattttgtttggaaccgtgcctggagatggacatttcaaccccttaacaggcttagccaagtacctgcaagaactgggctgcgatgtacgctggtatgcatctgatgtatttaagtgcaaactggagaagctgagcatccctcactatgggttcaagaaggcttgggatgtaaatggagtaaatgttaatgaaattcttccggagcgtcaaaagctgaccgaccctgcggaaaagctgagtttcgaccttatccacatttttggaaatcgcgctcctgaatattacgaggacatcttggaaattcacgagagttttcctttcgacgtcttcatcgccgactcctgcttcagtgctattcccttagtttccaagcttatgtctattcctgtcgtggcagtaggggtgatcccgctggcagaagagagtgtggacttagcaccatacggaactggcctgccgccagctgcgacagaagagcagcgcgccatgtatttcggcatgaaggacgcacttgccaacgtggtgttcaaaacagccattgactcgttttccgccattttagatcgttatcaagtgcctcacgagaaagcgatcttatttgatactcttattcgtcaaagcgatttgtttttgcaaatcggagccaaagctttcgagtatgaccgcagcgatttgggggaaaacgtgcgtttcgttggagccctgctgccttattcggagagcaaaagtcgtcaaccctggttcgatcaaaagttgttacaatatgggcgcattgtcttggtcactcaggggacggtggaacatgatattaataagattctggttcctactttagaggcatttaaaaactcggaaaccctggtcatcgcgacaacaggaggaaatggtacagcagaattacgtgcgcgctttcctcagggcaacttaatcattgaggacttcattccatttggtgacattatgccatacgctgatgtatatatcaccaatggtggttacggcggagttatgcttggcatcgaaaatcaactgccccttgtcgtagccggcatccacgaaggaaagaacgagatcaacgcacgtattgggtactttgagcttggaatcaatctgaaaacggagtggccgaagccagagcagatgaaaaaagcgattgacgaagttatcggtaataagaagtacaaagagaatatcacaaaactggcgaaggaattctcaaactaccatcctaacgaattgtgcgcccaatacatctctgaagtcttacagaagaccggccgcttgtacatttcgtccaagaaggaagaagaaaagatttactaa

SEQ ID NO: 61 - protein, 452 aa, Artificial Sequence
  Chimera 4
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGENVRFVGALLPYSESKSRQPWFDQKLLQYGQIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 62 - DNA, 1359 bp, Artificial Sequence
  Chimera 4
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagcgacctgggcgaaaatgtccgttttgtcggcgcattgctgccgtactcggaaagtaaatcccggcagccctggtttgatcagaaacttttacaatatggcaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 63 - DNA, 1362 bp, Artificial Sequence
  Codon-optimized nucleotide sequence of chimera      4 (optimized for E. coli)
atgaccaaatacaaaaatgagttgaccggcaaacgtattttgtttggaaccgtgcctggagatggacatttcaaccccttaacaggcttagccaagtacctgcaagaactgggctgcgatgtacgctggtatgcatctgatgtatttaagtgcaaactggagaagctgagcatccctcactatgggttcaagaaggcttgggatgtaaatggagtaaatgttaatgaaattcttccggagcgtcaaaagctgaccgaccctgcggaaaagctgagtttcgaccttatccacatttttggaaatcgcgctcctgaatattacgaggacatcttggaaattcacgagagttttcctttcgacgtcttcatcgccgactcctgcttcagtgctattcccttagtttccaagcttatgtctattcctgtcgtggcagtaggggtgatcccgctggcagaagagagtgtggacttagcaccatacggaactggcctgccgccagctgcgacagaagagcagcgcgccatgtatttcggcatgaaggacgcacttgccaacgtggtgttcaaaacagccattgactcgttttccgccattttagatcgttatcaagtgcctcacgagaaagcgatcttatttgatactcttattcgtcaaagcgatttgtttttgcaaatcggagccaaagctttcgagtatgaccgcagcgatttgggggaaaacgtgcgtttcgttggagccctgctgccttattcggagagcaaaagtcgtcaaccctggttcgatcaaaagttgttacaatatgggcgcaaaatcgtcgtagtcactcagggaactgtagagaaaaacatcgaaaagattttggtgccaacccttgaggctttccgcgacactgacctgcttgtgatcgcgacgacgggaggttcaggaaccgctgaattgaaaaaacgttaccctcagggcaacttaatcattgaggacttcattccatttggtgacattatgccatacgctgatgtatatatcaccaatggtggttacggcggagttatgcttggcatcgaaaatcaactgccccttgtcgtagccggcatccacgaaggaaagaacgagatcaacgcacgtattgggtactttgagcttggaatcaatctgaaaacggagtggccgaagccagagcagatgaaaaaagcgattgacgaagttatcggtaataagaagtacaaagagaatatcacaaaactggcgaaggaattctcaaactaccatcctaacgaattgtgcgcccaatacatctctgaagtcttacagaagaccggccgcttgtacatttcgtccaagaaggaagaagaaaagatttactaa

SEQ ID NO: 64 - DNA, 38 bp, Artificial Sequence
  GTSopt_pET_fw
gggaattccatatgatgaaatatatcagctccattcag

SEQ ID NO: 65 - DNA, 33 bp, Artificial Sequence
  GTSopt_pET_rv
cgggatccttaaaccagaacttcggcctgatag

SEQ ID NO: 66 - DNA, 59 bp, Artificial Sequence
  Bridge_P1_pETGTD
gcggccatatcgacgacgacgacaagcatatgacgaaatacaaaaatgaattaacaggt

SEQ ID NO: 67 - DNA, 51 bp, Artificial Sequence
  Bridge_P1_pETGTD
ggaagaagaaaagatatactaaggatccggctgctaacaaagcccgaaagg

SEQ ID NO: 68 - DNA, 26 bp, Artificial Sequence
  Chim_P1_D_Nde_for
catatgacgaaatacaaaaatgaatt

SEQ ID NO: 69 - DNA, 20 bp, Artificial Sequence
  Chim_P1_D_rev
gcggtcatactcaaatgatt

SEQ ID NO: 70 - DNA, 21 bp, Artificial Sequence
  Chim_P1_C_for
agtgatctgggaaaaaatatc

SEQ ID NO: 71 - DNA, 29 bp, Artificial Sequence
  Chim_P1_C_Bam_rev
ggatccttagtatatcttttcttcttcct

SEQ ID NO: 72 - DNA, 33 bp, Artificial Sequence
  GTDopt_pEt_fw
gggaattccatatgatgaccaaatacaaaaatg

SEQ ID NO: 73 - DNA, 33 bp, Artificial Sequence
  Chim3_pET_rv
cgggatccttagtaaatcttttcttcttccttc

SEQ ID NO: 74 - DNA, 28 bp, Artificial Sequence
  1r-Chim3-opt-o(Chim3-opt)
tgccctgaggaaagcgcgcacgtaattc

SEQ ID NO: 75 - DNA, 28 bp, Artificial Sequence
  2f-Chim3-opt-o(Chim3-opt)
tgcgcgctttcctcagggcaacttaatc

SEQ ID NO: 76 - DNA, 40 bp, Artificial Sequence
  1f-Assembly-o(Vec)
tgacgataaggatcgatggggatccatgaccaaatacaaa

SEQ ID NO: 77 - DNA, 43 bp, Artificial Sequence
  1r-Assembly-o(Vec)
tatggtaccagctgcagatctcgagttagtaaatcttttcttc

SEQ ID NO: 78 - DNA, 32 bp, Artificial Sequence
  1r-Chim4_GTD-o(Chim4_GTC)
cgattttgcgcccatattgtaacaacttttga

SEQ ID NO: 79 - DNA, 28 bp, Artificial Sequence
  2f-Chim4_GTC-o(Chim4_GTD)
acaatatgggcgcaaaatcgtcgtagtc

(19) United States
(12) Patent Application Publication
(10) Pub. No.: US 2019/0100549 A1
(43) Pub. Date: Apr. 4, 2019

(54) FLAVONOIDE-TYPE COMPOUNDS BEARING AN O-RHAMNOSYL RESIDUE

(71) Applicant: Universitaet Hamburg, Hamburg (DE)

(72) Inventors: Ulrich Rabausch, Hamburg (DE); Henning Rosenfeld, Hamburg (DE)

(21) Appl. No.: 16/069,738

(22) PCT Filed: Jan. 13, 2017
(86) PCT No.: PCT/EP2017/050678

§ 371 Date: Jul. 12, 2018

(30) Foreign Application Priority Data
Jan. 15, 2016 (DE) 16151613.3

(51) Int. Cl. C07H 17/07

(52) U.S. Cl. CPC C07H 17/07

(57) ABSTRACT

The present invention relates to compounds of formula (II)

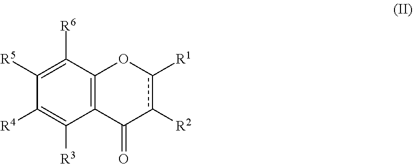

useful in the treatment of many diseases such as a skin disease, an allergy, an autoimmune disease, a cardiovascular disease, a lung disease, asthma, a bacterial, viral or parasitic disease, metabolic syndrome, cancer, Alzheimer's disease or diabetes and are furthermore useful in the preparation of cosmetics and for use in food and animal feed.

### FIELD OF THE INVENTION

The present invention relates to flavonoid-type compounds which bear an O-rhamnosyl-containing residue and to the pharmaceutical and non-pharmaceutical as well as cosmetic and non-cosmetic use thereof as well as to compositions comprising these compounds.

It is an object of the present invention to provide novel flavonoid-type compounds with increased solubility, bioavailability, stability, improved pharmacological profile and/or flavor enhancing or modulating activities.

### BACKGROUND OF THE INVENTION

Flavonoids are a class of polyphenol compounds which are commonly found in a large variety of plants. Flavonoids comprise a subclass of compounds such as anthoxanthins, flavanones, flavanonols, flavans and anthocyanidins etc. Flavonoids are known to possess a multitude of beneficial properties which make these compounds suitable for use as antioxidants, anti-inflammatory agents, anti-cancer agents, antibacterials, antivirals, antifungals, antiallergenes, and agents for preventing or treating cardiovascular diseases. Furthermore, some flavonoids have been reported to be useful as flavor enhancing or modulating agents.

Due to this wide variety of possible applications, flavonoids are compounds of high importance as ingredients in cosmetics, food, drinks, nutritional and dietary supplements, pharmaceuticals and animal feed. However, use of these compounds has often been limited due to the low water solubility, low stability and limited availability. A further factor which has severely limited use of these compounds is the fact that only a few flavonoids occur in significant amounts in nature while the abundance of other flavonoids is nearly negligible. As a result, many flavonoids and their derivatives are not available in amounts necessary for large-scale industrial use.

Glycosylation is one of the most abundant modifications of flavonoids, which has been reported to significantly modulate the properties of these compounds. For example, glycosylation may lead to higher solubility and increased stability, such as higher stability against radiation or temperature. Furthermore, glycosylation may modulate pharmacological activity and bioavailability of these compounds.

Glycosylated derivatives of flavonoids occur in nature as O-glycosides or C-glycosides, while the latter are much less abundant. Such derivatives may be formed by the action of glycosyl transferases (GTases) starting from the corresponding aglycones.

However, flavonoids constitute the biggest class of polyphenols in nature (Ververidis (2007) Biotech. J. 2(10):1214-1234). The high variety of flavonoids originates from addition of various functional groups to the ring structure. Herein, glycosylation is the most abundant form and the diversity of sugar moieties even more leads to a plethora of glycones.

But in nature only some flavonoid glycones prevail. As described above, among these are the 3-O-β-D-glucosides, e.g. isoquercitrin, the flavonoid-7-β-D-glucosides, e.g. genistin, and the 3- and 7-rhamnoglucosides, e.g. rutin and naringin. Generally, glucosides are the most frequent glycosidic forms with 3- and 7-O-β-D-glucosides dominating. In contrast, glycosides concerning other sugar moieties, e.g. rhamnose, and other glycosylation positions than C3 and C7 rarely occur and are only present in scarce quantities in specific plant organs. This prevents any industrial uses of such compounds. For example, De Bruyn (2015) Microb Cell Fact 14:138 describes methods for producing rhamnosylated flavonoids at the 3-O position. Also, 3-O rhamnosylated versions of naringenin and quercetin are described by Ohashi (2016) Appl Microbiol Biotechnol 100:687-696. Metabolic engineering of the 3-O rhamnoside pathway in E. coli with kaempferol as an example is described by Yang (2014) J Ind Microbiol Biotech 41:1311-18. Finally, the in vitro production of 3-O rhamnosylated quercetin and kaempferol is described by Jones (2003) J Biol Chem 278:43910-18. None of these documents describes or suggests the production of 5-O rhamnosylated flavonoids.

Examples of naturally occurring O-glycosides are quercetin-3-O-β-D-glucoside (Isoquercitrin) and genistein-7-O-β-glucoside (Genistin).

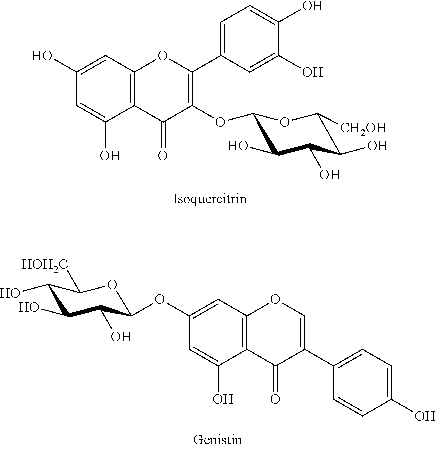

In contrast, the corresponding 5-O-glycosides are found very rarely in nature. In particular, the 5-O-rhamnosides are virtually unknown with the three exceptions being a naringenin-5-O-α-L-rhamnoside which has been reported to be contained in extracts from the stem of Prunus cerasoides Roxb., eriodictyol-5-O-α-L-rhamnopyranoside from the medicinal plant Cleome viscosa, and taxifolin-3,5-di-O-α-L-rhamnopyranoside (Shrivastava et al., Indian J. Chem 1982, 21B, 406-407, Chauhan et al., Planta Med 1977 32(07):217-222, Srivastava and Srivastava 1979 Phytochemistry 18:2058-2059).

WO 2014/191524 relates to enzymes catalyzing the glycosylation of polyphenols, in particular flavonoids, benzoic acid derivatives, stilbenoids, chalconoids, chromones, and coumarin derivatives.

U.S. Pat. No. 5,587,176 relates to the field of sebum control and treatment of acne in mammalian skin and scalp, in particular, to methods for sebum control and treatment of acne, and related pilosebaceous disorders, in human skin and scalp. Compositions disclosed therein contain hesperetin.

EP 2 220 945 relates to an aroma composition for reducing or suppressing an unpleasant (taste) impression in the oral cavity, comprising (i) one or more sweeteners including their physiologically tolerated salts, which may be dihydroquercetin-3-acetate, and (ii) one or more bitter-masking aroma substances and/or flavorings.

Compositions containing hesperetin for enhancing the sweet taste of a sweet-tasting substance or the sweet olfactory impression of a flavoring which gives a sweet olfactory impression are described in EP 1 909 599.

WO 2009/031106 discloses the cosmetic use of at least an effective amount of hesperidin or of one of its derivatives in combination with at least an effective amount of a least one microorganism, in particular probiotic microorganism, or one of its fractions as agent for preventing a reduction in and/or reinforcing the barrier function of the skin.

U.S. Pat. No. 6,521,668 discloses a cosmetic composition comprising an antioxidant selected from the group consisting of: hesperetin, tetrahydrocurcumin, tetrahydrodemethoxycurcumin, tetrahydrobisdemethoxycurcumin, and mixtures thereof and a cosmetically acceptable carrier.

WO 2005/070383 relates to a skin lightening product comprising components (a) a flavanoid, (b) vitamin C and (c) vitamin E wherein at least component (b) is provided in a form suitable for systemic administration with the other components being provided in a form suitable for topical administration.

US 2010/0190727 relates to the use, especially the cosmetic use, of at least one monosaccharide chosen from mannose, rhamnose and a mixture thereof, for reducing or preventing the signs of ageing of the skin or its integuments.

EP 2 027 279 relates to phenolics derivatives which were obtained by enzymatic condensation of phenolics selected among pyrocatechol or its derivatives including (i) protocatechuic acid and its derivatives, (ii) 3,4-dihydroxycinnamic acid with its trans isomer or caffeic acid and its derivatives, especially hydrocaffeic acid, rosmarinic acid, chlorogenic acid and caffeic acid phenethyl ester, and with its cis-isomer and its derivatives, especially esculin, (iii) dihydroxyphenylglycol, and (iv) members of the flavonoid family such as taxifolin and fustin (dihydroflavonols), fisetin (a flavonol), eriodictyol (a flavanone), with the glucose moiety of sucrose.

WO 2006/094601 relates to chromen-4-one derivatives, the production thereof, and the use of the same for the care, preservation or improvement of the general state of the skin or especially the hair, and for the prophylaxis of time-induced and/or light-induced ageing processes of the human skin or especially human hair.

The use of chromen-4-one derivatives to prevent, reduce or combat signs of cellulite and/or reduce localized fatty excesses is described in WO 2008/025368.

WO 2006/045760 discloses the use of specific glycosylated flavanones as agents for the browning of skin and/or hair in vivo.

EP 0 774 249 discloses cosmetic compositions containing combinations of flavanones: eriodictyol and/or taxifolin combined with taxifolin and/or hesperetin. Alternatively, a flavanone is combined with a short-chain lipid. The compositions are reported to enhance keratinocyte differentiation in skin, thus decreasing skin dryness and decreasing appearance of wrinkles.

A compendium series on the isolation and characterization of flavonoids has been published under the title THE FLAVONOIDS: Advances in Research by Harborne and Williams.

Ohguchi et al. have reported on the stimulation of melanogenesis by the citrus flavonoid naringenin in mouse B16 melanoma cells (Biosci. Biotechnol. Biochem. 2006, 70(6), 1499-1501). Melamin contents and tyrosinase activities as well as expression levels of melanogenic enzymes are reported to have been increased by naringenin.

A naringenin-4′-O-alpha-L-rhamnopyranoside has been reported by Yadava et al. as having been isolated from the stem of Crotalaria striata DC. (Journal of the Indian Chemical Society 1997, 74(5), 426-427).

Goodenowe et al. reported on the integrated analysis of metabolome and transcriptome of Arabidopsis plants over-expressing an MYB transcription factor (The Plant Journal 2005, 42(2), 218-235). Two putative glycosyltransferase genes (At5g17050 and At4g14090) induced by PAP1 expression were confirmed to encode flavonoid 3-O-glucosyltransferase and anthocyanin 5-O-glucosyltransferase, respectively, from the enzymatic activity of their recombinant proteins in vitro and results of the analysis of anthocyanins in the respective T-DNA-inserted mutants.

Cavia-Saiz et al. published a comparative study on the antioxidant properties, radical scavenging activity and biomolecule protection capacity of flavonoid naringenin and its glycoside naringin (J. Sci. Food Agric. 2010, 90, 1238-1244).

Shimoda K and Hamada H reported on the production of hesperetin glycosides by Xanthomonas campestris and cyclodextrin glucanotransferase and their anti-allergic activities (Nutrients 2010, 2(2):171-180).

Chauhan et al. reported on the isolation of a hesperetin-7-rhamnoside from Cordia obliqua (Phytochemistry 1978, 17(2), 334).

Xie et al. published a study concerning the role of highly conserved residues forming the acceptor binding pocket of the promiscuous glycosyltransferase MGT in defining the specificity towards a panel of flavonoids (Biochemistry (Mosc) 2013, 78(5), 536-541).

The preparation and taste of certain glycosides of flavanones and of dihydrochalcones has been published by Sachiko Esaki et al. (Biosci. Biotech. Biochem. 1994, 58(8), 1479-1485).

Laslo Janvary et al. found that a double mutation in the anthocyanin 5-O-glucosyltransferase gene disrupts enzymatic activity in Vitis vinifera L (J Agric Food Chem 57(9), 3512-3518).

Daimon et al. reported that the silkworm Green b locus encodes a quercetin 5-O-glucosyltransferase that produces green cocoons with UV-shielding properties (Proc Natl Acad Sci USA 2010, 107(25), 11471-11476).

### SUMMARY OF THE INVENTION

It is an object of the present invention to provide novel flavonoid-type compounds with increased solubility, bioavailability, stability, improved pharmacological profile and/or flavor enhancing or modulating activities. Accordingly, the present invention provides flavonoid-type compounds of formula (I) which contain a rhamnosyl containing residue at a position which has so far not been synthetically accessible for rhamnosylation.

This novel flavonoid-type compounds can

stimulate and improve skin and hair follicle biology and thereby

affect skin and hair pigmentation, i.e. pro-pigmenting or depigmenting effects

regenerate hair growth and hair follicle constitution

reduce wrinkle depth of skin, e.g. increase or decrease levels of metalloproteinases as collagenases, gelatinases

improve skin blood circulation and supplementation

optimize wound healing

reduce inflammatory processes

protect the skin from environmental pollution, xenobiotica, UV irradiation, and IR-irradiation

maintain cell homeostasis

have radical scavenging and antioxidant activities

alter blood pressure and stabilize vascular constitution

modify the taste impression of food, drinks, food supplements, and pharmaceuticals, e.g. sweetening effect or reduce astringent taste or lingering effects

have antibacterial activity

have antiviral capacity

have antifungal activity

have a cancer, diabetes and obesity preventing effect

have a less coloring/staining effect on formulations and compositions

Accordingly, the present invention provides a compound of the following formula (I)

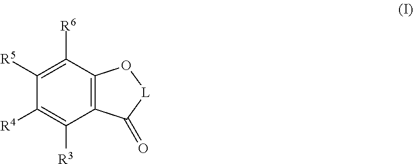

wherein

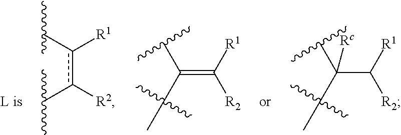

[symbol] is a double bond or a single bond;
R¹ and R² are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from —OH; or R¹ and R² are joined together to form, together with the carbon atom(s) that they are attached to, a carbocyclic or heterocyclic ring being optionally substituted with one or more substituents R^{e}; wherein each R^{e} is independently selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.
R⁴, R⁵ and R⁶ are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.
Alternatively, R⁴ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and R⁵ and R⁶ are joined together to form, together with the carbon atoms that they are attached to, a carbocyclic or heterocyclic ring being optionally substituted with one or more substituents R^{c}.
Alternatively, R⁴ and R⁵ are joined together to form, together with the carbon atoms that they are attached to, a carbocyclic or heterocyclic ring being optionally substituted with one or more substituents R^{c}; and R⁶ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.
Each R^{a} is independently selected from a single bond, C₁₋₅ alkylene, C₂₋₅ alkenylene, arylene and heteroarylene; wherein said alkylene, said alkenylene, said arylene and said heteroarylene are each optionally substituted with one or more groups R^{c}.
Each R^{b} is independently selected from hydrogen, C₁₋₅ alkyl, C₂₋₅ alkenyl, C₂₋₅ alkynyl, heteroalkyl, cycloalkyl, heterocycloalkyl, aryl and heteroaryl; wherein said alkyl, said alkenyl, said alkynyl, said heteroalkyl, said cycloalkyl, said heterocycloalkyl, said aryl and said heteroaryl are each optionally substituted with one or more groups R^{c}.
Each R^{c} is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.
R³ is —O-(rhamnosyl) wherein said rhamnosyl is optionally substituted at one or more of its —OH groups with one or more groups independently selected from C₁₋₅ alkyl, C₂₋₅ alkenyl, C₂₋₅ alkynyl, a monosaccharide, a disaccharide and an oligosaccharide.
Each R^{d} is independently selected from a monosaccharide, a disaccharide and an oligosaccharide.

### DESCRIPTION OF THE FIGURES

FIG. 1: Determination of solubility of naringenin-5-O-α-L-rhamnoside (NR1) in water. Defined concentrations of NR1 were 0.22 μm-filtered before injection to HPLC. Soluble concentrations were calculated from peak areas by determined regression curves.

FIG. 2: HPLC-chromatogram of naringenin-5-O-α-L-rhamnoside

FIG. 3: HPLC-chromatogram of naringenin-4′-O-α-L-rhamnoside

FIG. 4: HPLC-chromatogram of prunin (naringenin-7-O-β-D-glucoside)

FIG. 5: HPLC-chromatogram of homoeriodictyol-5-O-α-L-rhamnoside (HEDR1)

FIG. 6: HPLC-chromatogram of HEDR3 (4:1 molar ratio of homoeriodictyol-7-O-α-L-rhamnoside and homoeriodictyol-4′-O-α-L-rhamnoside)

FIG. 7: HPLC-chromatogram of homoeriodictyol-4′-O-β-D-glucoside (HED4′Glc)

FIG. 8: HPLC-chromatogram of hesperetin-5-O-α-L-rhamnoside (HESR1)

FIG. 9: HPLC-chromatogram of hesperetin-3′-O-α-L-rhamnoside (HESR2)

FIG. 10: UV₂₅₄-chromatogram of hesperetin bioconversion 141020, sample injection volume was 1.2 L applied by the pumping system

FIG. 11: ESI-TOF negative mode MS-analysis of fraction 3 from hesperetin bioconversion 141020

FIG. 12: ESI-TOF negative mode MS-analysis of fraction 6 from hesperetin bioconversion 141020

FIG. 13: prepLC UV₂₅₄-chromatogram of PFP-HPLC of fraction 3 bioconversion 141020; the main peak (HESR1) between 3.1 min and 3.5 min was HESR1.

FIG. 14: ESI-TOF negative mode MS-analysis of fraction 3 from 140424_Naringenin-PetC

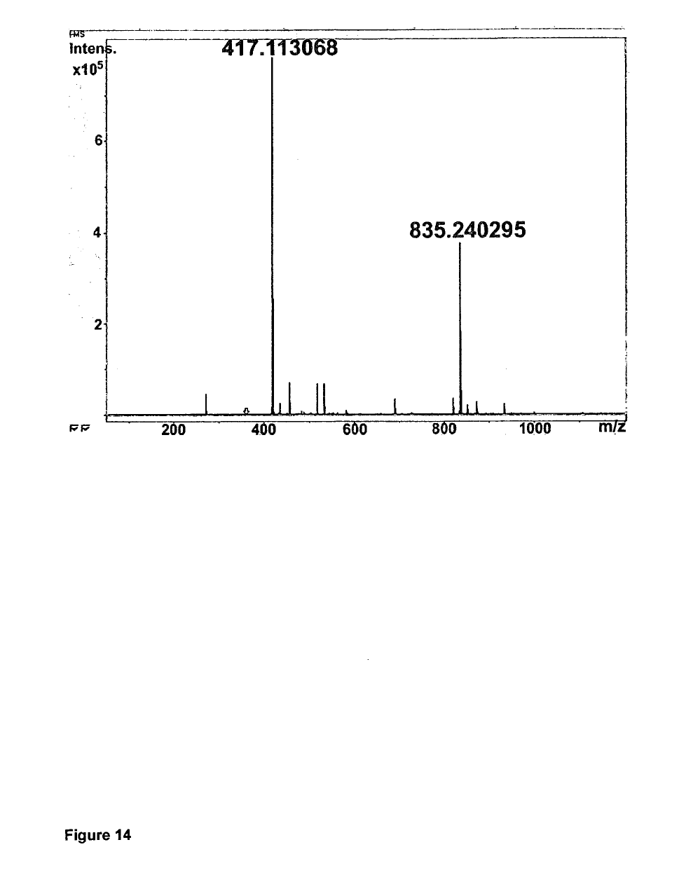

FIG. 15: ESI-TOF negative mode MS-analysis of fraction 5 from 140424_Naringenin-PetC

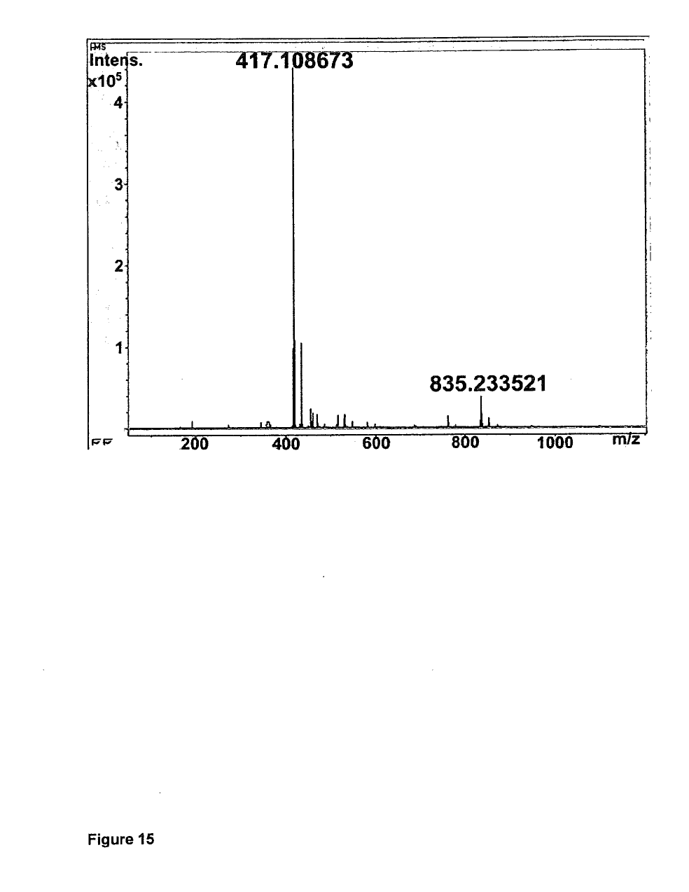

FIG. 16: UV-chromatogram of conversion after 24 h in bioreactor unit 1 150603_Naringenin-PetC

FIG. 17: UV₃₃₀ chromatogram of an extract from a naringenin biotransformation with PetD

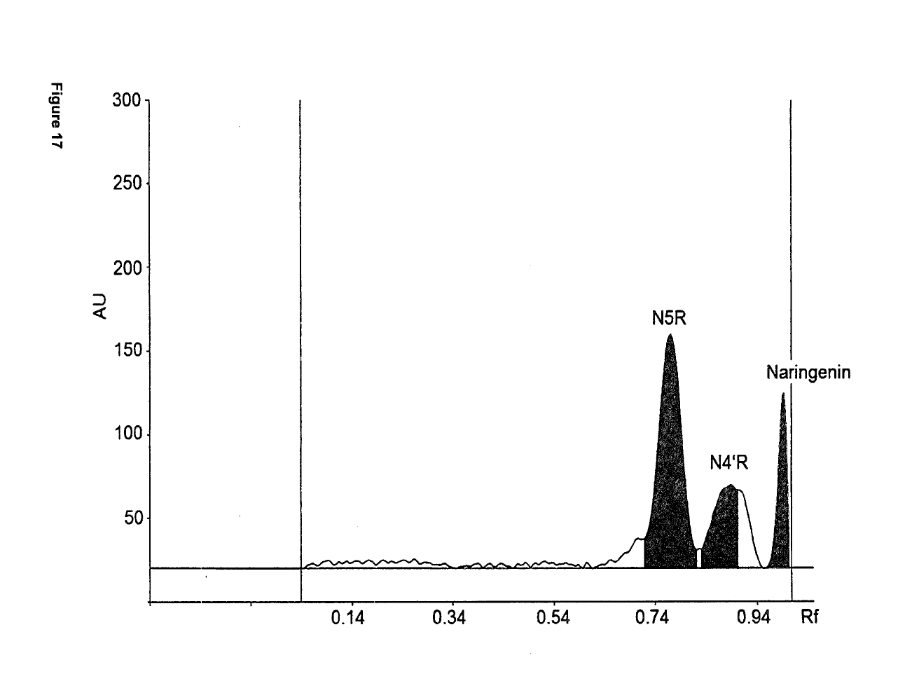

FIG. 18: UV₃₃₀ chromatogram of an extract from a naringenin biotransformation with PetC

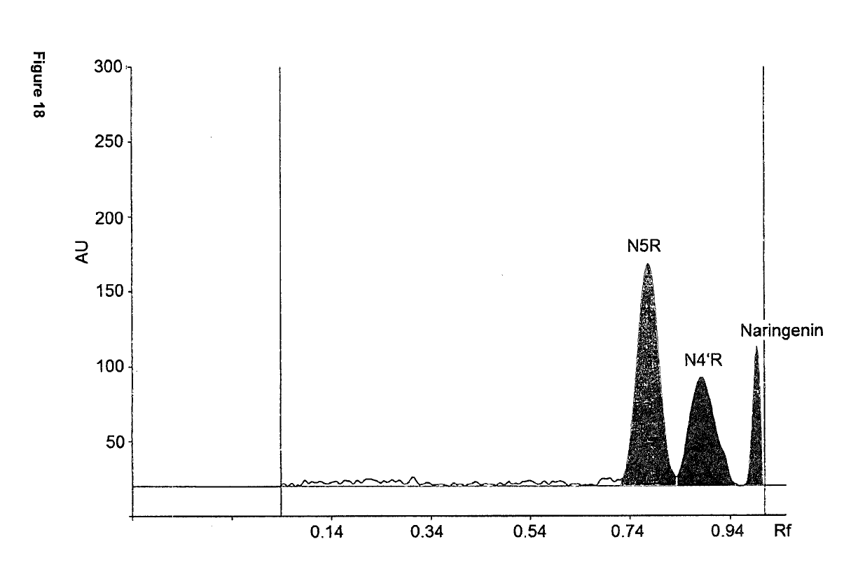

FIG. 19: UV 210-400 nm absorbance spectra of N5R peaks from figures U1 (middle) and U2 (dark) vs. prunin, the naringenin-7-O-β-D-glucoside (light).

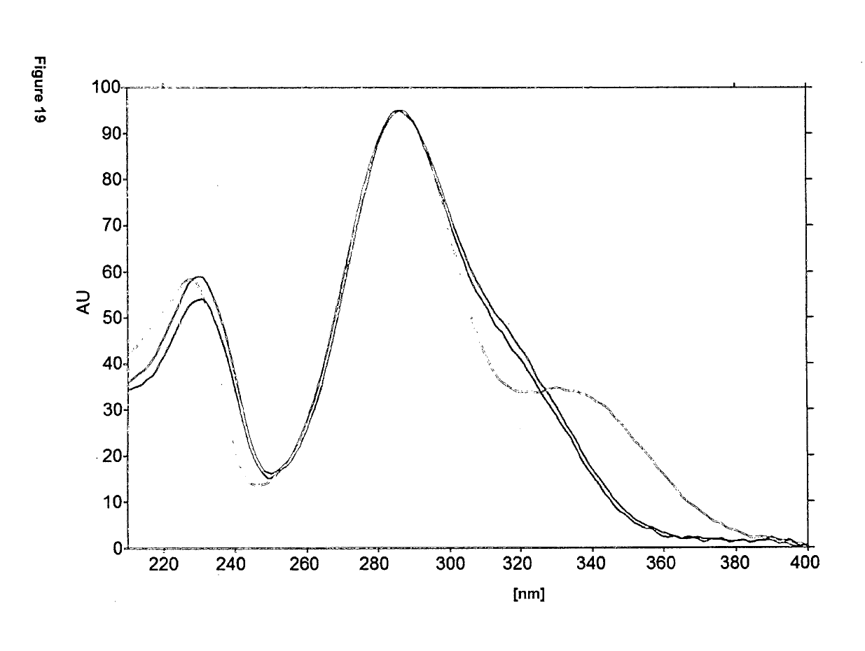

FIG. 20: UV 210-400 nm absorbance spectra of GTF product peak Rf 0.77 (dark) vs. prunin (light).

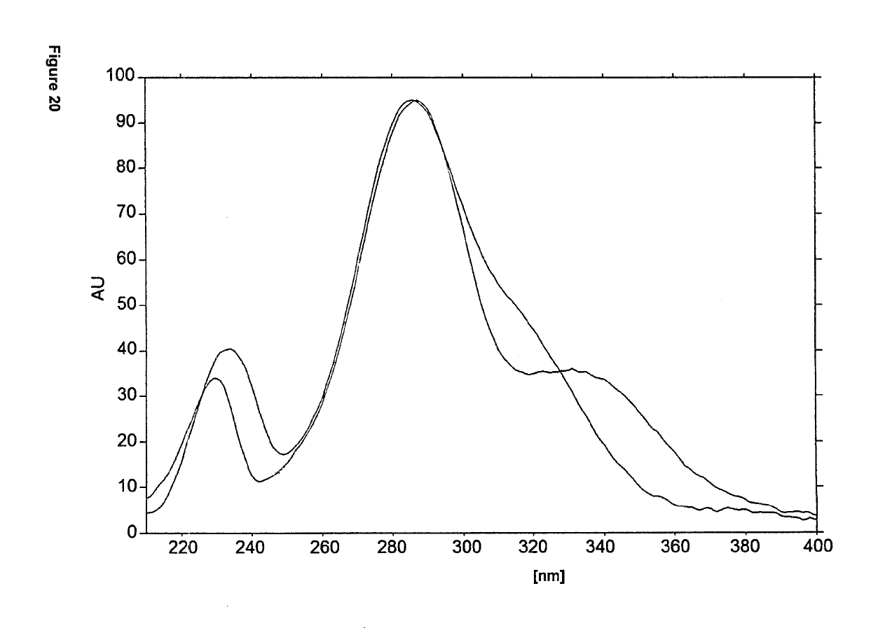

FIG. 21: UV₃₃₀ chromatogram of an extract from a naringenin biotransformation with PetF

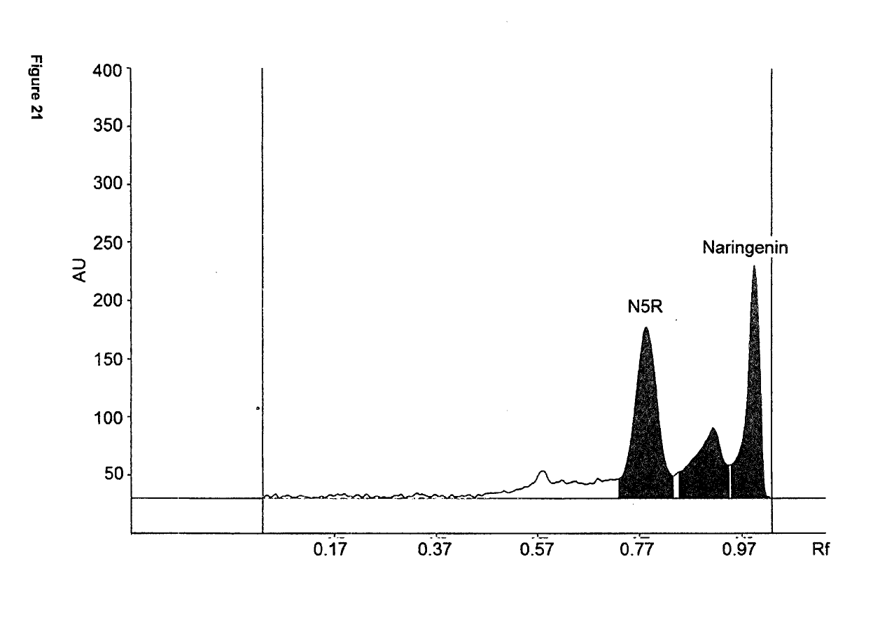

FIG. 22: Cytotoxicity of flavonoid-5-O-α-L-rhamnosides on normal human epidermal keratinocytes

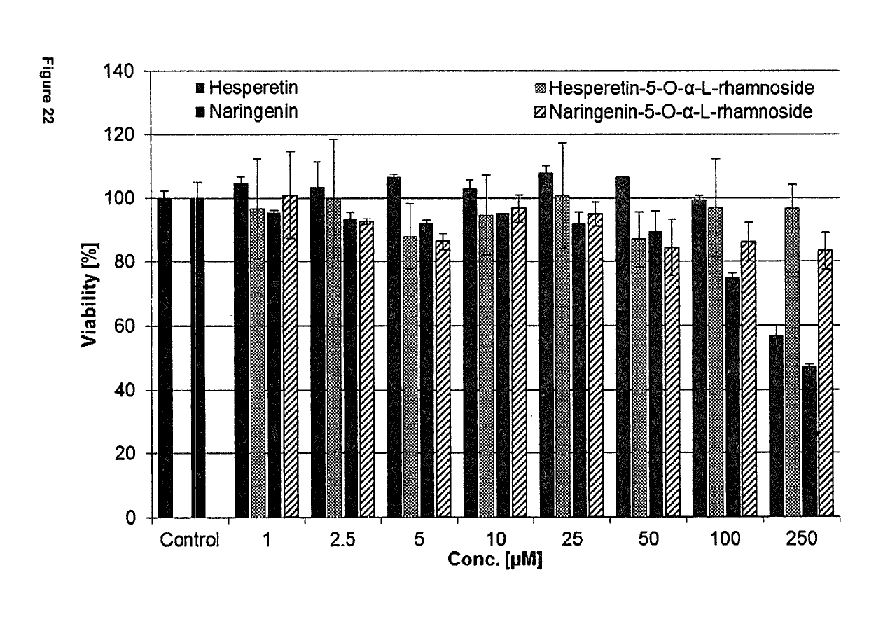

FIG. 23: Antiinflammatory and anti-oxidative (both on normal human epidermal keratinocytes), and synthesis/release stimulating (on normal human dermal fibroblasts or normal human epidermal melanocytes) activities of flavonoid-5-O-α-L-rhamnosides; Activities are given in percent in relation to control experiments

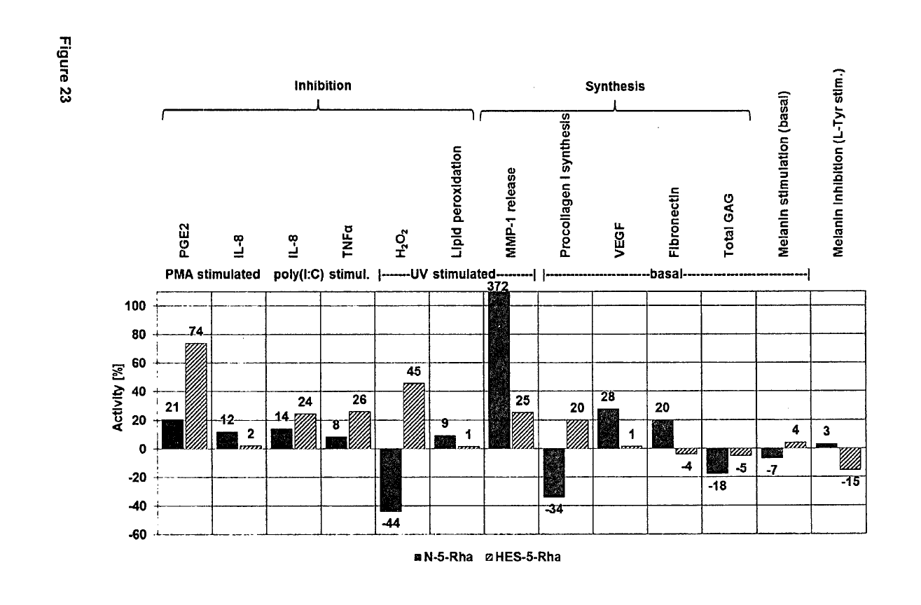

### DETAILED DESCRIPTION OF THE INVENTION

The present invention provides a compound of the following formula (I)

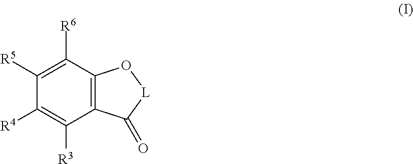

The present invention also provides compositions comprising a compound of formula (I) or a pharmaceutically, cosmetically or nutritionally acceptable salt, solvate or prodrug thereof, in combination with a pharmaceutically, cosmetically or nutritionally acceptable excipient.

The invention furthermore relates to the use of a compound of formula (I) or a pharmaceutically acceptable salt, solvate or prodrug thereof in the preparation of a medicament for the treatment or prevention of a skin disease, an allergy, an autoimmune disease, a cardiovascular disease, a lung disease, asthma, a bacterial, viral or parasitic disease, metabolic syndrome, cancer, Alzheimer's disease, arthritis, dysfunctional hair growth, dysfunctional wound healing, or diabetes.

The invention likewise provides a method of treating or preventing a skin disease, an allergy, an autoimmune disease, a cardiovascular disease, a lung disease, asthma, a bacterial, viral or parasitic disease, metabolic syndrome, cancer, Alzheimer's disease or diabetes, the method comprising administering a compound of formula (I) or a pharmaceutically acceptable salt, solvate or prodrug thereof, or a pharmaceutical composition comprising any of the aforementioned entities and a pharmaceutically acceptable excipient, to a subject (e.g., a human) in need thereof.

The compounds of formula (I) will be described in more detail in the following:

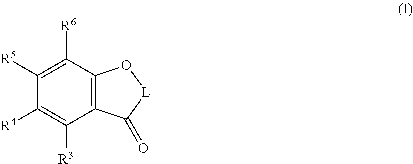

Compounds of formula (I) comprise compounds of formulae (II), (IIa), (IIb), (IIc), (IId), (III) and (IV). Any reference to a compound of formula (I) or compounds of formula (I) is therefore to be understood as also referring to any one of compounds of formulae (II), (IIa), (IIb), (IIc), (IId), (III) and (IV) and to the more specific examples thereof which are disclosed herein.

### Definitions

As used herein, the term “flavonoid-type compound” refers to any compounds falling under the general formula (I) and is thus not limited to compounds which are generally considered flavonoid-type compounds.

As used herein, the term “hydrocarbon group” refers to a group consisting of carbon atoms and hydrogen atoms. Examples of this group are alkyl, alkenyl, alkynyl, alkylene, carbocyl and aryl. Both monovalent and divalent groups are encompassed.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more carbon-to-carbon double bonds. The term “C₂₋₅ alkynyl” denotes an alkynyl group having 2 to 5 carbon atoms. Preferred exemplary alkynyl groups are ethynyl, propynyl, or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “Co alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. Unless defined otherwise, “heterocyclyl” preferably refers to heteroaryl, heterocycloalkyl or heterocycloalkenyl.

As used herein, the term “heterocyclic ring” refers to saturated or unsaturated rings containing one or more heteroatoms, preferably selected from oxygen, nitrogen and sulfur. Examples include heteroaryl and heterocycloalkyl as defined herein. Preferred examples contain, 5 or 6 atoms, particular examples, are 1,4-dioxane, pyrrole and pyridine.

The term “carbocyclic ring” means saturated or unsaturated carbon rings such as aryl or cycloalkyl, preferably containing 5 or 6 carbon atoms. Examples include aryl and cycloalkyl as defined herein.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), anthracenyl, or phenanthrenyl. Unless defined otherwise, an “aryl” preferably has 6 to 14 ring atoms, more preferably 6 to 10 ring atoms, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromenyl, xanthenyl, phenoxathiinyl, pyrrolyl (e.g., 2H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, pteridinyl, carbazolyl, beta-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, furazanyl, phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzoxazolyl, benzisoxazolyl, benzimidazolyl, 1H-tetrazolyl, 2H-tetrazolyl, coumarinyl, or chromonyl. Unless defined otherwise, a “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

The term “heteroalkyl” refers to saturated linear or branched-chain monovalent hydrocarbon radical of one to twelve carbon atoms, including from one to six carbon atoms and from one to four carbon atoms, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkyl radical may be optionally substituted independently with one or more substituents described herein. The term “heteroalkyl” encompasses alkoxy and heteroalkoxy radicals.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heterocycloalkyl” may, e.g., refer to oxetanyl, tetrahydrofuranyl, piperidinyl, piperazinyl, aziridinyl, azetidinyl, pyrrolidinyl, imidazolidinyl, morpholinyl (e.g., morpholin-4-yl), pyrazolidinyl, tetrahydrothienyl, octahydroquinolinyl, octahydroisoquinolinyl, oxazolidinyl, isoxazolidinyl, azepanyl, diazepanyl, oxazepanyl or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂.

As used herein, the term “rhamnosyl” refers to a substituted or unsubstituted rhamnose residue which is preferably connected via the C1-OH group of the same.

The term “monosaccharide” as used herein refers to sugars which consist of only a single sugar unit. These include all compounds which are commonly referred to as sugars and includes sugar alcohols and amino sugars. Examples include tetroses, pentoses, hexoses and heptoses, in particular aldotetroses, aldopentoses, aldohexoses and aldoheptoses.

Aldotetroses include erythrose and threose and the ketotetroses include erythrulose.

Aldopentoses include apiose, ribose, arabinose, lyxose, and xylose and the ketopentoses include ribulose and xylulose. The sugar alcohols which originate in pentoses are called pentitols and include arabitol, xylitol, and adonitol. The saccharic acids include xylosaccharic acid, ribosaccharic acid, and arabosaccharic acid.

Aldohexoses include galactose, talose, altrose, allose, glucose, idose, mannose, rhamnose, fucose, olivose, rhodinose, and gulose and the ketohexoses include tagatose, psicose, sorbose, and fructose. The hexitols which are sugar alcohols of hexose include talitol, sorbitol, mannitol, iditol, allodulcitol, and dulcitol. The saccharic acids of hexose include mannosaccharic acid, glucosaccharic acid, idosaccharic acid, talomucic acid, alomucic acid, and mucic acid.

Examples of aldoheptoses are idoheptose, galactoheptose, mannoheptose, glucoheptose, and taloheptose. The ketoheptoses include alloheptulose, mannoheptulose, sedoheptulose, and taloheptulose.

Examples of amino sugars are fucosamine, galactosamine, glucosamine, sialic acid, N-acetylglucosamine, and N-acetylgalactosamine.

As used herein, the term “disaccharide” refers to a group which consists of two monosaccharide units. Disaccharides may be formed by reacting two monosaccharides in a condensation reaction which involves the elimination of a small molecule, such as water.

Examples of disaccharides are maltose, isomaltose, lactose, nigerose, sambubiose, sophorose, trehalose, saccharose, rutinose, and neohesperidose.

As used herein, the term “oligosaccharide” refers to a group which consists of three to eight monosaccharide units. Oligosaccharide may be formed by reacting three to eight monosaccharides in a condensation reaction which involves the elimination of a small molecule, such as water. The oligosaccharides may be linear or branched.

Examples are dextrins as maltotriose, maltotetraose, maltopentaose, maltohexaose, maltoheptaose, and maltooctaose, fructo-oligosaccharides as kestose, nystose, fructosylnystose, bifurcose, inulobiose, inulotriose, and inulotetraose, galacto-oligosaccharides, or mannan-oligosaccharides.

As used herein, the expression “the compound contains at least one OH group in addition to any OH groups in R³” indicates that there is at least one OH group in the compound at a position other than residue R³. Examples of the OH groups in R³ are OH groups of the rhamnosyl group or of any substituents thereof. Consequently, for the purpose of determining whether the above expression is fulfilled, the residue R³ is disregarded and the number of the remaining OH groups in the compound is determined.

As used herein, the expression “an OH group directly linked to a carbon atom being linked to a neighboring carbon or nitrogen atom via a double bond” indicates a group of the following partial structure:

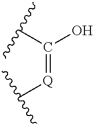

in which Q is N or C which may be further substituted. The double bond between C and Q may be part of a larger aromatic system and may thus be delocalized. Examples of such OH groups include OH groups which are directly attached to aromatic moieties, such as, aryl or heteroaryl groups. One specific example is a phenolic OH group.

As used herein, the term “substituted at one or more of its —OH groups” indicates that a substituent may be attached to one or more of the “—OH” groups in such a manner that the resulting group may be represented by “—O-substituent”.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

When specific positions in the compounds of formula (I) or formula (II) are referred to, the positions are designated as follows:

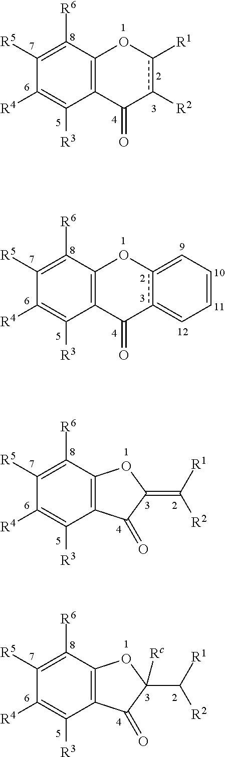

A skilled person will appreciate that the substituent groups comprised in the compounds of formula (I) may be attached to the remainder of the respective compound via a number of different positions of the corresponding specific substituent group. Unless defined otherwise, the preferred attachment positions for the various specific substituent groups are as illustrated in the examples.

As used herein, the term “about” preferably refers to ±10% of the indicated numerical value, more preferably to ±5% of the indicated numerical value, and in particular to the exact numerical value indicated.

Compounds Having the General Formula (I)

The present invention relates to a compound of the following formula (I) or a solvate thereof

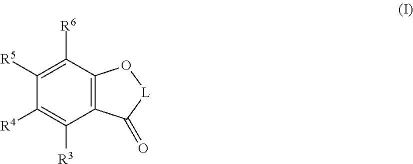

Many examples of the compound of following formula (I) are disclosed herein, such as, compounds of formulae (II), (IIa), (IIb), (IIc), (IId), (III) and (IV). It is to be understood that, if reference is made to the compound of formula (I), this reference also includes any of the compounds of formulae (II), (IIa), (IIb), (IIc), (IId), (III), (IV) etc.

In the present invention, the sign [symbol] represents a double bond or a single bond. In some examples, the sign [symbol] represents a single bond. In other examples, the sign [symbol] represents a double bond.

L is

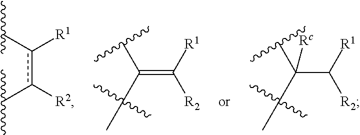

It is preferred that L be

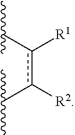

In preferred compounds of formula (I), R¹ and R² are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from —OH.

In preferred compounds of formula (I), R¹ is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}. In more preferred compounds of formula (I), R¹ is selected from cycloalkyl, heterocycloalkyl, aryl and heteroaryl; wherein said cycloalkyl, said heterocycloalkyl, said aryl and said heteroaryl are each optionally substituted with one or more groups R^{c}. In even more preferred compounds of formula (I), R¹ is selected from aryl and heteroaryl; wherein said aryl and said heteroaryl are each optionally substituted with one or more groups R^{c}. In still more preferred compounds of formula (I), R¹ is selected from aryl and heteroaryl; wherein said aryl and said heteroaryl are each optionally substituted with one or more groups R^{c}. In still more preferred compounds of formula (I), R¹ is aryl which is optionally substituted with one or more groups R^{c}. In one compound of formula (I), R¹ is aryl which is optionally substituted with one, two or three groups independently selected from —OH, —O—R^{d} and —O—C₁₋₄ alkyl. Still more preferably, R¹ is phenyl, optionally substituted with one, two or three groups independently selected from —OH, —O—R^{d} and —O—C₁₋₄ alkyl.

In other preferred compounds of formula (I), R² is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}, and wherein R² is different from —OH. In more preferred compounds of formula (I), R² is selected from cycloalkyl, heterocycloalkyl, aryl and heteroaryl; wherein said cycloalkyl, said heterocycloalkyl, said aryl and said heteroaryl are each optionally substituted with one or more groups R^{c}. In even more preferred compounds of formula (I), R² is selected from aryl and heteroaryl; wherein said aryl and said heteroaryl are each optionally substituted with one or more groups R^{c}. In still more preferred compounds of formula (I), R² is selected from aryl and heteroaryl; wherein said aryl and said heteroaryl are each optionally substituted with one or more groups R^{c}. Still more preferably, R² is aryl which is optionally substituted with one or more groups R^{c}. In some compounds of formula (I), R² is aryl which is optionally substituted with one, two or three groups independently selected from —OH, —O—R^{d} and —O—C₁₋₄ alkyl. Still more preferably, R² is phenyl, optionally substituted with one, two or three groups independently selected from —OH, —O—R^{d} and —O—C₁₋₄ alkyl.

Alternatively, R¹ and R² are joined together to form, together with the carbon atom(s) that they are attached to, a carbocyclic or heterocyclic ring being optionally substituted with one or more substituents R^{e}; wherein each R^{e} is independently selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.

Preferably, each R^{e} is independently selected from C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, each R^{e} is independently selected from C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, each R^{e} is independently selected from C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, each R^{e} is independently selected from C₁₋₅ alkyl, C₂₋₅ alkenyl, heteroalkyl, heterocycloalkyl, —OR^{b} and —OR^{d}; wherein said alkyl, said alkenyl, said heteroalkyl and said heterocycloalkyl are each optionally substituted with one or more groups independently selected from halogen, —CF₃, —CN—OH and —O—R^{d}. Still more preferably, each R^{e} is independently selected from —OH, —O—C₁₋₅ alkyl, C₁₋₅ alkyl, C₂₋₅ alkenyl, heteroalkyl, heterocycloalkyl and —OR^{d}; wherein said alkyl, said alkenyl, said heteroalkyl, said heterocycloalkyl and the alkyl in said —O—C₁₋₅ alkyl are each optionally substituted with one or more groups independently selected from halogen, —CF₃, —CN—OH and —O—R^{d}. Still more preferably, each R^{e} is independently selected from —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN—OH and —O—R^{d}. Most preferably, each R^{e} is independently selected from —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d}.

R⁴, R⁵ and R⁶ can independently be selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.

Alternatively, R⁴ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and R⁵ and R⁶ are joined together to form, together with the carbon atoms that they are attached to, a carbocyclic or heterocyclic ring being optionally substituted with one or more substituents R^{c}.

In a further alternative, R⁴ and R⁵ are joined together to form, together with the carbon atoms that they are attached to, a carbocyclic or heterocyclic ring being optionally substituted with one or more substituents R^{c}; and R⁶ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.

R⁴ is preferably selected from hydrogen, C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, R⁴ is selected from hydrogen, C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, R⁴ is selected from hydrogen, C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, R⁴ is selected from hydrogen, C₁₋₅ alkyl, C₂₋₅ alkenyl, heteroalkyl, heterocycloalkyl, —OR^{b} and —OR^{d}; wherein said alkyl, said alkenyl, said heteroalkyl and said heterocycloalkyl are each optionally substituted with one or more groups independently selected from halogen, —CF₃, —CN—OH and —O—R^{d}. Still more preferably, R⁴ is selected from hydrogen, —OH, —O—C₁₋₅ alkyl, C₁₋₅ alkyl, C₂₋₅ alkenyl, heteroalkyl, heterocycloalkyl and —OR^{d}; wherein said alkyl, said alkenyl, said heteroalkyl, said heterocycloalkyl and the alkyl in said —O—C₁₋₅ alkyl are each optionally substituted with one or more groups independently selected from halogen, —CF₃, —CN—OH and —O—R^{d}. Still more preferably, R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN—OH and —O—R^{d}. Most preferably, R⁴ is selected from hydrogen, —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d}.

R⁵ is preferably selected from hydrogen, C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, R⁵ is selected from hydrogen, C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, R⁵ is selected from hydrogen, C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, R⁵ is selected from hydrogen, C₁₋₅ alkyl, C₂₋₅ alkenyl, —R^{a}—OR^{b} and —R^{a}—OR^{d}; wherein said alkyl and said alkenyl are each optionally substituted with one or more groups R^{c}. Still more preferably, R⁵ is selected from hydrogen, C₁₋₅ alkyl, C₂₋₅ alkenyl, —OR^{b} and —OR^{d}; wherein said alkyl and said alkenyl are each optionally substituted with one or more groups R^{c}. Still more preferably, R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c}; Most preferably, R⁵ is selected from hydrogen, —OH, —O—R^{d}, —O-alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁶ is preferably selected from hydrogen, C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, R⁶ is selected from hydrogen, C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, R⁶ is selected from hydrogen, C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, R⁶ is selected from hydrogen, —OH, C₁₋₅ alkyl, C₂₋₅ alkenyl, heterocycloalkyl and —R^{a}—OR^{d}; wherein said alkyl, said alkenyl and said heterocycloalkyl are each optionally substituted with one or more groups R^{c}. Still more preferably, R⁶ is selected from hydrogen, —OH, C₁₋₅ alkyl, C₂₋₅ alkenyl and —R^{a}—OR^{d}; wherein said alkyl and said alkenyl and said heterocycloalkyl are each optionally substituted with one or more groups R^{c}. Still more preferably, R⁶ is selected from hydrogen, —OH, —O—R^{d}, C₁₋₅ alkyl and C₂₋₅ alkenyl, wherein said alkyl and said alkenyl are each optionally substituted with one or more groups R^{c}. Still more preferably, R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —CF₃, —CN—OH and —O—R^{d}. Most preferably, R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —OH and —O—R^{d};

In all compounds of the present invention, each R³ is —O-(rhamnosyl) wherein said rhamnosyl is optionally substituted at one or more of its —OH groups with one or more groups independently selected from C₁₋₅ alkyl, C₂₋₅ alkenyl, C₂₋₅ alkynyl, a monosaccharide, a disaccharide and an oligosaccharide. The rhamnosyl group in —O—R³ may be attached to the —O— group via any position. Preferably, the rhamnosyl group is attached to the —O— group via position C1. The optional substituents may be attached to the rhamnosyl group at any of the remaining hydroxyl groups. In preferred compounds of the present invention, R³ is —O-α-L-rhamnopyranosyl, —O-α-D-rhamnopyranosyl, —O-β-L-rhamnopyranosyl or —O-β-D-rhamnopyranosyl.

In all compounds of the present invention, each R^{a} is independently selected from a single bond, C₁₋₅ alkylene, C₂₋₅ alkenylene, arylene and heteroarylene; wherein said alkylene, said alkenylene, said arylene and said heteroarylene are each optionally substituted with one or more groups R^{c}. Preferably, each R^{a} is independently selected from a single bond, C₁₋₅ alkylene and C₂₋₅ alkenylene; wherein said alkylene and said alkenylene are each optionally substituted with one or more groups R^{c}. More preferably, each R^{a} is independently selected from a single bond, C₁₋₅ alkylene and C₂₋₅ alkenylene; wherein said alkylene and said alkenylene are each optionally substituted with one or more groups independently selected from halogen, —CF₃, —CN, —OH and —O—C₁₋₄ alkyl. Even more preferably, each R^{a} is independently selected from a single bond, alkylene and C₂₋₅ alkenylene; wherein said alkylene and said alkenylene are each optionally substituted with one or more groups independently selected from —OH and —O—C₁₋₄ alkyl. Still more preferably, each R^{a} is independently selected from a single bond and C₁₋₅ alkylene; wherein said alkylene is optionally substituted with one or more groups independently selected from —OH and —O—C₁₋₄ alkyl. Most preferably, each R^{a} is independently selected from a single bond and C₁₋₅ alkylene.

In all compounds of the present invention, each R^{b} is independently selected from hydrogen, C₁₋₅ alkyl, C₂₋₅ alkenyl, C₂₋₅ alkynyl, heteroalkyl, cycloalkyl, heterocycloalkyl, aryl and heteroaryl; wherein said alkyl, said alkenyl, said alkynyl, said heteroalkyl, said cycloalkyl, said heterocycloalkyl, said aryl and said heteroaryl are each optionally substituted with one or more groups R^{c}. Preferably, each R^{b} is independently selected from hydrogen, C₁₋₅ alkyl, C₂₋₅ alkenyl, cycloalkyl, heterocycloalkyl, aryl and heteroaryl; wherein said alkyl, said alkenyl, said cycloalkyl, said heterocycloalkyl, said aryl and said heteroaryl are each optionally substituted with one or more groups R^{c}. More preferably, each R^{b} is independently selected from hydrogen, C₁₋₅ alkyl, C₂₋₅ alkenyl, heterocycloalkyl, aryl and heteroaryl; wherein said alkyl, said alkenyl, said heterocycloalkyl, said aryl and said heteroaryl are each optionally substituted with one or more groups R^{c}. Even more preferably, each R^{b} is independently selected from hydrogen, C₁₋₅ alkyl, C₂₋₅ alkenyl, heterocycloalkyl, aryl and heteroaryl; wherein said alkyl, said alkenyl, said heterocycloalkyl, said aryl and said heteroaryl are each optionally substituted with one or more groups R^{c}. Still more preferably, each R^{b} is independently selected from hydrogen, C₁₋₅ alkyl, C₂₋₅ alkenyl, heterocycloalkyl, aryl and heteroaryl; wherein said alkyl, said alkenyl, said heterocycloalkyl, said aryl and said heteroaryl are each optionally substituted with one or more groups independently selected from halogen, —CF₃, —CN, —OH and —O—C₁₋₄ alkyl. Still more preferably, each R^{b} is independently selected from hydrogen, C₁₋₅ alkyl, C₂₋₅ alkenyl and aryl; wherein said alkyl, said alkenyl and said aryl are each optionally substituted with one or more groups independently selected from halogen, —CF₃, —CN, —OH and —O—C₁₋₄ alkyl. Still more preferably, each R^{b} is independently selected from hydrogen, C₁₋₅ alkyl and aryl; wherein said alkyl and said aryl are each optionally substituted with one or more groups independently selected from halogen, —CF₃, —CN, —OH and —O—C₁₋₄ alkyl. Still more preferably, each R^{b} is independently selected from hydrogen and C₁₋₅ alkyl; wherein said alkyl is optionally substituted with one or more groups independently selected from halogen, —CF₃, —CN, —OH and —O—C₁₋₄ alkyl. Most preferably, each R^{b} is independently selected from hydrogen and C₁₋₅ alkyl; wherein said alkyl is optionally substituted with one or more groups independently selected from halogen.

In all compounds of the present invention, each R^{c} is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.

Preferably, each R^{c} is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl) and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.

More preferably, each R^{c} is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(alkylene)-O(C₁₋₅ alkyl); wherein said alkyl, said alkenyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.

Even more preferably, each R^{c} is independently selected from C₁₋₅ alkyl, C₂₋₅ alkenyl, —(C₀₋₃ alkylene)-OH and —(C₀₋₃ alkylene)-O—R^{d}; wherein said alkyl, said alkenyl and the alkyl or alkylene moieties comprised in any of the aforementioned groups R^{c} are each optionally substituted with one or more groups independently selected from halogen, —CF₃, —CN, —OH, —O—R^{d} and —O—C₁₋₄ alkyl.

Still more preferably, each R^{c} is independently selected from C₁₋₅ alkyl and C₂₋₅ alkenyl; wherein said alkyl and said alkenyl are each optionally substituted with one or more groups independently selected from halogen, —CF₃, —CN, —OH, —O—R^{d} and —O—C₁₋₄ alkyl.

Still more preferably, each R^{c} is independently selected from C₁₋₅ alkyl and C₂₋₅ alkenyl; wherein said alkyl and said alkenyl are each optionally substituted with one or more groups independently selected from halogen.

In all compounds of the present invention, each R^{d} is independently selected from a monosaccharide, a disaccharide and an oligosaccharide.

R^{d} may, e.g., be independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

Specific examples of R^{d} include disaccharides such as maltoside, isomaltoside, lactoside, melibioside, nigeroside, rutinoside, neohesperidoside glucose(1→3)rhamnoside, glucose(1→4)rhamnoside, and galactose(1→2)rhamnoside.

Specific examples of R^{d} further include oligosaccharides as maltodextrins (maltotrioside, maltotetraoside, maltopentaoside, maltohexaoside, maltoseptaoside, maltooctaoside), galacto-oligosaccharides, and fructo-oligosaccharides.

In some of the compound of the present invention, each R^{d} is independently selected from arabinosidyl, galactosidyl, galacturonidyl, mannosidyl, glucosidyl, rhamnosidyl, apiosidyl, allosidyl, glucuronidyl, N-acetyl-glucosaminyl, N-acetyl-mannosaminyl, fucosidyl, fucosaminyl, 6-deoxytalosidyl, olivosidyl, rhodinosidyl, and xylosidyl.

The compound of formula (I) may contain at least one OH group in addition to any OH groups in R³, preferably an OH group directly linked to a carbon atom being linked to a neighboring carbon or nitrogen atom via a double bond. Examples of such OH groups include OH groups which are directly attached to aromatic moieties, such as, aryl or heteroaryl groups. One specific example is a phenolic OH group.

Procedures for introducing additional monosaccharides, disaccharides or oligosacharides at R³, in addition to the rhamnosyl residue, are known in the literature. Examples therefore include the use of cyclodextrin-glucanotranferases (CGTs) and glucansucrases (such as described in EP 1867729 A1) for transfer of glucoside residues at positions C4″-OH and C3″-OH (Shimoda and Hamada 2010, Nutrients 2:171-180, doi: 10.3390/nu2020171, Park 2006, Biosci Biotechnol Biochem, 70(4):940-948, Akiyama et al. 2000, Biosci Biotechnol Biochem 64(10): 2246-2249, Kim et al. 2012, Enzyme Microb Technol 50:50-56).

Furthermore, procedures for attaching secondary glycosylations at C4″ (EP0420376B1, Akiyama et al. 2000, J Food Hyg Soc Japan 41(1):54-60) and for galactosylation of rhamnosides at position C2‘ ’—OH by β-galactosidases are known (Shimizu et al 2006, Biosci Biotechnol Biochem, 70(4):940-948).

GT1s, such as from Bacillus spp., have been reported as being suitable for generating di- or triglucosides (Jung et al. 2010, J Microbiol Biotechnol 20(10):1393-1396, Pandey et al. 2013, Appl Environ Microbiol 79(11):3516, doi 10.1128/AEM.00409-13).

It is also possible to conduct a simultaneous expression of two or more GTs in E. coli. This has been shown for GT1s from Arabidopsis thaliana in the case of rhamnosylations and glucosylations (Kim et al. 2013, Appl Microbiol Biotechnol 97:5275-5282, DOI 10.1007/s00253-013-4844-7). It is thereby possible to generate allosides, glucuronides, N-Ac-glucosamines, fucosides, fucosamines, 6-deoxytalosides, xylosides, olivosides, rhodinosides, and arabinosides (Simkhada et al. 2010, Biotechnol Bioeng 107(1):154-162) DOI 10.1002/bit.22782, Pandey et al. 2013, Appl Microbiol Biotechnol 97:1889-1901, DOI 10.1007/s00253-012-4438-9, Kim et al. 2012, Appl Microbiol Biotechnol 93:2447-2453, DOI 10.1007/s00253-011-3747-8, Yoon et al. 2012, Appl Environ Microbiol 78(12):4256-4262, DOI: 10.1128/AEM.00275-12, Simkhada et al. 2009, Mol Cells 28:397-401, DOI/10.1007/s10059-009-0135-7, Luzhetskyy et al. 2005, ChemBioChem 6:1406-1410, Krauth et al. 2009, Chem Biol 16:28-35, Erb et al. 2009, Appl Microbiol Biotechnol 83:1067-1076, Chang et al. 2011, PNAS 108(43):17649-17654, Yonekura et al. 2008, Plant Cell 20:2160-2176).

Other procedures such as complementary procedures with glycoside-hydrolases (GHs) such as sucrases (EP 1867729 A1), CGTs (EP 2128265 A1, Akiyama et al. 2000, Biosci Biotechnol Biochem, 64(10):2246-2249) and other α-amylases may be considered (WO 2001073106 A1).

The procedures exemplified with respect to the introduction of additional monosaccharides, disaccharides or oligosaccharides may also be employed to introduce the monosaccharides, disaccharides or oligosaccharides in residue R^{d}.

Compounds of Formula (II)

A first example of the compound of formula (I) is a compound of formula (II) or a solvate thereof:

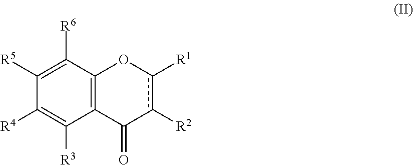

Many examples of the compound of following formula (II) are disclosed herein, such as, compounds of formulae (IIa), (IIb), (IIc) and (IId). It is to be understood that, if reference is made to the compound of formula (II), this reference also includes any of the compounds of formulae (IIa), (IIb), (IIc), (IId), etc.

In formula (II), R¹, R², R³, R⁴, R⁵ and R⁶ are as defined with respect to the compound of general formula (I) including the preferred definitions of each of these residues.

In a first proviso concerning the compound of any of the formulae described herein, and in particular in the compound of formula (II), the compounds naringenin-5-O-α-L-rhamnopyranoside, genistein-5-O-α-L-rhamnopyranoside and eriodictyol-5-O-α-L-rhamnopyranoside are preferably excluded. This proviso is preferably not applicable to any claims relating to the medical use (in particular against arthritis, dysfunctional hair growth and dysfunctional wound healing) or non-medical use of the compounds described herein.

In a second proviso, R¹ in the compound of any of the formulae described herein, and in particular in the compound of formula (II), is preferably not methyl if R⁴ is hydrogen, R⁵ is —OH and [symbol] is a double bond. This proviso is preferably not applicable to any claims relating to the medical use (in particular against arthritis, dysfunctional hair growth and dysfunctional wound healing) or non-medical use of the compounds described herein.

In preferred compounds of formula (II), R¹ is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and R² is selected from hydrogen, C₁₋₅ alkyl and C₂₋₅ alkenyl. In more preferred compounds of formula (II), R¹ is selected from cycloalkyl, heterocycloalkyl, aryl and heteroaryl; wherein said cycloalkyl, said heterocycloalkyl, said aryl and said heteroaryl are each optionally substituted with one or more groups R^{c}; and R² is selected from hydrogen and C₁₋₅ alkyl. In even more preferred compounds of formula (II), R¹ is selected from aryl and heteroaryl; wherein said aryl and said heteroaryl are each optionally substituted with one or more groups R^{c}; and R² is selected from hydrogen and C₁₋₅ alkyl. In still more preferred compounds of formula (II), R¹ is selected from aryl and heteroaryl; wherein said aryl and said heteroaryl are each optionally substituted with one or more groups R^{c}; and R² is selected from hydrogen and C₁₋₅ alkyl. Still more preferably, R¹ is aryl which is optionally substituted with one or more groups R^{c}, and R² is —H. In some compounds of formula (II), R¹ is aryl which is optionally substituted with one, two or three groups independently selected from —OH, —O—R^{d} and —O—C₁₋₄ alkyl, and R² is —H. Still more preferably, R¹ is phenyl, optionally substituted with one, two or three groups independently selected from —OH, —O—R^{d} and —O—C₁₋₄ alkyl; and R² is —H.

In alternatively preferred compounds of formula (II), R² is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from —OH; and R¹ is selected from hydrogen, C₁₋₅ alkyl and C₂₋₅ alkenyl. In more preferred compounds of formula (II), R² is selected from cycloalkyl, heterocycloalkyl, aryl and heteroaryl; wherein said cycloalkyl, said heterocycloalkyl, said aryl and said heteroaryl are each optionally substituted with one or more groups R^{c}; and R¹ is selected from hydrogen and C₁₋₅ alkyl. In even more preferred compounds of formula (II), R² is selected from aryl and heteroaryl; wherein said aryl and said heteroaryl are each optionally substituted with one or more groups R^{c}; and R¹ is selected from hydrogen and C₁₋₅ alkyl. In still more preferred compounds of formula (II), R² is selected from aryl and heteroaryl; wherein said aryl and said heteroaryl are each optionally substituted with one or more groups R^{c}; and R¹ is selected from hydrogen and C₁₋₅ alkyl. Still more preferably, R² is aryl which is optionally substituted with one or more groups R^{c}, and R¹ is —H. In some of the compounds of formula (II), R² is aryl which is optionally substituted with one, two or three groups independently selected from —OH, —O—R^{d} and —O—C₁₋₄ alkyl, and R¹ is —H. Still more preferably, R² is phenyl, optionally substituted with one, two or three groups independently selected from —OH, —O—R^{d} and —O—C₁₋₄ alkyl; and R¹ is —H.

each R^{c} can preferably independently be selected from halogen, —CF₃, —CN, —OH, —O—R^{d}, —O—C₁₋₄ alkyl, —O-aryl, —S—C₁₋₄ alkyl and —S-aryl.

In preferred compounds of formula (II) each R^{d} is independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

The compound of formula (II) may contain at least one OH group in addition to any OH groups in R³, preferably an OH group directly linked to a carbon atom being linked to a neighboring carbon or nitrogen atom via a double bond. Examples of such OH groups include OH groups which are directly attached to aromatic moieties, such as, aryl or heteroaryl groups. One specific example is a phenolic OH group.

R⁴, R⁵ and R⁶ may each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl).

In some compounds of formula (II), R⁵ is —OH, —O—R^{d} or —O—(C₁₋₅ alkyl). In some compounds of formula (II), R⁴ and/or R⁶ is/are hydrogen or —OH. Most preferably, R² is H or —(C₂₋₅ alkenyl).

Furthermore, R¹ and/or R² may independently be selected from aryl and heteroaryl, wherein said aryl and said heteroaryl are each optionally substituted with one or more groups R^{c}.

Compounds of Formula (IIa)

A first example of the compound of formula (II) is a compound of the following formula (IIa) or a solvate thereof:

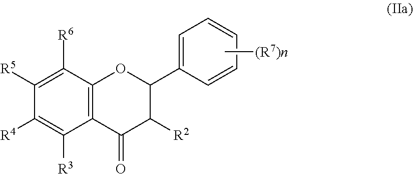

wherein:

R², R³, R⁴, R⁵ and R⁶ are as defined with respect to the compound of general formula (I) including the preferred definitions of each of these residues;

each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl;

n is an integer of 0 to 5, preferably 1, 2, or 3.

Preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl) and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

More preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(alkylene)-O(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

Even more preferably, each R⁷ is independently selected from C₁₋₅ alkyl, C₂₋₅ alkenyl, —(C₀₋₃ alkylene)-OH and —(C₀₋₃ alkylene)-O—R^{d}; wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d} and —O—C₁₋₄ alkyl.

The following combination of residues is preferred in compounds of formula (IIa),

R² is selected from hydrogen, C₁₋₅ alkyl, C₂₋₅ alkenyl, and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};

R⁴ is selected from hydrogen, —OH, —O—R^{d}, C₁₋₅ alkyl, C₂₋₅ alkenyl and —O—C₁₋₅ alkyl; wherein said alkyl, said alkenyl and the alkyl in said —O—C₁₋₅ alkyl are each optionally substituted with one or more groups independently selected from halogen, —CF₃, —CN, —OH and —O—R^{d};

R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};

R⁶ is selected from hydrogen, —OH, —O—R^{d}, C₁₋₅ alkyl and C₂₋₅ alkenyl, wherein said alkyl and said alkenyl are each optionally substituted with one or more groups R^{c};

each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C-s alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl; and

n is an integer of 0 to 3.

The following combination of residues is more preferred in compounds of formula (IIa),

R² is selected from hydrogen, C₁₋₅ alkyl and C₂₋₅ alkenyl, wherein said alkyl and said alkenyl are each optionally substituted with one or more groups independently selected from halogen, —OH and —O—R^{d};

R⁴ is selected from hydrogen, —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d};

R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —OH and —O—R^{d};

each R⁷ is independently selected from C₁₋₅ alkyl, C₂₋₅ alkenyl, —(C₀₋₃ alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH, and —O—R^{d}; and

n is 0, 1 or 2.

Even more preferably, the compound of formula (IIa), is selected from the following compounds or solvates thereof:

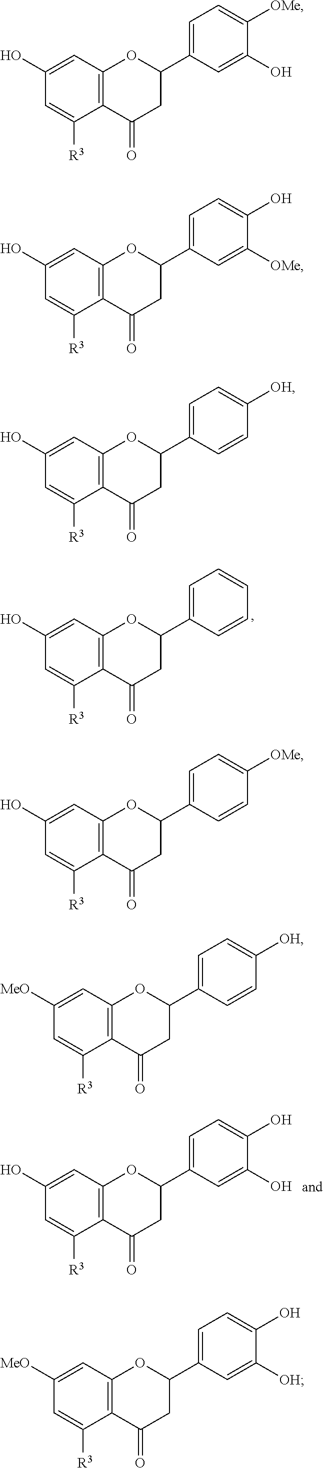

wherein R³ is as defined with respect to the compound of general formula (I).

Compounds of Formula (IIb)

A second example of the compound of formula (II) is a compound of the following formula (IIb) or a solvate thereof:

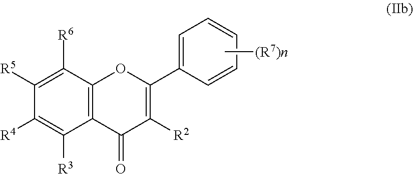

wherein:

R², R³, R⁴, R⁵ and R⁶ are as defined with respect to the compound of general formula (I) including the preferred definitions of each of these residues;

each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl; and

n is an integer of 0 to 5, preferably 1, 2, or 3.

Preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

More preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(alkylene)-O(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

Even more preferably, each R⁷ is independently selected from C₁₋₅ alkyl, C₂₋₅ alkenyl, —(C₀₋₃ alkylene)-OH and —(C₀₋₃ alkylene)-O—R^{d}; wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d} and —O—C₁₋₄ alkyl.

The following combination of residues is preferred in compounds of formula (IIb),

R² is selected from hydrogen, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};

R³ is as defined with respect to the compound of general formula (I);

R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};

R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};

R⁶ is selected from hydrogen, —OH, —O—R^{d}, C₁₋₅ alkyl and C₂₋₅ alkenyl; wherein said alkyl and said alkenyl are each optionally substituted with one or more groups R^{c};

each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C-s alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl; and

n is an integer of 0 to 3.

The following combination of residues is more preferred in compounds of formula (IIb),

R² is selected from hydrogen, C₁₋₅ alkyl and C₂₋₅ alkenyl, wherein said alkyl and said alkenyl are each optionally substituted with one or more groups independently selected from halogen, —OH and —O—R^{d};

R³ is as defined with respect to the compound of general formula (I);

R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁵ is selected from hydrogen, —OH, —O—R^{d}, —O—C₁₋₅ alkyl and C₂₋₅ alkenyl, wherein the alkyl in said —O—C₁₋₅ alkyl and said alkylene are each optionally substituted with one or more groups independently selected from halogen, —OH and —O—R^{d};

R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};

each R⁷ is independently selected from C₁₋₅ alkyl, C₂₋₅ alkenyl, —(C₀₋₃ alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH and —O—R^{d}; and

n is 0, 1 or 2.

Even more preferably, the compound is selected from the following compounds or solvates thereof:

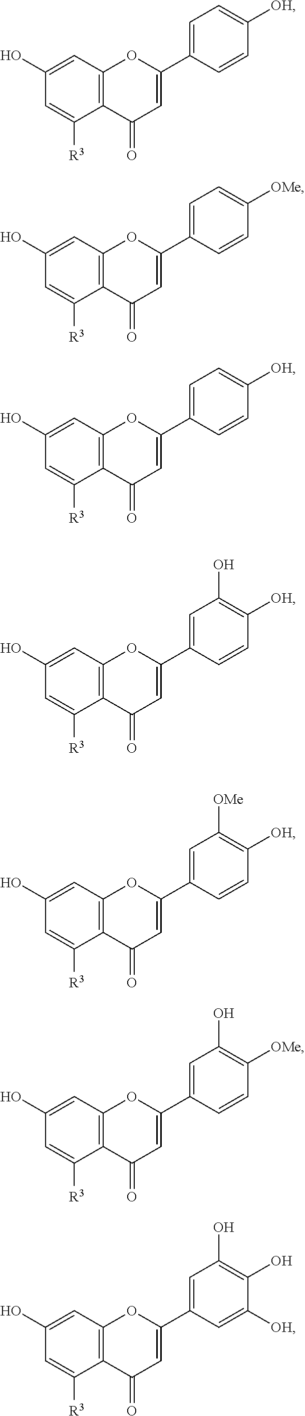

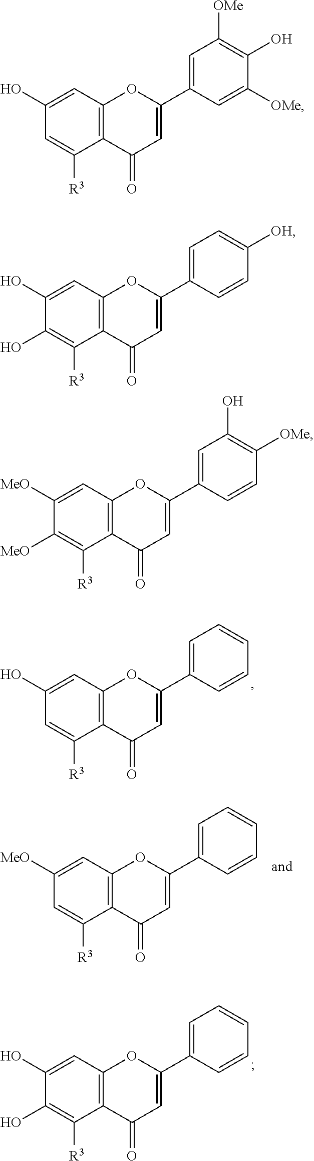

wherein R³ is as defined with respect to the compound of general formula (I).

Compounds of Formula (IIc)

A third example of the compound of formula (II) is a compound of the following formula (IIc) or a solvate thereof:

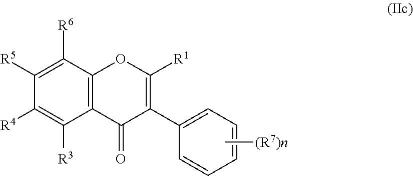

wherein:

R¹, R³, R⁴, R⁵ and R⁶ are as defined with respect to the compound of general formula (I) including the preferred definitions of each of these residues;

each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl; and

n is an integer of 0 to 5, preferably 1, 2, or 3.

Preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

More preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(alkylene)-O(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

Even more preferably, each R⁷ is independently selected from C₁₋₅ alkyl, C₂₋₅ alkenyl, —(C₀₋₃ alkylene)-OH, —(C₀₋₃ alkylene)-O—R^{d}; wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d} and —O—C₁₋₄ alkyl.

The following combination of residues is preferred in compounds of formula (IIc),

R¹ is selected from hydrogen, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};

R³ is as defined with respect to the compound of general formula (I);

R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN—OH and —O—R^{d};

R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};

R⁶ is selected from hydrogen, —OH, —O—R^{d}, C₁₋₅ alkyl and C₂₋₅ alkenyl, wherein said alkyl and said alkenyl are each optionally substituted with one or more groups R^{c};

each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C-s alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl; and

n is an integer of 0 to 3.

The following combination of residues is more preferred in compounds of formula (IIc),

R¹ is selected from hydrogen, C₁₋₅ alkyl and C₂₋₅ alkenyl, wherein said alkyl and said alkenyl are each optionally substituted with one or more groups independently selected from halogen, —OH and —O—R^{d};

R³ is as defined with respect to the compound of general formula (I);

R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};

each R⁷ is independently selected from C₁₋₅ alkyl, C₂₋₅ alkenyl, —(C₀₋₃ alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH and —O—R^{d}; and

n is 0, 1 or 2.

Even more preferred are compounds of formula (IIc), which are is selected from the following compounds or solvates thereof:

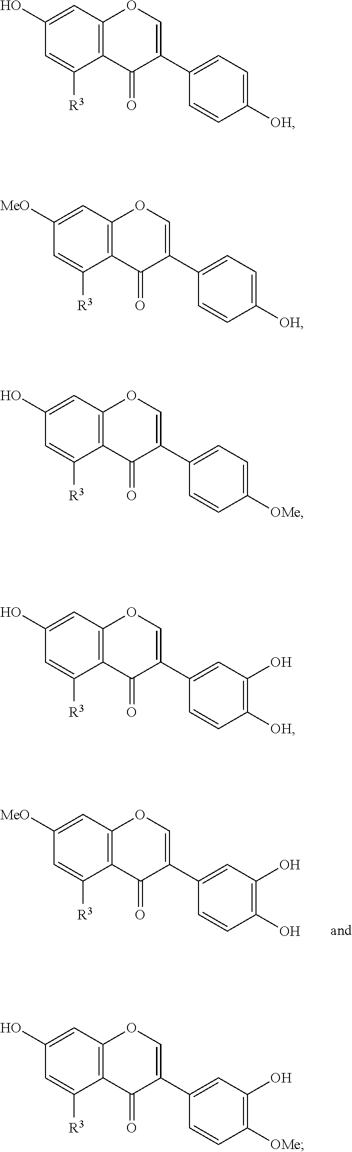

wherein R³ is as defined with respect to the compound of general formula (I).

Compounds of Formula (IId)

A fourth example of the compound of formula (II) is a compound of the following formula (IId) or a solvate thereof:

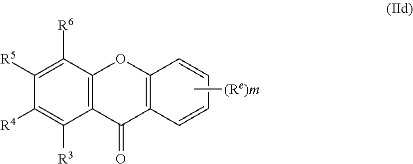

wherein:

R³, R⁴, R⁵, R⁶ and R^{e} are as defined with respect to the compound of general formula (I) including the preferred definitions of each of these residues; and

m is an integer of 0 to 4, preferably 0 to 3, more preferably 1 to 3, even more preferably 1 or 2.

The following combination of residues is preferred in compounds of formula (IId),

R³ is as defined with respect to the compound of general formula (I);

R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN—OH and —O—R^{d};

R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};

R⁶ is selected from hydrogen, —OH, —O—R^{d}, C₁₋₅ alkyl and C₂₋₅ alkenyl, wherein said alkyl and said alkenyl are each optionally substituted with one or more groups R^{c};

each R^{e} is independently selected from —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c}; and

m is an integer of 0 to 3.

The following combination of residues is more preferred in compounds of formula (IId),

R³ is as defined with respect to the compound of general formula (I);

R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};

each R^{e} is independently selected from —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d}; and

m is 0, 1 or 2.

Even more preferred examples of the compound of formula (IId), are compounds selected from the following compounds or solvates thereof:

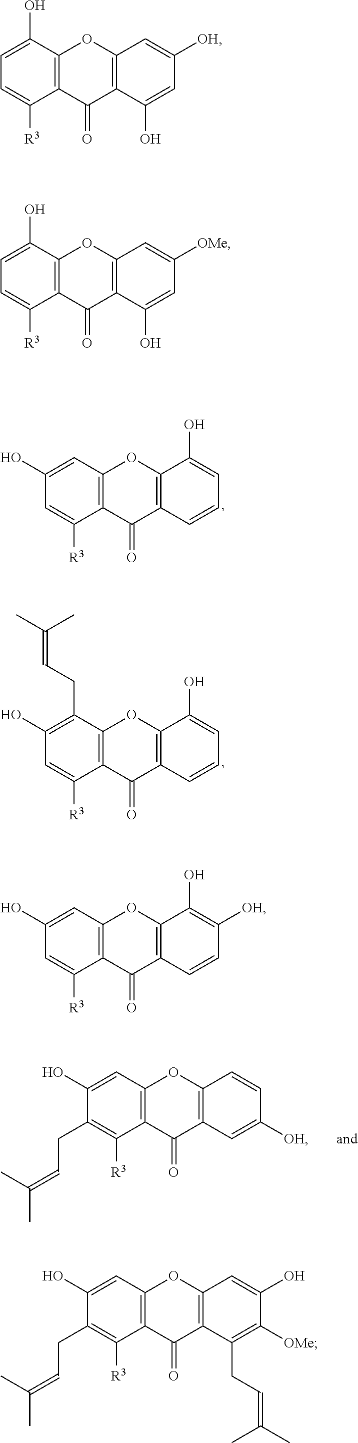

wherein R³ is as defined with respect to the compound of general formula (I).

In preferred compounds of formulae (II), (IIa), (IIb), (IIc) and (IId), R³ is —O-α-L-rhamnopyranosyl, —O-α-D-rhamnopyranosyl, —O-β-L-rhamnopyranosyl or —O-β-D-rhamnopyranosyl.

Compounds of Formula (III)

A second example of a compound of formula (I) is a compound of formula (III) or a solvate thereof:

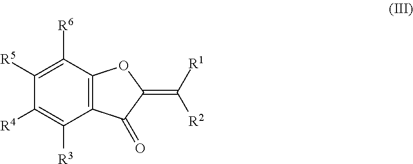

wherein R¹, R², R³, R⁴, R⁵ and R⁶ are as defined with respect to the compound of general formula (I) including the preferred definitions of each of these residues.

In a preferred example of the compounds of formulae (III), R¹ is selected from aryl and heteroaryl, wherein said aryl and said heteroaryl are each optionally substituted with one or more groups R^{c}.

In a preferred example of the compounds of formulae (III), each R^{c} is independently selected from halogen, —CF₃, —CN, —OH, —O—R^{d}, —O—C₁₋₄ alkyl, —O-aryl, —S—C₁₋₄ alkyl and —S-aryl.

In a preferred example of the compounds of formulae (III), the compound contains at least one OH group in addition to any OH groups in R³, preferably an OH group directly linked to a carbon atom being linked to a neighboring carbon or nitrogen atom via a double bond.

In a preferred example of the compounds of formulae (III), R⁴, R⁵ and R⁶ are each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl).

In a preferred example of the compounds of formulae (III), R⁵ is —OH, —O—R^{d} or —O—(C₁₋₅ alkyl).

In a preferred example of the compounds of formulae (III), R⁴ and/or R⁶ is/are hydrogen or —OH.

Particular examples of the compound of formula (III) include the following compounds or solvates thereof:

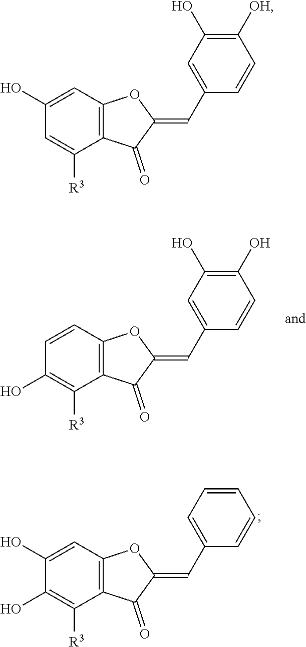

wherein R³ is as defined with respect to the compound of general formula (I).

In a preferred example of the compounds of formula (III), R³ is —O-α-L-rhamnopyranosyl, —O-α-D-rhamnopyranosyl, —O-β-L-rhamnopyranosyl or —O-β-D-rhamnopyranosyl.

In a preferred example of the compounds of formula (III), each R^{d} is independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

Compounds of Formula (IV)

Yet a further example of a compound of formula (I) is a compound of formula (IV) or a solvate thereof:

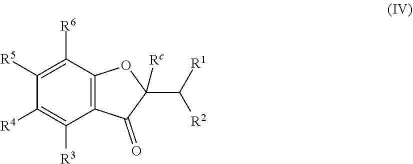

wherein R¹, R², R³, R⁴, R⁵, R⁶ and R^{c} are as defined with respect to the compound of general formula (I) including the preferred definitions of each of these residues.

In a preferred example of the compounds of formula (IV), R¹ is selected from aryl and heteroaryl, wherein said aryl and said heteroaryl are each optionally substituted with one or more groups R^{c}.

In a preferred example of the compounds of formula (IV), each R^{c} is independently selected from halogen, —CF₃, —CN, —OH, —O—R^{d}, —O—C₁₋₄ alkyl, —O-aryl, —S—C₁₋₄ alkyl and —S-aryl.

In a preferred example of the compounds of formula (IV), the compound contains at least one OH group in addition to any OH groups in R³, preferably an OH group directly linked to a carbon atom being linked to a neighboring carbon or nitrogen atom via a double bond.

In a preferred example of the compounds of formula (IV), R⁴, R⁵ and R⁶ are each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl).

In a preferred example of the compounds of formula (IV), R⁵ is —OH, —O—R^{d} or —O—(C₁₋₅ alkyl).

In a preferred example of the compounds of formula (IV), R⁴ and/or R⁶ is/are hydrogen or —OH.

Particular examples of the compound of formula (IV) include the following compounds or solvates thereof:

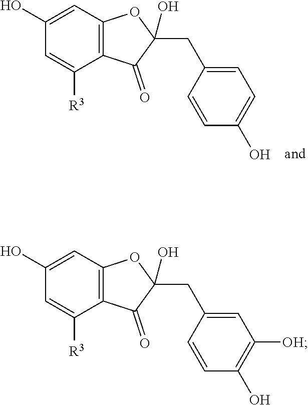

wherein R³ is as defined with respect to the compound of general formula (I).

In a preferred example of the compounds of formula (IV), R³ is —O-α-L-rhamnopyranosyl, —O-α-D-rhamnopyranosyl, —O-β-L-rhamnopyranosyl or —O-β-D-rhamnopyranosyl.

In a preferred example of the compounds of formula (IV), each R^{d} is independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

Pharmaceutical Use of the Compounds of the Present Invention

The present invention further relates to a pharmaceutical composition comprising the compounds of formulae (I), (II), (IIa), (IIb), (IIc), (IId), (III) and (IV) and optionally a pharmaceutically acceptable excipient.

The compounds and the pharmaceutical composition of the present invention are particularly suitable for the treatment or prevention of a disease and/or condition selected from a skin disease, an allergy, an autoimmune disease, a cardiovascular disease, a lung disease, asthma, a bacterial, viral or parasitic disease, metabolic syndrome, cancer, Alzheimer's disease, arthritis, dysfunctional hair growth, dysfunctional wound healing, or diabetes, but are not limited thereto. The compounds and the pharmaceutical composition of the present invention are preferably used for the treatment or prevention of a disease and/or condition selected from arthritis, dysfunctional hair growth (preferably referring to any conditions wherein hair growth is diminished), dysfunctional wound healing (preferably referring to any conditions wherein wound healing is diminished). Furthermore, collagen synthesis or fibronectin synthesis may be promoted which supports a firm skin, reduces wrinkles and diminishes skin aging. An example of abnormal collagene syndroms, which may be treated by the compounds and compositions of the present invention, is Dupuytren's contracture.

Alternatively, the disease and/or condition may be selected from a skin disease, an allergy, an autoimmune disease, a cardiovascular disease, a lung disease, asthma, a bacterial, viral or parasitic disease, metabolic syndrome, cancer, Alzheimer's disease, arthritis, dysfunctional hair growth, dysfunctional wound healing, or diabetes, but are not limited thereto.

Skin diseases include all kinds of dermatitis (Kim et al. 2007, Biol Pharm Bull 30:2345-2351, 10.1248/bpb.30.2345, Kempuraj et al. 2008, Br J Pharmacol 155:1076-1084, 10.1038/bjp.2008.356), atopic dermatitis (Ahn et al. 2010, Phytother Res 24:1071-1077, 10.1002/ptr.3084), psoriasis (Weng et al. 2014, PLoS One 9:e90739, 10.1371/journal.pone.0090739) and akne (Sato et al. 2007, J Invest Dermatol 127:2740-2748, 10.1038/sj.jid.5700927).

The use of flavonoid-type compounds as anti-allergics has also been described (Kawai et al. 2007, Allergology International 56:113-123, 10.2332/allergolint.R-06-135).

The treatment of cardiovascular diseases has been reported (Hertog et al. 1993, The Lancet 342:1007-1011, Li et al. 2004, Carbohydr Res 339:2789-2797, Majewska-Wierzbicka and Czeczot 2012, Pol Merkur Lekarski 32:50-54, Prahalathan et al. 2012, Metabolism 61:1087-1099, 10.1016/j.metabol.2011.12.012, Assini et al. 2013, Current Opinion in Lipidology 24:34-40, 10.1097/MOL.0b013e32835c07fd, Testai et al. 2013, Journal of Pharmacy and Pharmacology 65:750-756, 10.1111/jphp.12032).

Furthermore, flavonoid-type compounds have been reported to be active in the treatment of asthma (Shi et al. 2009, Canadian Journal of Physiology & Pharmacology 87:729-735, 10.1139/Y09-065, Tanaka and Takahashi 2013, Nutrients 5:2128-2143, 10.3390/nu5062128, Yang et al. 2013, Phytotherapy Research 27:1381-1391, 10.1002/ptr.4862).

Flavonoid-type compounds have been found to be useful in the treatment of viral infections (Malhotra et al. 1996, Phytochemistry 43:1271-1276, 10.1016/S0031-9422(95)00522-6, Choi et al. 2009, Antiviral Research 81:77-81, 10.1016/j.antiviral.2008.10.002), in particular against influenza (Choi et al. 2009, European Journal of Pharmaceutical Sciences 37:329-333, http://dx.doi.org/10.1016/j.ejps.2009.03.002, Choi et al. 2012, Phytotherapy Research 26:462-464, 10.1002/ptr.3529), hepatitis (Gao et al. 2009, Carbohydr Res 344:511-515, Goldwasser et al. 2011, Journal of Hepatology 55:963-971, 10.1016/j.jhep.2011.02.011) and HIV (Andrae-Marobela et al. 2013, Curr Drug Metab 14:392-413, 10.2174/13892002113149990095).

A large variety of flavonoid-type compounds have been shown to have activity against cancer (Jin et al. 2013, Oncol Rep 30:2336-2342, 10.3892/or.2013.2711), in particular prostate cancer (Lai et al. 2013, Food Funct 4:944-949, 10.1039/c3fo60037h), melanoma (Lee et al. 2011, J Biol Chem 286:14246-14256, 10.1074/jbc.M110.147348) and liver cancer (Androutsopoulos and Spandidos 2013, Journal of Nutritional Biochemistry 24:496-504, 10.1016/j.jnutbio.2012.01.012).

Further applications of flavonoid-type compounds include the treatment of Alzheimer's disease (Sato et al. 2013, J Biol Chem 288:23212-23224, 10.1074/jbc.M113.464222) and diabetes (Mulvihill et al. 2009, Diabetes 58:2198-2210, 10.2337/db09-0634, Assini, Mulvihill et al. 2013, Current Opinion in Lipidology 24:34-40, 10.1097/MOL.0b013e32835c07fd, Babu et al. 2013, Journal of Nutritional Biochemistry 24:1777-1789, 10.1016/j.jnutbio.2013.06.003)

The scope of the invention embraces all pharmaceutically, cosmetically and nutritionally acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically, cosmetically and nutritionally acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically, cosmetically and nutritionally acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

Moreover, the scope of the invention embraces the compounds of formula (I) in any solvated form, including, e.g., solvates with water, for example hydrates, or with organic solvents such as, e.g., methanol, ethanol or acetonitrile, i.e., as a methanolate, ethanolate or acetonitrilate, respectively, or in the form of any polymorph. It is to be understood that such solvates of the compounds of the formula (I) also include solvates of pharmaceutically, cosmetically and nutritionally acceptable salts of the compounds of the formula (I).

Furthermore, the compounds of formula (I) may exist in the form of different isomers, in particular stereoisomers (including, e.g., geometric isomers (or cis/trans isomers), enantiomers and diastereomers) or tautomers. All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

Pharmaceutically acceptable prodrugs of the compounds of formula (I) are derivatives which have chemically or metabolically cleavable groups and become, by solvolysis or under physiological conditions, the compounds of formula (I) which are pharmaceutically, in vivo. Prodrugs of the compounds according to the the present invention may be formed in a conventional manner with a functional group of the compounds such as, e.g., with an amino, hydroxy or carboxy group. The prodrug form often offers advantages in terms of solubility, tissue compatibility or delayed release in a mammalian organism (see, Bundgaard, H., Design of Prodrugs, pp. 7-9, 21-24, Elsevier, Amsterdam 1985). Prodrugs include acid derivatives, such as, e.g., esters prepared by reaction of the parent acidic compound with a suitable alcohol, or amides prepared by reaction of the parent acid compound with a suitable amine. If a compound of the present invention has a carboxyl group, an ester derivative prepared by reacting the carboxyl group with a suitable alcohol or an amide derivative prepared by reacting the carboxyl group with a suitable amine is exemplified as a prodrug. An especially preferred ester derivative as a prodrug is methylester, ethylester, n-propylester, isopropylester, n-butylester, isobutylester, tert-butylester, morpholinoethylester, N,N-diethylglycolamidoester or α-acetoxyethylester. If a compound of the present invention has a hydroxy group, an acyloxy derivative prepared by reacting the hydroxyl group with a suitable acylhalide or a suitable acid anhydride is exemplified as a prodrug. An especially preferred acyloxy derivative as a prodrug is —OC(═O)—CH₃, —OC(═O)—C₂H₅, —OC(═O)-(tert-Bu), —OC(═O)—C₁₅H₃₁, —OC(═O)-(m-COONa-Ph), —OC(═O)—CH₂CH₂COONa, —O(C═O)—CH(NH₂)CH₃ or —OC(═O)—CH₂—N(CH₃)₂. If a compound of the present invention has an amino group, an amide derivative prepared by reacting the amino group with a suitable acid halide or a suitable mixed anhydride is exemplified as a prodrug. An especially preferred amide derivative as a prodrug is —NHC(═O)—(CH₂)₂OCH₃ or —NHC(═O)—CH(NH₂)CH₃.

The compounds provided herein may be administered as compounds per se or may be formulated as medicaments. The medicaments/pharmaceutical compositions may optionally comprise one or more pharmaceutically, cosmetically or nutritionally acceptable excipients, such as carriers, diluents, fillers, disintegrants, lubricating agents, binders, colorants, pigments, stabilizers, preservatives, antioxidants, and/or solubility enhancers.

In particular, the pharmaceutical compositions may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatine capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, and vaginal.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci. (USA) 77:4030-4034 (1980); EP0052322; EP0036676; EP088046; EP0143949; EP0142641; JP 83-118008; U.S. Pat. Nos. 4,485,045; 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to the emulsification/spray drying process disclosed in WO 99/16419 or WO 01/85136. Spray drying of solution formulations of the compounds of the present invention can be carried out, e.g., as described generally in the “Spray Drying Handbook”, 5th ed., K. Masters, John Wiley & Sons, Inc., NY (1991), and in WO 97/41833 or WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration of the compounds or pharmaceutical compositions of the present invention are oral administration or parenteral administration (e.g., subcutaneous or intravenous administration), and most preferably a compound or a pharmaceutical composition of the invention is to be administered orally.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The subject or patient, such as the subject in need of treatment or prevention, may be an animal (e.g., a non-human animal), a vertebrate animal, a mammal, a rodent (e.g., a guinea pig, a hamster, a rat, a mouse), a murine (e.g., a mouse), a canine (e.g., a dog), a feline (e.g., a cat), a porcine (e.g., a pig), an equine (e.g., a horse), a primate, a simian (e.g., a monkey or ape), a monkey (e.g., a marmoset, a baboon), an ape (e.g., a gorilla, chimpanzee, orang-utan, gibbon), or a human. In the context of this invention, it is particularly envisaged that animals are to be treated which are economically, agronomically or scientifically important. Scientifically important organisms include, but are not limited to, mice, rats, and rabbits. Lower organisms such as, e.g., fruit flies like Drosophila melanogaster and nematodes like Caenorhabditis elegans may also be used in scientific approaches. Non-limiting examples of agronomically important animals are sheep, cattle and pigs, while, for example, cats and dogs may be considered as economically important animals. Preferably, the subject/patient is a mammal; more preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orang-utan, a gibbon, a sheep, cattle, or a pig); most preferably, the subject/patient is a human.

Non-Medical Use of the Compounds of the Present Invention

The present invention also relates to compositions comprising any one of the compounds of the present invention for uses other than in medicine. Such non-therapeutic use may, for example, be as a cosmetic, sun protectant, food, drink, flavoring, animal feed or dietary supplement, but is not limited thereto.

Such compositions according to the present invention may be in any form, and are preferably in the form of a food, drink, animal feed, cosmetic, sun-protectant, flavoring, or dietary supplement.

In the non-medical applications, the compounds according to the present invention may be in the form of cosmetically or nutritionally acceptable salts which are as defined for the pharmaceutically acceptable salts, solvates or prodrugs.

The compounds of the present invention are particularly suitable for promoting hair growth and as agents for anti-aging, anti-wrinkle, anti-pollution and as anti-oxidants. Anti-pollution agents can, e.g., be suitably used for preventing damage caused by UV-radiation and environmental pollutants such as particles present in exhaust gases.

Furthermore, the compounds of the present invention promote collagen synthesis and/or fibronectin synthesis which supports a firm skin, reduces wrinkles and diminishes skin aging. In addition, the compounds of the present invention promote wound healing.

The compounds and compositions described herein are therefore preferably used in order to promote hair growth and wound healing. In particular, the non-therapeutic use of the compounds and/or compositions described herein as a cosmetic, sun protectant, food, drink, flavouring, animal feed or dietary supplement preferably promotes hair growth and wound healing.

Preparation of the Compounds of the Present Invention

Compounds of the present invention may be prepared by a method comprising the steps of incubating/contacting a flavonoid as defined herein with a glycosyl transferase and obtaining the compound of the present invention. Thus, in order to prepare the compounds of the present invention, it is preferred to use a glycosyl transferase for efficient production. In principle, any glycosyl transferase may be used. However, it is preferred that a glycosyl transferase belonging to family GT1 is used. In this regard, the glycosyl transferases GTC, GTD and GTF belong to the glycosyltransferase family GT1 (EC 2.4.1.x) (Coutinho (2003) JMB 328(2):307-317). This family comprises enzymes that mediate sugar transfer to small lipophilic acceptors. Family GT1 members uniquely possess a GT-B fold. They catalyze an inverting reaction mechanism concerning the glycosidic linkage in the sugar donor and the formed one in the acceptor conjugate, creating natural β-D- or α-L-glycosides.

Within the GT-B fold the enzymes form two major domains, one N-terminal and a C-terminal, with a linker region in between. Generally, the N-terminus constitutes the AA-residues responsible for acceptor binding and the residues determining donor binding are mainly located in the C-terminus. In family GT1 the C-terminus contains a highly conserved motif possessing the AA residues that take part in nucleoside-diphosphate (NDP)-sugar binding. This motif was also termed the plant secondary product glycosyltransferase (PSPG) box (Hughes (1994) Mit DNA 5(1):41-49.

Flavonoid GTs belong to family GT1. Due to the natural biosynthesis of flavonoids in plants most of the enzymes are also known from plants. However, several enzymes from the other eukaryotic kingdoms fungi and animals and also from the domain of bacteria are described. In eucarya, sugar donors of GT1 enzymes are generally uridinyl-diphosphate (UDP)-activated. Of these so called UGTs or UDPGTs, most enzymes transfer glucose residues from UDP-glucose to the flavonoid acceptors. Other biological relevant sugars from UDP-galactose, -rhamnose, -xylose, -arabinose, and -glucuronic acid are less often transferred.

Also several bacterial GT1s were discovered that are able to glycosylate also flavonoid acceptors. These enzymes all belong to the GT1 subfamily of antibiotic macrolide GTs (MGT). In bacteria GT1 enzymes use UDP-glucose or -galactose but also deoxythymidinyl-diphosphate (dTDP)-activated sugars as donor substrates. However, all the bacterial flavonoid active GT1 enzymes have UDP-glucose as the native donor. There is only one known exception with the metagenome derived enzyme GtfC that was the first bacterial GT1 reported to transfer rhamnose to flavonoids (Rabausch (2013) Appl Environ Microbiol 79(15):4551-4563). However, until the present disclosure and as shown in the appended Examples, it was established that this activity is limited to C3-OH or the C7-OH groups of flavonoids. Transfer to the C3′-OH and the C4′-OH of the flavonoid C-ring was already less commonly observed. Other positions are rarely glycosylated, if at all. Specifically, there are only few examples concerning the glycosylation of the C5-OH group, which is based on the fact that this group is sterically protected. Therefore, the only examples relate to anthcyanidins (Janvary (2009) J Agric Food Chem 57(9):3512-3518; Lorenc-Kukala (2005) J Agric Food Chem 53(2):272-281; Tohge (2005) The Plant J 42(2):218-235). This class of flavonoids lacks the C4 keto group which facilitates nucleophilic attack. The C5-OH group of (iso)flavones and (iso)flavanones is protected through hydrogen bridges with the neighbored carbonyl group at C4. This was thought to even hinder chemical glycosylation approaches at C5 of these classes.

Today, there are only three GT1 enzymes characterized that create 5-O-β-D-glucosides of flavones. One is UGT71G1 from Medicago truncatula which was proven to be not regio-selective and showed a slight side activity in glucosylation of C5-OH on quercetin (He (2006) JBC 281(45):34441-7. An exceptional UGT was identified in the silkworm Bombyx mori capable of specifically forming quercetin-5-O-β-D-glucoside (Daimon (2010) PNAS 107(25):11471-11476; Xu (2013) Mol Biol Rep 40(5):3631-3639). Finally, a mutated variant of MGT from Streptomyces lividans presented low activity at C5-OH of 5-hydroxyflavone after single AA exchange (Xie (2013) Biochemistry (Mosc) 78(5):536-541). However, the wild type MGT did not possess this ability nor did other MGTs.

Flavanol-5-O-α-D-glucosides were synthesized through transglucosylation activity of hydrolases, i.e. α-amylases (EC 3.2.1.x) (Noguchi (2008) J Agric Food Chem 56(24):12016-12024; Shimoda (2010) Nutrients 2(2):171-180). However, the flavonols also lack the C4=O-group and the enzymes create a “non-natural” α-D-glucosidic linkage.

It is noteworthy that all so far known 5-O-GTs mediated only glucosylation. The prior art is entirely silent with regard to rhamnosylation of flavonoids, much less using the method as disclosed herein above and as shown in the appended Examples.

Flavonoids are secondary metabolites, predominantly of higher plants. Thus, flavonoids are commonly extracted from plant matrices. Used methods for the extraction are the conventional liquid-liquid or solid-liquid extractions with organic solvents, e.g. hexane, acetone, ethyl acetate or methanol. More advanced processes employ pressurized liquid extraction, subcritical and supercritical extractions, and microwave- and ultrasound-assisted extractions Gil-Chivez et al. 2013, Compr. Rev. Food Sci Food Safety, 12:5-23, doi: 10.1111/1541-4337.12005). Other technologies to synthesize flavonoids are biotechnological approaches with metabolically engineered microorganisms as yeasts or bacteria (Trantas et al. 2015, Front Plant Sci 6:7, doi: 10.3389/fpls.2015.00007). Product yields of biotechnological processes generally still not reach industrial profitability. Chemical synthesis also is a valuable technology (Selepe et al. 2013, Molecules 18_4739-4765, doi:10.3390/molecules 18044739). At least some chemical processes for specific classes of flavonoids are described, e.g., for anthcyanins (WO 2006/134352 A1).

It is to be understood that the present invention specifically relates to each and every combination of features and examples described herein, including any combination of general and/or preferred features/examples. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLES

The compounds described in this section are defined by their chemical formulae and their corresponding chemical names. In case of conflict between any chemical formula and the corresponding chemical name indicated herein, the present invention relates to both the compound defined by the chemical formula and the compound defined by the chemical name.

Part A: Preparation of 5-O-rhamnosylated Flavonoids

### Example A1—Preparation of Media and Buffers

The methods of the present invention can be used to produce rhamnosylated flavonoids, as will be shown in the appended Examples.

Several growth and biotransformation media were used for the rhmanoslyation of flavonoids. Suitable media thus include: Rich Medium (RM) (Bacto peptone (Difco) 10 g, Yeast extract 5 g, Casamino acids (Difco) 5 g, Meat extract (Difco) 2 g, Malt extract (Difco) 5 g, Glycerol 2 g, MgSO₄×7 H₂O 1 g, Tween 80 0.05 g and H₂O ad 1000 mL at a final pH of about 7.2); Mineral Salt Medium (MSM) (Buffer and mineral salt stock solution were autoclaved. After the solutions had cooled down, 100 mL of each stock solution were joined and 1 mL vitamin and 1 mL trace element stock solution were added. Then sterile water was added to a final volume of 1 L. The stock solutions were: Buffer stock solution (10×) of Na₂HPO₄ 70 g, KH₂PO₄ 20 g and H₂O ad 1000 mL; Mineral salt stock solution (10×) of (NH₄)₂SO₄ 10 g, MgCl₂×6 H₂O 2 g, Ca(NO₃)₂×4 H₂O 1 g and H₂O ad 1000 mL; Trace element stock solution (1000×) of EDTA 500 mg, FeSO₄×7 H₂O 300 mg, CoCl₂×6 H₂O 5 mg, ZnSO₄×7 H₂O 5 mg, MnCl₂×4 H₂O 3 mg, NaMoO₄×2 H₂O 3 mg, NiCl₂×6 H₂O 2 mg, H₃BO₃ 2 mg, CuCl₂×2 H₂O 1 mg and H₂O ad 200 mL. The solution was sterile filtered. Vitamin stock solution (1000×) of Ca-Pantothenate 10 mg, Cyanocobalamine 10 mg, Nicotinic acid 10 mg, Pyridoxal-HCl 10 mg, Riboflavin 10 mg, Thiamin-HCl 10 mg, Biotin 1 mg, Folic acid 1 mg, p-Amino benzoic acid 1 mg and H₂O ad 100 mL. The solution was sterile filtered.); Lysogeny Broth (LB) (Yeast extract 5 g, Peptone 10 g, NaCl 5 g and H₂O ad 1000 mL); Terrific Broth (TB) (casein 12 g, yeast extract 24 g, K₂HPO₄ 12.5 g, KH₂PO₄ 2.3 g and H₂O ad 1000 mL at pH 7.2). In some experiments, in particular when the concentration of dissolved oxygen (DO) was above about 50%, nutrients were added to the solution. This was done using a feed solution of Glucose 500 g, MgSO₄ 10 g, thiamine 1 mg and H₂O ad 1000 mL. In some experiments, in particular when cells expressing glycosyl transferase were harvested prior to starting the production of rhamnosylated flavonoids, cells were resuspended in a buffer solution, in particular phosphate buffer saline (PBS). The solution was prepared using NaCl 150 mM, K₂HPO₄/KH₂PO₄ 100 mM at a pH of 6.4 to 7.4.

### Example A2—Glycosyl Transferases Used for the Production of Rhamnosylated Flavonoids

Several different glycosyl transferases were used in the methods of the present invention to produce rhamnosylated flavonoids. In particular, the glycosyltransferases (GTs) used for flavonoid rhamnoside production were
1. GTC, a GT derived metagenomically (AGH18139), preferably having an amino acid sequence as shown in SEQ ID NO:3, encoded by a polynucleotide as shown in SEQ ID NO:4. A codon-optimized sequence for expression in E. coli is shown in SEQ ID NO:27.
2. GTD, a GT from Dyadobacter fermentans (WP_015811417), preferably having an amino acid sequence as shown in SEQ ID NO:5, encoded by a polynucleotide as shown in SEQ ID NO:6. A codon-optimized sequence for expression in E. coli is shown in SEQ ID NO:28.
3. GTF, a GT from Fibrisoma limi (WP_009280674), preferably having an amino acid sequence as shown in SEQ ID NO:7, encoded by a polynucleotide as shown in SEQ ID NO:8. A codon-optimized sequence for expression in E. coli is shown in SEQ ID NO:29.
4. GTS from Segetibacter koreensis (WP_018611930) preferably having an amino acid sequence as shown in SEQ ID NO:9, encoded by a polynucleotide as shown in SEQ ID NO: 10. A codon-optimized sequence for expression in E. coli is shown in SEQ ID NO:30.
5. Chimera 3 with AAs 1 to 316 of GTD and AAs 324 to 459 of GTC preferably having an amino acid sequence as shown in SEQ ID NO: 58, encoded by a polynucleotide as shown in SEQ ID NO: 59. A codon-optimized sequence for expression in E. coli is shown in SEQ ID NO: 60.
6. Chimera 4 with AAs 1 to 268 of GTD and AAs 276 to 459 of GTC preferably having an amino acid sequence as shown in SEQ ID NO: 61, encoded by a polynucleotide as shown in SEQ ID NO: 62. A codon-optimized sequence for expression in E. coli is shown in SEQ ID NO: 63.
7. Chimera 1 frameshift with AAs 1 to 234 of GTD and AAs 242 to 443 of GTC preferably having an amino acid sequence as shown in SEQ ID NO: 23, encoded by a polynucleotide as shown in SEQ ID NO: 24.

The GT genes were amplified by PCR using respective primers given in Table A1. Purified PCR products were ligated into TA-cloning vector pDrive (Qiagen, Germany). Chemically competent E. coli DH5a were transformed with ligation reactions by heat shock and positive clones verified by blue/white screening after incubation. GT from Segetibacter koreensis was directly used as codon-optimized nucleotide sequence.

Chimera 3 and chimera 4 were created from the codon-optimized nucleotide sequences from GTD and GTC, while chimera 1 was constructed from the SEQ ID NO:4 and SEQ ID NO:6. Chimera 1 was created according to the ligase cycling reaction method described by Kok (2014) ACS Synth Biol 3(2):97-106. Thus, the two nucleotide sequences of each chimeric fragment were amplified via PCR and were assembled using a single-stranded bridging oligo which is complementary to the ends of neighboring nucleotide parts of both fragments. A thermostable ligase was used to join the nucleotides to generate the full-length sequence of the chimeric enzyme.

Chimera 3 and chimera 4 were constructed according to the AQUA cloning method described by Beyer (2015) PLoS ONE 10(9):e0137652. Therefore, the nucleotide fragments were amplified with complementary regions of 20 to 25 nucleotides, agarose-gel purified, mixed in water, incubated for 1 hour at room temperature and transformed into chemically competent E. coli DH5α. The primers used for the chimera construction are listed in Table A2.

TABLE A1
Primers used for the amplification of the GT genes
by PCR

| Enzyme | Primer name | Sequence (5′ → 3′) |
|---|---|---|
| GTC | GTC-NdeI-for | CATATGAGTAATTTATTTTCTTCACAAAC |
| | GTC-BamHI-rev | GGATCCTTAGTATATCTTTTCTTCTTC |
| GTD | GTF-XhoI_for | CTCGAGATGACGAAATACAAAAATGAAT |
| | GTF_BamHI_rev | GGATCCTTAACCGCAAACAACCCGC |
| GTF | GTL_XhoI_for | CTCGAGATGACAACTAAAAAAATCCTGTT |
| | GTL_BamHI_rev | GGATCCTTAGATTGCTTCTACGGCTT |
| GTS | GTSopt_pET_fw | GGGAATTCCATATGATGAAATATATCAGC |
| | | TCCATTCAG |
| | GTSopt_pET_rv | CGGGATCCTTAAACCAGAACTTCGGCCTG |
| | | ATAG |

TABLE A2
Primers used for the construction of chimeric enzymes

| Enzyme | Primer name | Sequence (5′ → 3′) |
|---|---|---|
| Chimera 1 | Bridge_P1_pETGTD | GCGGCCATATCGACGACGACGACAAGCATATGACGA |
| | | AATACAAAAATGAATTAACAGGT |
| | Bridge_P1_GTCpET | GGAAGAAGAAAAGATATACTAAGGATCCGGCTGCTAA |
| | | CAAAGCCCGAAAGG |
| | Chim_P1_D_Nde_for | CATATGACGAAATACAAAAATGAATT |
| | Chim_P1_D_rev | GCGGTCATACTCAAATGATT |
| | Chim_P1_C_for | AGTGATCTGGGAAAAAATATC |
| | Chim_P1_C_Bam_rev | GGATCCTTAGTATATCTTTTCTTCTTCCT |
| Chimera 3 | GTDopt_pEt_fw | GGGAATTCCATATGATGACCAAATACAAAAATG |
| | Chim3_pET_rv | CGGGATCCTTAGTAAATCTTTTCTTCTTCCTTC |
| | 1-Chim3-opt-o(Chim3- | TGCCCTGAGGAAAGCGCGCACGTAATTC |
| | opt) | |
| | 2f-Chim3-opt-o(Chim3- | TGCGCGCTTTCCTCAGGGCAACTTAATC |
| | opt) | |
| | 1f-Assembly-o(Vec) | TGACGATAAGGATCGATGGGGATCCATGACCAAATAC |
| | | AAA |
| | 1r-Assembly-o(Vec) | TATGGTACCAGCTGCAGATCTCGAGTTAGTAAATCTTT |
| | | TCTTC |
| Chimera 4 | GTDopt_pEt_fw | GGGAATTCCATATGATGACCAAATACAAAAATG |
| | Chim3_pET_rv | CGGGATCCTTAGTAAATCTTTTCTTCTTCCTTC |
| | 1r-Chim4_GTD- | CGATTTTGCGCCCATATTGTAACAACTTTTGA |
| | o(Chim4_GTC) | |
| | 2f-Chim4_GTC- | ACAATATGGGCGCAAAATCGTCGTAGTC |
| | o(Chim4_GTD) | |
| | 1f-Assembly-o(Vec) | TGACGATAAGGATCGATGGGGATCCATGACCAAATAC |
| | | AAA |
| | 1r-Assembly-o(Vec) | TATGGTACCAGCTGCAGATCTCGAGTTAGTAAATCTTT |
| | | TCTTC |

To establish expression hosts purified pDrive::GT vectors were incubated with respective endonucleases (Table A1) and the fragments of interest were purified from Agarose after gel electrophoresis. Alternatively, the amplified and purified PCR product was directly incubated with respective endonucleases and purified from agarose gel after electrophoresis. The fragments were ligated into prepared pET19b or pTrcHisA plasmids and competent E. coli Rosetta gami 2 (DE3) were transformed by heat shock. Positive clones were verified after overnight growth by direct colony PCR using T7 promotor primers and the GT gene reverse primers, respectively.

Altogether, seven production strains were established:

| | |
|---|---|
| 1. PetC | E. coli Rosetta gami 2 (DE3) pET19b::GTC |
| 2. PetD | E. coli Rosetta gami 2 (DE3) pET19b::GTD |
| 3. PetF | E. coli Rosetta gami 2 (DE3) pET19b::GTF |
| 4. PetS | E. coli Rosetta gami 2 (DE3) pET19b::GTS |
| 5. PetChim1fs | E. coli Rosetta gami 2 (DE3) pET19b::Chimera 1 |
| | frameshift |
| 6. PetChim3 | E. coli Rosetta gami 2 (DE3) pET19b::Chimera 3 |
| 7. PetChim4 | E. coli Rosetta gami 2 (DE3) pET19b::Chimera 4 |

### Example A3—Production of Rhamnosylated Flavonoids in Biotransformations

Three kinds of whole cell bioconversion (biotransformation) were performed. All cultures were inoculated 1/100 with overnight pre-cultures of the respective strain. Pre-cultures were grown at 37° C. in adequate media and volumes from 5 to 100 mL supplemented with appropriate antibiotics.

1. Analytical Small Scale and Quantitative Shake Flask Cultures

For analytical activity evaluations, 20 mL biotransformations were performed in 100 mL Erlenmeyer flasks while quantitative biotransformations were performed in 500 mL cultures in 3 L Erlenmeyer flasks. Bacterial growth was accomplished in complex media, e.g. LB, TB, and RM, or in M9 supplemented with appropriate antibiotics at 28° C. until an OD₆₀₀ of 0.8. Supplementation of 50 or 100 μM Isopropyl-β-D-thiogalactopyranoside (IPTG) induced gene expression overnight (16 h) at 17° C. and 175 rpm shaking. Subsequently, a polyphenolic substrate, e.g. Naringenin, Hesperetin or else, in concentrations of 200-800 μM was added to the culture. Alternatively, the polyphenolic substrate was supplemented directly with the IPTG. A third alternative was to harvest the expression cultures by mild centrifugation (5.000 g, 18° C., 10 min) and suspend in the same volume of PBS, supplied with 1% (w/v) glucose, optionally biotin and/or thiamin, each at 1 mg/L, the appropriate antibiotic and the substrate in above mentioned concentrations. All biotransformation reactions in 3 L shake flasks were incubated at 28° C. up to 48 h at 175 rpm.

2. Quantitative Bioreactor (Fermenter) Cultures

In order of a monitorable process bioconversions were performed in volumes of 0.5 L in a Dasgip fermenter system (Eppendorf, Germany). The whole process was run at 26 to 28° C. and kept at pH 7.0. The dissolved oxygen (DO) was kept at 30% minimum. During growth the DO rises due to carbohydrate consumption. At DO of 50% an additional feed with glucose was started with 1 mL/h following the equation

y=e^{0.1x}

whereby y represents the added volume (mL) and x the time (h).

For cell growth the bacterial strains were grown in LB, TB, RM or M9 overnight. At OD₆₀₀ of 10 to 50 50 μM of IPTG and the polyphenolic substrate (400-1500 μM) were added to the culture. The reaction was run for 24 to 48 h.

All bioconversion reactions were either stopped by cell harvest through centrifugation (13,000 g, 4° C., 20 min) followed by sterile filtration with a 0.22 μM PES membrane (Steritop™, Carl Roth, Germany). Alternatively, cultures were harvested by hollow fibre membrane filtration techniques, e.g. TFF Centramed system (Pall, USA). Supernatants were purified directly or stored short-term at 4° C. (without light).

Qualitative Analyses of Biotransformation Reactions and Products

Biotransformation products were determined by thin layer chromatography (TLC) or by HPLC.

For qualitative TLC analysis, 1 mL culture supernatant was extracted with an equal amount ethyl acetate (EtOAc). After centrifugation (5 min, 3,000 g) the organic phase was transferred into HPLC flat bottom vials and was used for TLC analysis. Samples of 20 μL were applied on 20×10 cm² (HP)TLC silica 60 F₂₅₄ plates (Merck KGaA, Darmstadt, Germany) versus 200 pmol of reference flavonoids by the ATS 4 (CAMAG, Switzerland). To avoid carryover of substances, i.e. prevent false positives, samples were spotted with double syringe rinsing in between. The sampled TLC plates were developed in EtOAc/acetic acid/formic acid/water (EtOAc/HAc/HFo/H₂O) 100:11:11:27. After separation the TLC plates were dried in hot air for 1 minute. The chromatograms were read and absorbances of the separated bands were determined densitometrically depending on the absorbance maximum of the educts at 285 to 370 nm (D2) by a TLC Scanner 3 (CAMAG, Switzerland).

Analytical HPLC Conditions

HPLC analytics were performed on a VWR Hitachi LaChrom Elite device equipped with diode array detection.
Column: Agilent Zorbax SB-C18 250×4.6 mm, 5 μM
Flowrate: 1 mL/min
Mobile phases: A: H₂O+0.1% Trifluoro acetic acid (TFA), B: ACN+0.1% TFA
Gradient: 0-5′:5% B, 5-15′: 15% B, 15-25′: 25% B, 25-25′: 35% B, 35-45′: 40%, 45-55′ 100% B, 55-63′: 5% B
Sample injection volume 100-500 μL

MS and MS/MS analyses were obtained on a microOTOF-Q with electrospray ionization (ESI) from Bruker (Bremen, Germany). The ESI source was operated at 4000 V in negative ion mode. Samples were injected by a syringe pump and a flow rate of 200 μL/min.

In order to purify the polyphenolic glycosides two different purification procedures were applied successfully.
1. Extraction and subsequent preparative HPLC
   1.1 In liquid-liquid extractions bioconversion culture supernatants were extracted twice with half a volume of iso-butanol or EtOAc.
   1.2 In solid phase extractions (SPE) supernatants were first bound on suitable polymeric matrices, e.g. Amberlite XAD resins or silica based functionalized phases, e.g. C-18, and subsequently eluted with organic solvents, e.g. ACN, methanol (MeOH), EtOAc, dimethyl sulfoxide (DMSO) et al. or with suitable aqueous solutions thereof, respectively.
   Organic solvents were evaporated and the residuum completely dissolved in water-acetonitrile (H₂O-ACN) 80:20. This concentrate was further processed by HPLC as described below.
2. Direct fractionation by preparative HPLC
   Sterile filtered (0.2 μm) biotransformation culture supernatants or pre-concentrated extracts were loaded on adequate RP18 columns (5 μm, 250 mm) and fractionated in a H₂O-ACN gradient under following general conditions:

| System: | Agilent 1260 Infinity HPLC system. | |
|---|---|---|
| Column: | ZORBAX SB-C18 prepHT 250 × 21.2 mm, 7 μm. | |
| Flowrate: | 20 mL/min | |
| Mobile Phase: | A: Water + 0.1 formic acid | |
| | B: ACN + 0.1 formic acid | |
| Gradient: | 0-5 min | 5-30% B |
| | 5-10 min | 30% B |
| | 10-15 min | 35% B |
| | 15-20 min | 40% B |
| | 20-25 min | 100% B |

Fractions containing the polyphenolic glycosides were evaporated and/or freeze dried. Second polishing steps were performed with a pentafluor-phenyl (PFP) phase by HPLC to separate double peaks or impurities.

The rhamnose transferring activity was shown with enzymes GTC, GTD, GTF and GTS and the three chimeric enzymes chimera 1 frameshift, chimera 3 and chimera 4 in preparative and analytical biotransformation reactions. The enzymes were functional when expressed in different vector systems. GT-activity could be already determined in cloning systems, e.g. E. coli DH5a transformed with pDrive vector (Qiagen, Germany) carrying GT-genes. E. coli carrying pBluescript II SK+ with inserted GT-genes also was actively glycosylating flavonoids. For preparative scales the production strains PetC, PetD, PetF, PetS, PetChim1fs, PetChim3 and PetChim4 were successfully employed. Products were determined by HPLC, TLC, LC-MS and NMR analyses.

Biotransformation of the Flavanone Hesperetin Using E. coli Rosetta Gami 2 (DE3) pET19b::GTC (PetC)

In a preparative scale reaction hesperetin (3′,5,7-Trihydroxy-4′-methoxyflavanone, 2,3-dihydro-5,7-dihydroxy-2-(3-hydroxy-4-methoxyphenyl)-4H-1-benzopyran-4-one, CAS No. 520-33-2) was converted. The biotransformation was performed following general preparative shake flask growth and bioconversion conditions.

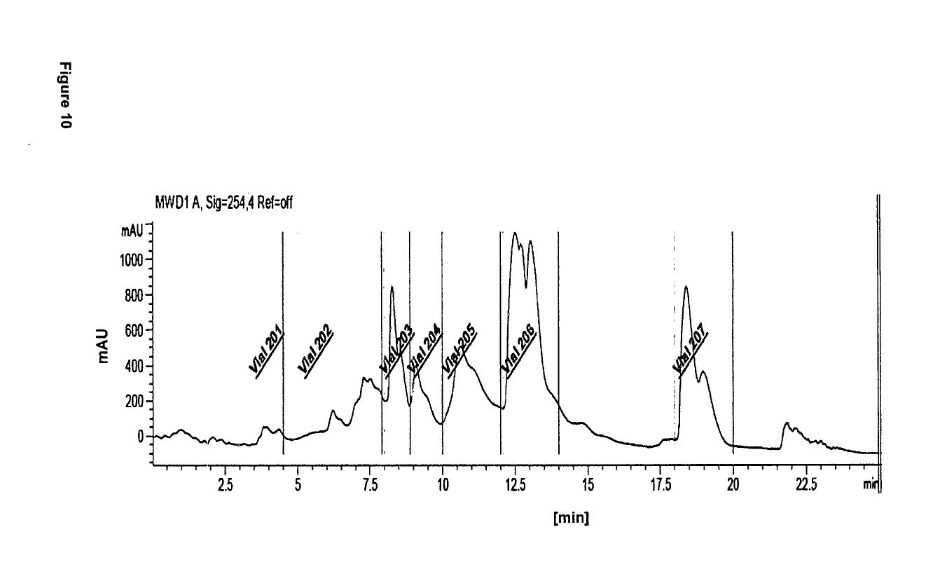

The bioconversion of hesperetin (>98%, Cayman, USA) was monitored by HPLC analyses of 500 μL samples taken at start (T=0), 3 h and 24 h reaction at 28° C. The culture supernatant was loaded directly via pump flow to a preparative RP18 column (Agilent, USA). Stepwise elution was performed and seven fractions were collected according to FIG. 10 and table A2.

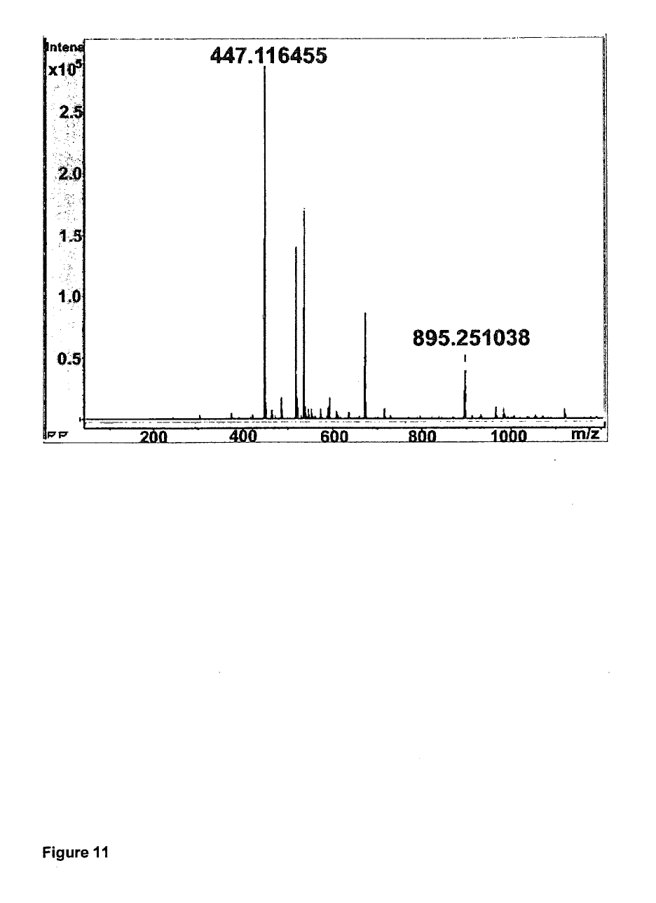

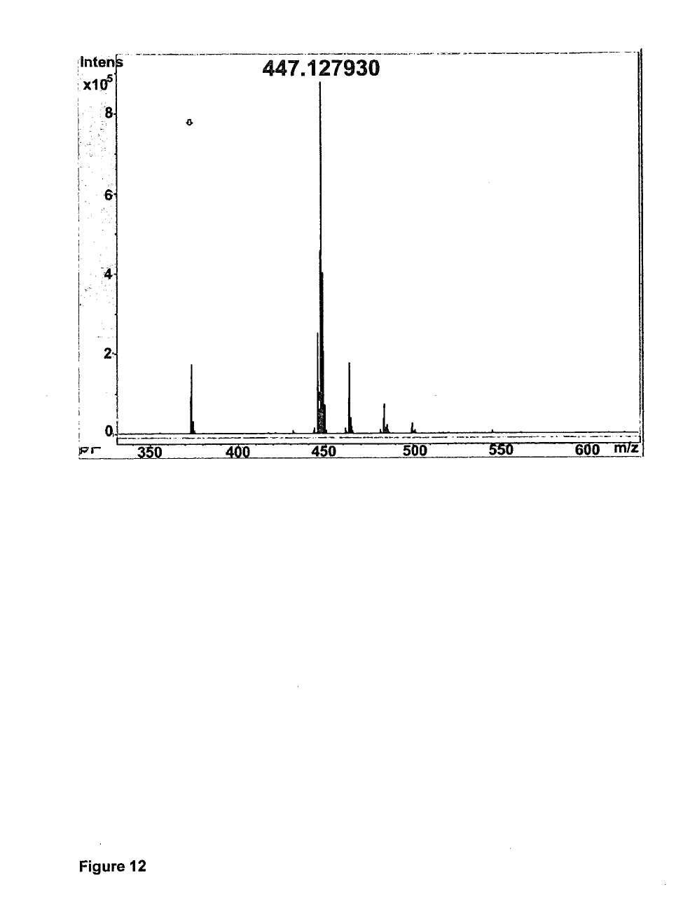

All seven fractions subsequently were analyzed by HPLC and ESI-Q-TOF MS analyses. MS analyses in negative ion mode revealed fraction 3 and fraction 6 to contain a compound each with the molecular weight of 448 Da corresponding to hesperetin-O-rhamnoside (C₂₂H₂₄O₁₀) (FIGS. 11 and 12 table A2). To further purify the two compounds fractions 3 and 6 were lyophilized and dissolved in 30% ACN.

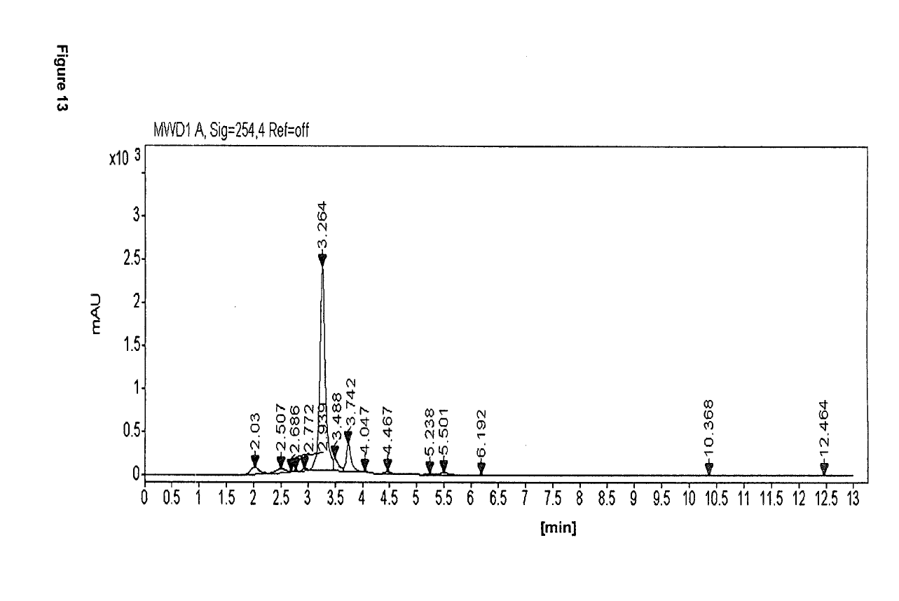

Final purification was performed by HPLC using a PFP column The second purification occurred on a Hypersil Gold PFP, 250×10 mm, 5 μm purchased from Thermo Fischer Scientific (Langerwehe, Germany) and operated at a flow rate of 6 mL/min (Mobile Phase: A: Water, B: ACN, linear gradient elution (0′-8′:95%-40% A, 8′-13′:100% B) (FIG. 13). Subsequently, ESI-TOF MS analyses of the PFP fractions identified the target compounds designated HESR1 and HESR2 in respective fractions (table A3).

After lyophilization NMR analyses elucidated the molecular structure of HESR1 and HESR2, respectively (Example B-2). HESR1 turned out to be the hesperetin-5-O-α-L-rhamnoside and had a RT of 28.91 min in analytical HPLC conditions. To this point, this compound has ever been isolated nor synthesized before.

TABLE A2
Fractionation of hesperetin bioconversion by prepLC separation

| Frac | Well | | Volume | BeginTime | EndTime | | |
|---|---|---|---|---|---|---|---|
| # | # | Location | [μl] | [min] | [min] | Description | ESI-MS |
| 1 1 | Vial | 201 | 20004.17 | 3.4999 | 4.5001 Time | | |
| 2 1 | Vial | 202 | 58004.17 | 4.9999 | 7.9001 Time | | |
| 3 1 | Vial | 203 | 17804.17 | 7.9999 | 8.8901 Time | HESR1 | 448 |
| 4 1 | Vial | 204 | 20791.67 | 8.9505 | 9.9901 Time | | |
| 5 1 | Vial | 205 | 39012.50 | 10.0495 | 12.0001 Time | | |
| 6 1 | Vial | 206 | 38004.17 | 12.0999 | 14.0001 Time | HESR2 | 448 |
| 7 1 | Vial | 207 | 40004.17 | 17.9999 | 20.0001 Time | | |

Biotransformation of the Flavanone Naringenin Using PetC in a Preparative Shake Flask Culture

Naringenin (4′,5,7-Trihydroxyflavanone, 2,3-dihydro-5,7-dihydroxy-2-(4-hydroxyphenyl)-4H-1-benzopyran-4-one, CAS No. 67604-48-2) was converted in a preparative scale reaction. The biotransformation was performed following general preparative shake flask growth and bioconversion conditions.

The bioconversion of naringenin (98%, Sigma-Aldrich, Switzerland) was controlled by HPLC analyses of a 500 μL sample after 24 h reaction. The culture supernatant was directly loaded via pump flow to a preparative RP18 column. Stepwise elution was performed and seven fractions were collected according to table A4.

All seven fractions subsequently were analyzed by HPLC and ESI-TOF MS analyses. MS analyses in negative ion mode revealed fraction 3 and fraction 5 to contain a compound each with the molecular weight of 418 Da which is the molecular weight of naringenin-O-rhamnoside (C₂₁H₂₂O₉)(table A4). The two compounds designated NR1 and NR2 were lyophilized. HPLC analysis in analytical conditions revealed RTs of approx. 27.2 min for NR1 and 35.7 min for NR2, respectively. NMR analyses elucidated the molecular structure of NR1 (Example B-3). NR1 was identified to be an enantiomeric 1:1 mixture of S- and R-naringenin-5-O-α-L-rhamnoside (N5R). Since the used precursor also was composed of both enantiomers the structure analysis proved that both isomers were converted by GTC. To our knowledge this is the first report that naringenin-5-O-α-L-rhamnoside has ever been biosynthesized. The compound was isolated from plant material (Shrivastava (1982) Ind J Chem Sect B 21(6):406-407). However, the rare natural occurrence of this scarce flavonoid glycoside has impeded any attempt of an industrial application.

In contrast, the first time bioconversion of naringenin-5-O-α-L-rhamnoside opens the way of a biotechnological production process for this compound. Until now the biotechnological production was only shown for e.g. naringenin-7-O-α-L-xyloside and naringenin-4′-O-β-D-glucoside (Simkhada (2009) Mol. Cells 28:397-401, Werner (2010) Bioprocess Biosyst Eng 33:863-871).

TABLE A4
Fractionation of naringenin bioconversion by prepLC separation

| Frac | Well | | Volume | BeginTime | EndTime | | |
|---|---|---|---|---|---|---|---|
| # | # | Location | [μl] | [min] | [min] | Description | ESI-MS |
| 1 1 | Vial | 201 | 31518.75 | 4.6963 | 6.4407 Time | | |
| 2 1 | Vial | 202 | 17328.75 | 6.5074 | 7.4634 Time | | |
| 3 1 | Vial | 203 | 34638.75 | 7.5301 | 9.4478 Time | NR1 | 418 |
| 4 1 | Vial | 204 | 43905.00 | 9.5130 | 11.9455 Time | | |
| 5 1 | Vial | 205 | 115995.00 | 12.0109 | 18.4484 Time | NR2 | 418 |
| 6 1 | Vial | 206 | 71111.25 | 18.5151 | 22.4590 Time | | |
| 7 1 | Vial | 207 | 80047.50 | 22.5242 | 26.9647 Time | | |

Biotransformation of Naringenin Using E. coli Rosetta Gami 2 (DE3) pET19b::GTC (PetC) in a Monitored Bioreactor System

Next to production of naringenin rhamnosides in shake flask cultures a bioreactor process was successfully established to demonstrate applicability of scale-up under monitored culture parameters.

In a Dasgip fermenter system (Eppendorf, Germany) naringenin was converted in four fermenter units in parallel under conditions stated above.

At an OD₆₀₀ of 50 expression in PetC was induced by IPTG while simultaneously supplementation of 0.4 g of naringenin (98% CAS No. 67604-48-2, Sigma-Aldrich, Switzerland) per unit was performed. Thus, the final concentration was 2.94 mM of substrate.

After bioconversion for 24 h the biotransformation was finished and centrifuged. Subsequently, the cell free supernatant was extracted once with an equal volume of iso-butanol by shaking intensively for one minute. Preliminary extraction experiments with defined concentrations of naringenin rhamnosides revealed an average efficiency of 78.67% (table A5).

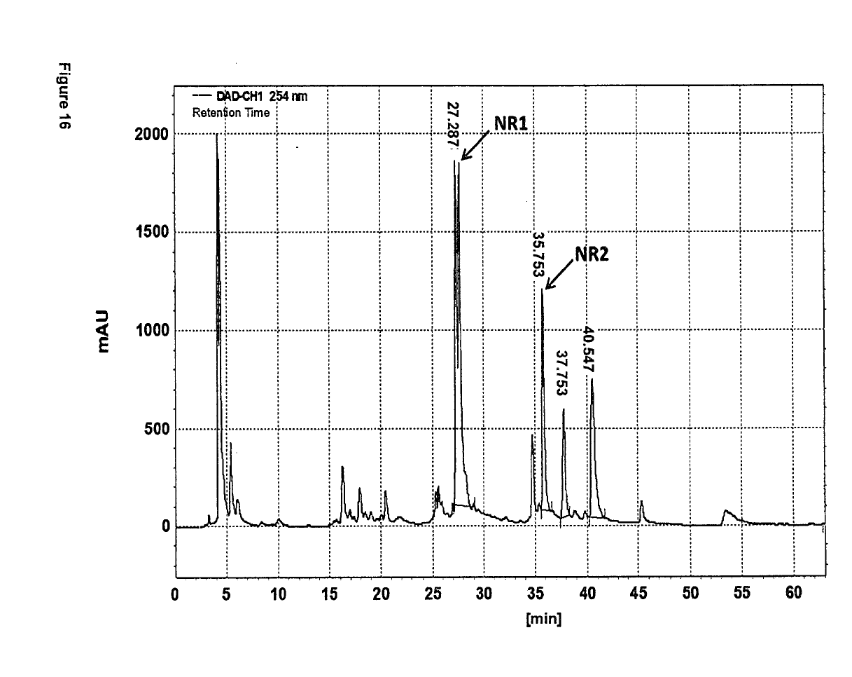

HPLC analyses of the bioreactor reactions indicated that both products, NR1 (RT 27,28′) and NR2 (RT 35.7′), were built successfully (FIG. 16). ESI-MS analyses verified the molecular mass of 418 Da for both products. Quantitative analysis of the bioconversion products elucidated the reaction yields. Concentration calculations were done from peak areas after determination regression curves of NR1 and NR2 (table A6). NR1 yielded an average product concentration of 393 mg/L, NR2 as the byproduct yielded an average 105 mg/L.

TABLE A5
Extraction of naringenin biotransformation products
from supernatant with iso-butanol

| | |
|---|---|
| Extraction mit iso-butanol | 1 ml/1 mL 1′ shaking |

| % | Mean | Loss % | Std Dev. |
|---|---|---|---|
| 75,75160033 | | | |
| 82,49563254 | 78,6707143 | 21,32928571 | 2,73747541 |
| 76,42705533 | | | |
| 80,00856895 | | | |

Biotransformation of narengenin using E. coli Rosetta gami 2 (DE3) pET19b::GTC (PetC), E. coli Rosetta gami 2 (DE3) pET19b::GTD (PetD), E. coli Rosetta gami 2 (DE3) pET19b::GTF (PetF), E. coli Rosetta gami 2 (DE3) pET19b::GTS (PetS), E. coli Rosetta gami 2 (DE3) pET19b::Chimera 1 frameshift (PetChim1fs), E. coli Rosetta gami 2 (DE3) pET19b::Chimera 3 (PetChim3) and E. coli Rosetta gami 2 (DE3) pET19b::Chimera 4 (PetChim4), respectively

To determine the regio specificities of GTC, GTD, GTF and GTS as well as the three chimeric enzymes chimera 1 frameshift, chimera 3 and chimera 4 biotransformations were performed in 20 mL cultures analogously to preparative flask culture bioconversions using naringenin as a substrate among others. To purify the formed flavonoid rhamnosides, the supernatant of the biotransformation was loaded on a C₆H₅ solid phase extraction (SPE) column. The matrix was washed once with 20% acetonitrile. The flavonoid rhamnosides were eluted with 100% aceteonitrile. Analyses of the biotransformations were performed using analytical HPLC and LC-MS. For naringenin biotransformations analyses results of the formed products NR1 and NR2 of each production strain are listed in Table A7 and A8, respectively.

TABLE A7
Formed NR1 products in bioconversions of
naringenin with different production strains

| strain | NR1 retention time [min] HPLC | ESI-MS | ESI-MSMS |
|---|---|---|---|
| PetC | 27.32 | 418 | 272 |
| PetD | 27.027 | 418 | 272 |
| PetF | 26.627 | 418 | 272 |
| PetS | 26.833 | 418 | 272 |
| PetChim1fs | 26.673 | 418 | 272 |
| PetChim3 | 26.72 | 418 | 272 |
| PetChim4 | 26.727 | 418 | 272 |

TABLE A8
Formed NR2 products in bioconversions of
naringenin with different production strains

| strain | NR2 retention time [min] HPLC | ESI-MS | ESI-MSMS |
|---|---|---|---|
| PetC | 35.48 | 418 | 272 |
| PetD | 35.547 | 418 | 272 |
| PetF | 35.26 | 418 | 272 |
| PetS | 35.28 | 418 | 272 |
| PetChim1fs | 35.080 | 418 | 272 |
| PetChim3 | 35.267 | 418 | 272 |
| PetChim4 | 35.267 | 418 | 272 |

Biotransformation of the Flavanone Homoeriodictyol (HED) Using PetC

In preparative scale HED (5,7-Dihydroxy-2-(4-hydroxy-3-methoxyphenyl)-4-chromanone, CAS No. 446-71-9) was glycosylated by PetC. The biotransformation was performed following general preparative shake flask growth and bioconversion conditions.

The bioconversion of HED was monitored by HPLC analyses. The culture supernatant was loaded directly via pump flow to a preparative RP18 column (Agilent, USA). Stepwise elution was performed and nine fractions were collected according to table A5.

All nine fractions subsequently were analyzed by HPLC and ESI-TOF MS analyses. MS analyses of fractions 5 and 8 in negative ion mode showed that both contained a compound with the molecular weight of 448 Da which corresponded to the size of a HED-O-rhamnoside and were designated HEDR1 and HEDR3. MS analysis of fraction 7 (HEDR2) gave a molecular weight of 434 Da. However, ESI MS/MS analyses of all three fractions identified a leaving group of 146 Da suggesting a rhamnosidic residue also in fraction 7.

After HPLC polishing by a (PFP) phase and subsequent lyophilization the molecular structure of HEDR1 was solved by NMR analysis (Example B-1). HEDR1 (RT 28.26 min in analytical HPLC) was identified as the pure compound HED-5-O-α-L-rhamnoside.

TABLE A9
Fractionation of HED bioconversion by prepLC separation

| Frac | Well | | Volume | BeginTime | EndTime | Description | |
|---|---|---|---|---|---|---|---|
| # | # | Location | [μl] | [min] | [min] | [compound] | ESI-MS |
| 1 1 | Vial | 201 | 22503.75 | 5.0999 | 6.3501 Time | | |
| 2 1 | Vial | 202 | 28593.75 | 6.4115 | 8.0001 Time | | |
| 3 1 | Vial | 203 | 34927.50 | 8.0597 | 10.0001 Time | | |
| 4 1 | Vial | 204 | 20141.25 | 10.0611 | 11.1801 Time | | |
| 5 1 | Vial | 205 | 13695.00 | 11.2392 | 12.0001 Time | HEDR1 | 448 |
| 6 1 | Vial | 206 | 34931.25 | 12.0594 | 14.0001 Time | | |
| 7 1 | Vial | 207 | 25203.75 | 15.5999 | 17.0001 Time | HEDR2 | 434 |
| 8 1 | Vial | 208 | 38246.25 | 17.0753 | 19.2001 Time | HEDR3 | 448 |
| 9 1 | Vial | 209 | 66603.75 | 19.2999 | 23.0001 Time | HED | 302 |

Biotransformation Reactions Using PetC of the Isoflavone Genistein Using PetC

In preparative scale genistein (4′,5,7-Trihydroxyisoflavone, 5,7-dihydroxy-3-(4-hydrooxyphenyl)chromen-4-one, CAS No. 446-72-0) was glycosylated in bioconversion reactions using PetC. The biotransformation was performed in PBS following general preparative shake flask growth and bioconversion conditions.

The bioconversion of genistein was monitored by HPLC analyses. The genistein aglycon showed a RT of approx. 41 min. With reaction progress four peaks of reaction products (GR1-4) with RTs of approx. 26 min, 30 min, 34.7 min, and 35.6 min accumulated in the bioconversion (table A10).

The reaction was stopped by cell harvest after 40 h and in preparative RP18 HPLC stepwise elution was performed. All fractions were analyzed by HPLC and ESI-Q-TOF MS analyses. Fractions 3, 4, and 5, respectively, showed the molecular masses of genistein rhamnosides in MS analyses. Fraction 3 consisted of two separated major peaks (RT 26 min and 30 min). Fraction 4 showed a double peak of 34.7 min and 35.6 min, fraction 5 only the latter product peak at RT 35.6 min. Separate MS analyses of the peaks in negative ion mode revealed that all peaks contained compounds with the identical molecular masses of 416 which corresponded to the size of genistein-O-rhamnosides. NMR analysis of GR1 identified genistein-5,7-di-O-α-L-rhamnoside (Example B-9).

Biotransformation of the Isoflavone Biochanin a Using PetC

In preparative scale biochanin A (5,7-dihydroxy-3-(4-methoxyphenyl)chromen-4-one, CAS No. 491-80-5) was glycosylated in bioconversion reactions using PetC. The biotransformation was performed following general preparative shake flask growth and bioconversion conditions.

The bioconversion of biochanin A was monitored by HPLC. The biochanin A aglycon showed a RT of approx. 53.7 min. With reaction progress three product peaks at approx. 32.5′, 36.6′, and 45.6′ accumulated in the bioconversion (table A10). These were termed BR1, BR2, and BR3, respectively. The reaction was stopped by cell harvest after 24 h through centrifugation (13,000 g, 4° C.). The filtered supernatant was loaded to a preparative RP18 column and fractionated by stepwise elution. All fractions were analyzed by HPLC and ESI-Q-TOF MS analyses.

The PetC product BR1 with a RT of 32.5 min was identified by NMR as the 5,7-di-O-α-L-rhamnoside of biochanin A (Example B-4). NMR analysis of BR2 (RT 36.6′) gave the 5-O-α-L-rhamnoside (example B-5). In accordance to 5-O-α-L-rhamnosides of other flavonoids, e.g. HED-5-O-α-L-rhamnoside, BR2 was the most hydrophilic mono-rhamnoside with a slight retardation compared to HEDR1. Taking into account the higher hydrophobicity of the precursor biochanin A (RT 53.5′) due to less hydroxy groups and its C4′-methoxy function in comparison to a C4′-OH of genistein (RT 41′) the retard of BR2 compared to GR2 could be explained.

Biotransformation of the Flavone Chrysin Using PetC

In preparative scale chrysin (5,7-Dihydroxyflavone, 5,7-Dihydroxy-2-phenyl-4-chromen-4-one, CAS No. 480-40-0) was glycosylated in bioconversion reactions using PetC. The biotransformation was performed following stated preparative shake flask conditions in PBS.

The bioconversion of chrysin was monitored by HPLC analyses. The chrysin aglycon showed a RT of 53.5 min. In PetC bioconversions three reaction product peaks accumulated in the reaction, CR1 at RT 30.6 min, CR2 at RT36.4 min, and CR3 at RT43.4, respectively (table A10). All products were analyzed by HPLC and ESI-Q-TOF MS analyses.

CR1 was further identified by NMR as the 5,7-di-O-α-L-rhamnoside of chrysin (Example B-6) and in NMR analysis CR2 turned out to be the 5-O-α-L-rhamnoside (Example B-7). Like BR2, CR2 was also less hydrophilic than the 5-O-rhamnosides of flavonoids with free OH-groups at ring C, e.g. hesperetin and naringenin, although CR2 was the most hydrophilic mono-rhamnoside of chrysin.

Biotransformation of the Flavone Diosmetin Using PetC

Diosmetin (5,7-Trihydroxy-4′-methoxyflavone, 5,7-dihydroxy-2-(3-hydroxy-4-methoxyphenyl) chromen-4-one, CAS No. 520-34-3) was glycosylated in bioconversion reactions using PetC. The biotransformation was performed as stated before.

The bioconversion of diosmetin was monitored by HPLC. The diosmetin aglycon showed a RT of 41.5 min using the given method. With reaction progress three peaks of putative reaction products at 26.5′ (DR1), 29.1′ (DR2), and 36′ (DR3) accumulated (table A10).

The product DR2 with a RT of 29.1 min was further identified as the 5-O-α-L-rhamnoside of diosmetin (D5R) (Example B-10). DR1 was shown by ESI-MS analysis to be a di-rhamnoside of diosmetin. In accordance with the 5-O-α-L-rhamnosides of other flavonoids, e.g. hesperetin, DR2 had a similar retention in analytical RP18 HPLC-conditions.

Table A10 summarizes all reaction products of PetC biotransformations with the variety of flavonoid precursors tested.

Part B: NMR-Analyses of the Rhamnosylated Flavonoids

The following Examples were prepared according to the procedure described above in Part A.

### Example B-1: HED-5-O-α-L-rhamnoside

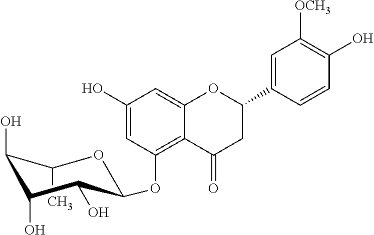

¹H NMR ((600 MHz Methanol-d₄): δ=7.06 (d, J=2.0 Hz, 1H), 7.05 (d, J=2.1 Hz, 1H), 6.91 (dt, J=8.2, 2.1, 0.4 Hz, 1H), 6.90 (ddd, J=8.1, 2.0, 0.6 Hz, 1H), 6.81 (d, J=8.1 Hz, 1H), 6.80 (d, J=8.1 Hz, 1H), 6.32 (d, J=2.3 Hz, 1H), 6.29 (d, J=2.3 Hz, 1H), 6.09 (t, J=2.3 Hz, 2H), 5.44 (d, J=1.9 Hz, 1H), 5.40 (d, J=1.9 Hz, 1H), 5.33 (dd, J=7.7, 2.9 Hz, 1H), 5.31 (dd, J=8.1, 3.0 Hz, 1H), 4.12 (ddd, J=11.2, 3.5, 1.9 Hz, 2H), 4.08 (dd, J=9.5, 3.5 Hz, 1H), 4.05 (dd, J=9.5, 3.5 Hz, 1H), 3.87 (s, 3H), 3.87 (s, 3H), 3.69-3.60 (m, 2H), 3.46 (td, J=9.5, 5.8 Hz, 2H), 3.06-3.02 (m, 1H), 3.02-2.98 (m, 1H), 2.64 (ddd, J=16.6, 15.5, 3.0 Hz, 2H), 1.25 (d, J=6.2 Hz, 3H), 1.23 (d, J=6.3 Hz, 3H).

### Example B-2: Hesperetin-5-O-α-L-rhamnoside

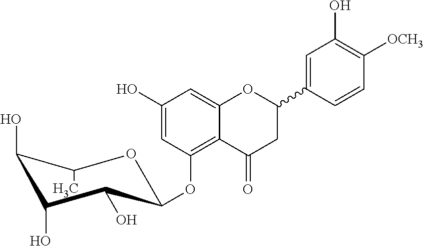

¹H-NMR (400 MHz, DMSO-d₆): δ=1.10 (3H, d, J=6.26 Hz, CH3), 2.45 (m, H-3(a), superimposed by DMSO), 2.97 (1H, dd, J=12.5, 16.5 Hz, H3(b)), 3.27 (1H, t, 9.49 Hz, H(b)), 3.48 (m, H(a), superimposed by HDO), 3.76 (3H, s, OCH3), 3.9-3.8 (2H, m, H(c), Hd), 5.31 (1H, d, 1.76 Hz, He), 5.33 (1H, dd, 12.5, 2.83 Hz, H2), 6.03 (1H, d, 2.19 Hz, H6/H8), 6.20 (1H, d, 2.19 Hz, H6/H8), 6.86 (1H, dd, 8.2, 2.0 Hz, H6′), 6.90 (1H, d, 2.0 Hz, H2′), 6.93 (1H, d, 8.2 Hz, H5′)

### Example B-3: Naringenin-5-O-α-L-rhamnoside

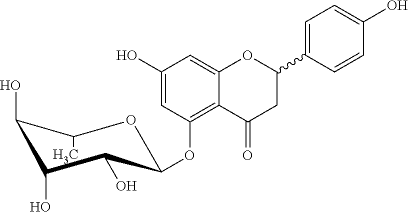

¹H NMR (600 MHz, DMSO-d6): δ=7.30 (d, J=6.9 Hz, 2H), 7.29 (d, J=6.9 Hz, 2H), 6.79 (d, J=8.6 Hz, 2H), 6.78 (d, J=8.6 Hz, 2H), 6.22 (d, J=2.3 Hz, 1H), 6.20 (d, J=2.2 Hz, 1H), 6.02 (d, J=2.2 Hz, 1H), 6.01 (d, J=2.2 Hz, 1H), 5.38 (dd, J=12.7, 3.1 Hz, 1H), 5.35 (dd, J=13.0, 2.5 Hz, 1H), 5.31 (d, J=1.8 Hz, 1H), 5.27 (d, J=1.9 Hz, 1H), 3.90-3.88 (m, 1H), 3.88-3.85 (m, 1H), 3.85-3.80 (m, 2H), 3.50 (dq, J=9.2, 6.2 Hz, 1H), 3.48 (dq, J=9.1, 6.2 Hz, 1H), 3.29 (t, J=9.8 Hz, 2H), 3.07-2.98 (m, 2H), 2.55-2.48 (m, 2H), 1.12 (d, J=6.2 Hz, 3H), 1.10 (d, J=6.2 Hz, 3H).

¹³C NMR (151 MHz, DMSO-d6): δ=187.75, 187.71, 164.04, 163.92, 163.80, 158.33, 158.23, 157.48, 157.44, 129.26, 129.24, 129.18, 129.15, 128.07, 128.00, 115.00, 105.19, 105.06, 98.58, 98.44, 97.25, 96.85, 96.77, 96.64, 78.03, 77.97, 71.67, 71.65, 69.98, 69.95, 69.66, 69.64, 44.78, 44.74, 17.80, 17.75.

### Example B-4: Biochanin A-5,7-di-O-α-L-rhamnoside

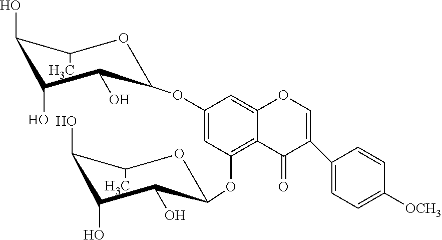

¹H NMR (400 MHz DMSO-d₆): δ=8.21 (s, 1H), 7.43 (d, J=8.5 Hz, 2H), 6.97 (d, J=8.6 Hz, 2H), 6.86 (d, J=1.8 Hz, 1H), 6.74 (d, J=1.8 Hz, 1H), 5.53 (d, J=1.6 Hz, 1H), 5.41 (d, J=1.6 Hz, 1H), 5.15 (s, 1H), 5.00 (s, 1H), 4.93 (s, 1H), 4.83 (s, 1H), 4.70 (s, 1H), 3.93 (br, 1H), 3.87 (br, 1H), 3.85 (br, 1H), 3.77 (s, 3H), 3.64 (dd, J=9.3, 3.0 Hz, 1H), 3.54 (dq, J=9.4, 6.4 Hz, 1H), 3.44 (dq, J=9.4, 6.4 Hz, 1H), 3.34 (br, 1H), 1.13 (d, J=6.1 Hz, 3H), 1.09 (d, J=6.1 Hz, 3H)

### Example B-5: Biochanin A 5-O-α-L-rhamnoside

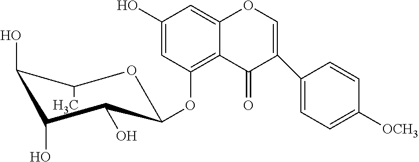

¹H NMR (400 MHz DMSO-d₆): δ=8.21 (s, 1H), 7.42 (d, J=8.7 Hz, 2H), 6.96 (d, J=8.7 Hz 2H), 6.55 (d, J=1.9 Hz, 1H), 6.48 (d, J=1.9 Hz, 1H), 5.33 (d, J=1.7 Hz, 1H), 5.1-4.1 (br, nH), 3.91 (br, 1H), 3.86 (d, J=9.7, 1H), 3.77 (s, 3H), 3.48 (br, superimposed by impurity, 1H), 3.44 (impurity), 3.3 (superimposed by HDO), 1.10 (d, J=6.2 Hz, 3H)

### Example B-6: Chrysin-di-5,7-O-α-L-rhamnoside

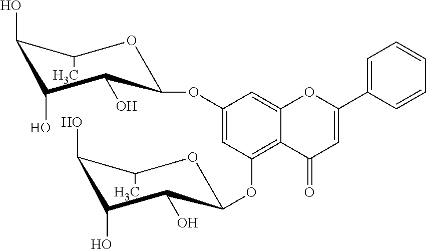

¹H NMR (400 MHz DMSO-d₆): δ=8.05 (m, 2H), 7.57 (m, 3H), 7.08 (s, 1H), 6.76 (d, J=2.3 Hz, 1H), 6.75 (s, 1H), 5.56 (d, J=1.6 Hz, 1H), 5.42 (d, J=1.6 Hz, 1H), 5.17 (s, 1H), 5.02 (s, 1H), 4.94 (s, 1H), 4.86 (s, 1H), 4.71 (s, 1H), 3.97 (br, 1H), 3.88 (dd, J=9.5, 3.1 Hz, 1H), 3.87 (br, 1H), 3.66 (dd, J=9.3, 3.4 Hz, 1H), 3.56 (dq, J=9.4, 6.2 Hz, 1H), 3.47 (dq, J=9.4, 6.2 Hz, 1H), 3.32 (superimposed by HDO, 2H), 1.14 (d, J=6.2 Hz, 3H), 1.11 (d, J=6.2 Hz, 3H)

### Example B-7: Chrysin-5-O-α-L-rhamnoside

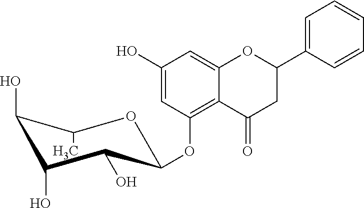

¹H NMR (400 MHz DMSO-d₆): δ=8.01 (m, 2H), 7.56 (m, 3H), 6.66 (s, 1H), 6.64 (d, J=2.1 Hz, 1H), 6.55 (d, J=2.1 Hz, 1H), 5.33 (d, J=1.5 Hz, 1H), 5.01 (s, 1H), 4.85 (d, J=4.7 Hz, 1H), 4.69 (s, 1H), 3.96 (br, 1H), 3.87 (md, J=8.2 Hz, 1H), 3.54 (dq, J=9.4, 6.2 Hz, 1H), 3.3 (superimposed by HDO), 1.11 (d, J=6.1 Hz, 3H)

### Example B-8: Silibinin-5-O-α-L-rhamnoside

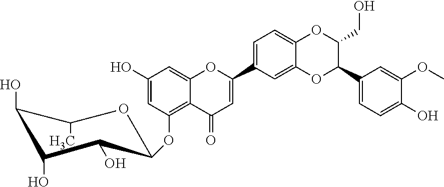

¹H NMR (400 MHz DMSO-d₆): δ=7.05 (dd, J=5.3, 1.9 Hz, 1H), 7.01 (br, 1H), 6.99 (ddd, J=8.5, 4.4, 1.8 Hz, 1H), 6.96 (dd, J=8.3, 2.3 Hz, 1H), 6.86 (dd, J=8.0, 1.8 Hz, 1H), 6.80 (d, J=8.0 Hz, 1H), 6.25 (d, J=1.9 Hz, 1H), 5.97 (dd, J=2.1, 3.7 Hz, 1H), 5.32 (d, J=1.6 Hz, 1H), 5.01 (d, J=11.2 Hz, 1H), 4.90 (d, J=7.3 Hz, 1H), 4.36 (ddd, J=11.2, 6.5, 4.6 Hz, 1H), 4.16 (ddd, J=7.6, 3.0, 4.6 Hz, 1H), 3.89 (m, 1H), 3.83 (br, 1H), 3.77 (d, J=1.8 Hz, 1H), 3.53 (m, 3H), 3.30 (superimposed by HDO, 3H), 1.13 (d, J=6.2 Hz, 3H)

### Example B-9: Genistein-5,7-di-O-α-L-rhamnoside

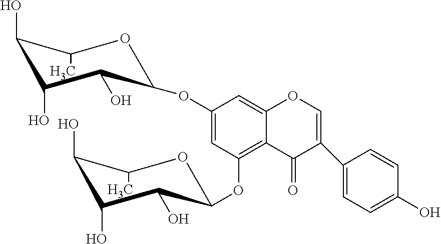

¹H NMR (400 MHz DMSO-d₆): δ=8.16 (s, 1H), 7.31 (d, J=8.4 Hz, 2H), 6.85 (d, J=2.1 Hz, 1H), 6.79 (d, J=8.4 Hz, 2H), 6.73 (d, J=2.1 Hz, 1H), 5.52 (d, J=1.8 Hz, 1H), 5.40 (d, J=1.8 Hz, 1H), 5.14 (d, J=3.8 Hz, 1H), 4.99 (d, J=3.8 Hz, 1H), 4.92 (d, J=5.2 Hz, 1H), 5.83 (d, J=5.2 Hz, 1H), 5.79 (d, J=5.5 Hz, 1H), 4.69 (d, J=5.5 Hz, 1H), 3.93 (br, 1H), 3.87 (br, 1H), 3.85 (br, 1H), 3.64 (br, 1H), 3.44 (m, 2H), 3.2 (superimposed by HDO, 2H), 1.12 (d, J=6.2 Hz, 3H), 1.09 (d, J=6.2 Hz, 3H)

### Example B-10: Diosmetin-5-O-α-L-rhamnoside

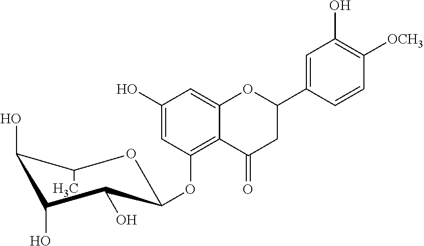

¹H NMR (600 MHz DMSO-d₆): δ=7.45 (dd, J=8.5, 2.3 Hz, 1H), 7.36 (d, J=2.3 Hz, 1H), 7.06 (d, J=8.6 Hz, 1H), 6.61 (d, J=2.3 Hz, 1H), 6.54 (d, J=2.3 Hz, 1H), 6.45 (s, 1H), 5.32 (d, J=1.7 Hz, 1H), 3.96 (dd, J=3.5, 2.0 Hz, 1H), 3.86 (m, 1H), 3.85 (s, 3H), 3.54 (dq, J=9.4, 6.3 Hz, 1H), 3.30 (superimposed by HDO, 1H), 1.11 (d, J=6.2, 3H)

Part C: Solubility

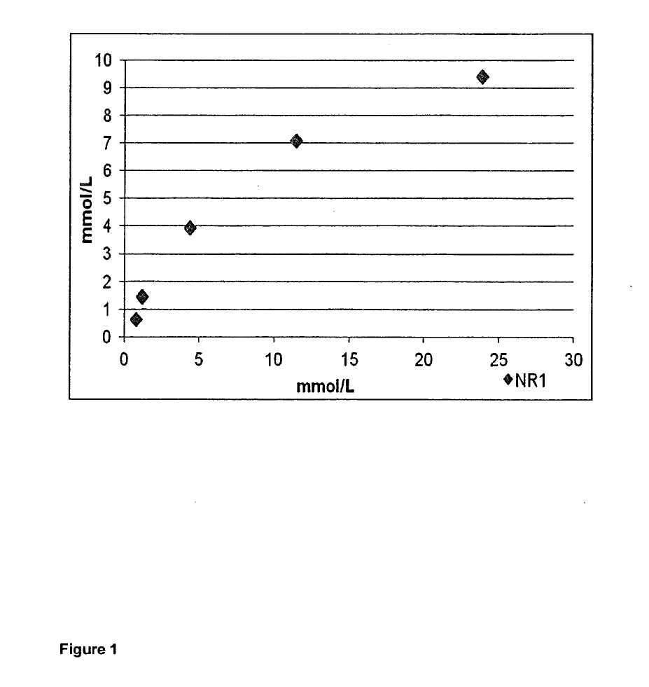

FIG. 1 illustrates the amounts of Naringenin-5-rhamnoside recaptured from a RP18 HPLC-column after loading of a 0.2 μm filtered solution containing defined amounts up to 25 mM of the same. Amounts were calculated from a regression curve. The maximum water solubility of Naringenin-5-rhamnoside approximately is 10 mmol/L, which is equivalent to 4.2 g/L.

The hydrophilicity of molecules is also reflected in the retention times in a reverse phase (RP) chromatography. Hydrophobic molecules have later retention times, which can be used as qualitative determination of their water solubility.

HPLC-chromatography was performed using a VWR Hitachi LaChrom Elite device equipped with diode array detection under the following conditions:

Column: Agilent Zorbax SB-C18 250×4.6 mm, 5 μM, Flow 1 mL/min

Mobile phases: A: H₂O+0.1% Trifluoro acetic acid (TFA);

B: ACN+0.1% TFA

Sample injection volume: 500 μL;

Gradient: 0-5 min: 5% B, 5-15 min: 15% B, 15-25 min: 25% B, 25-25 min: 35% B, 35-45 min: 40%, 45-55 min: 100% B, 55-63 min: 5% B

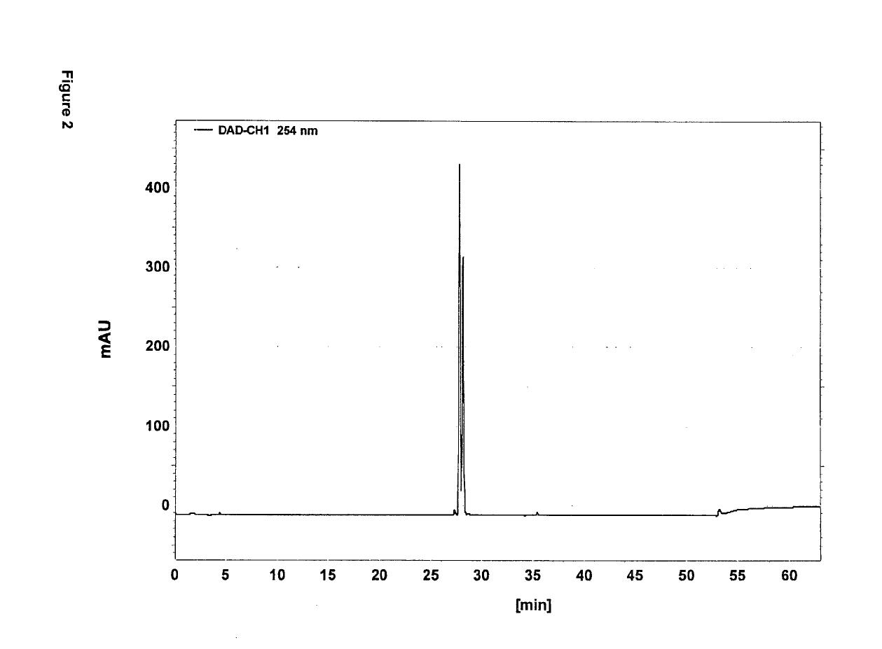

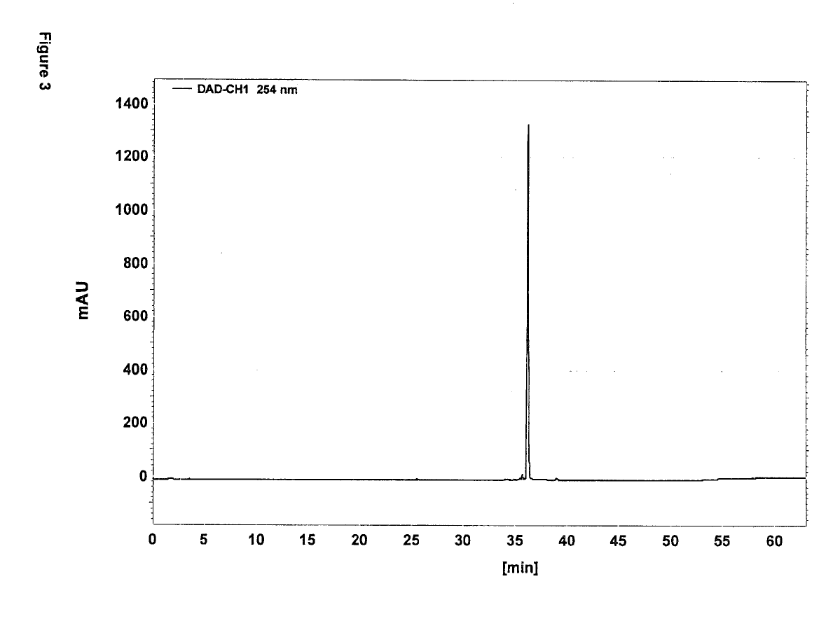

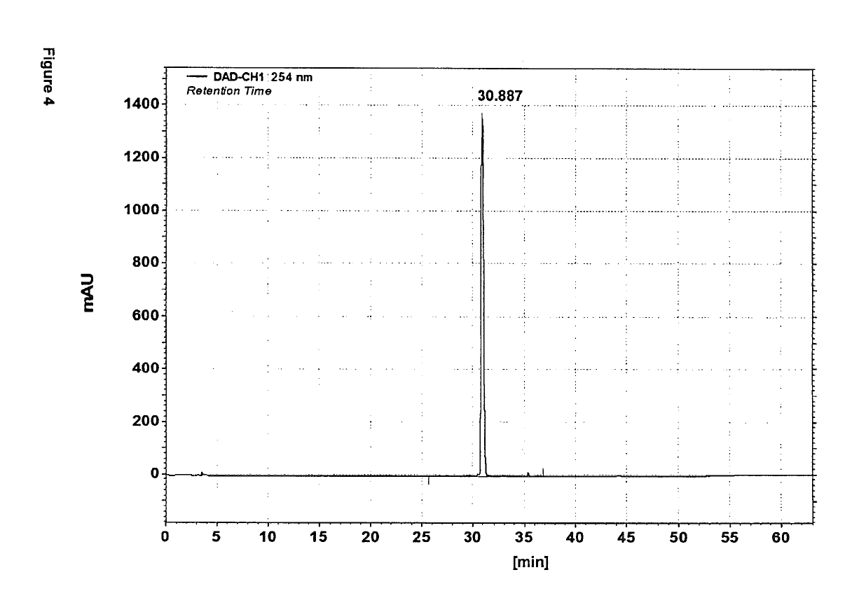

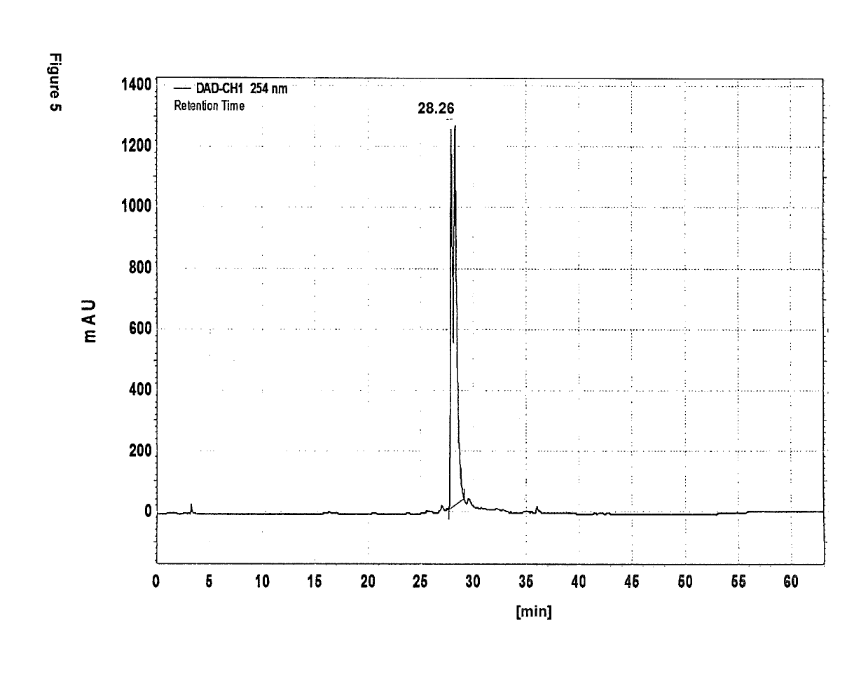

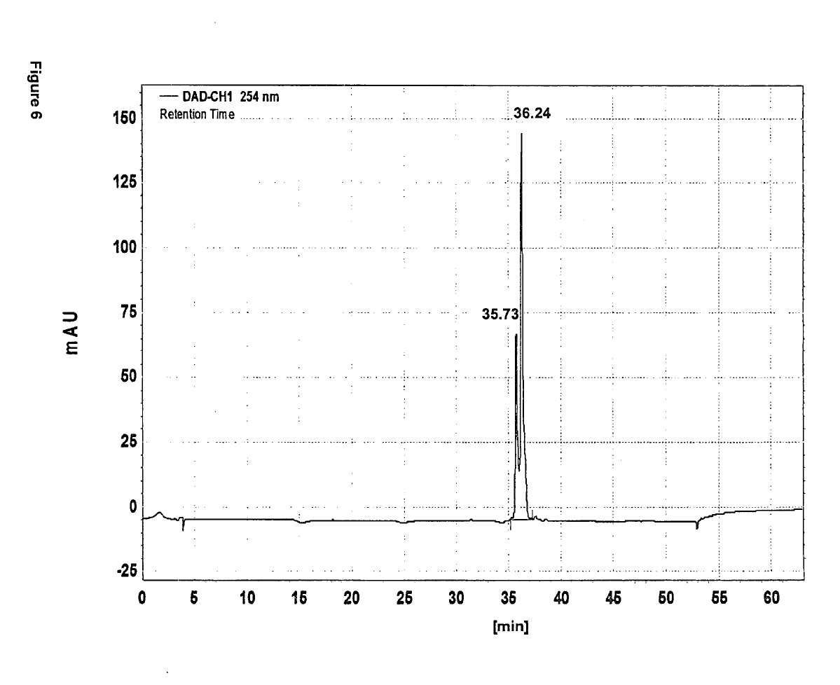

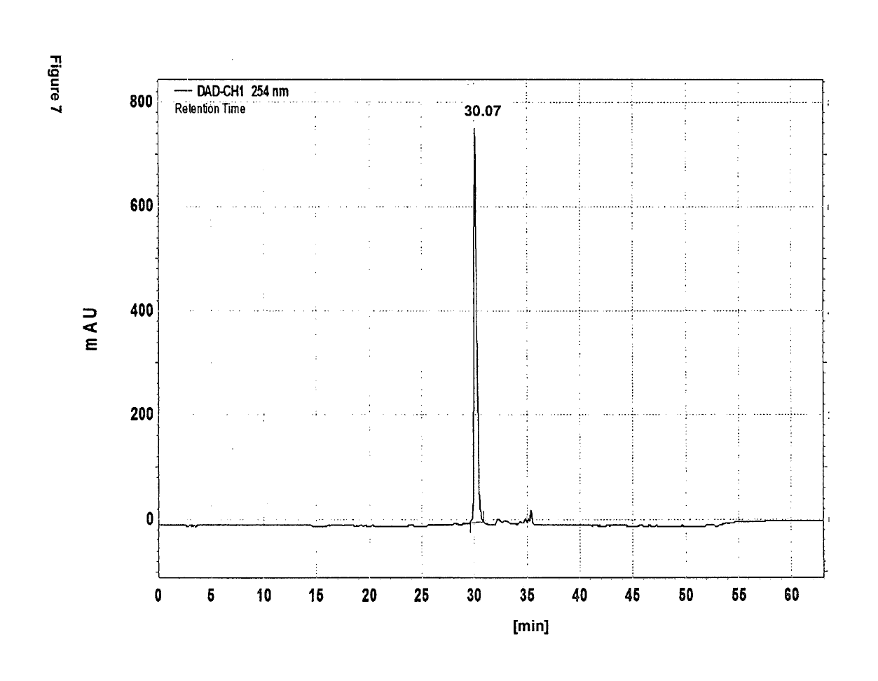

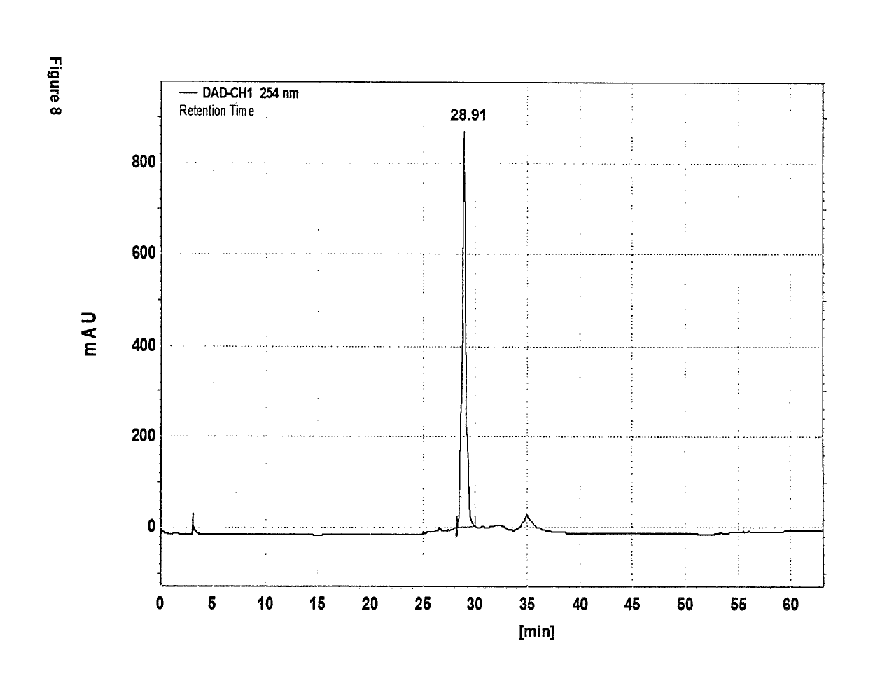

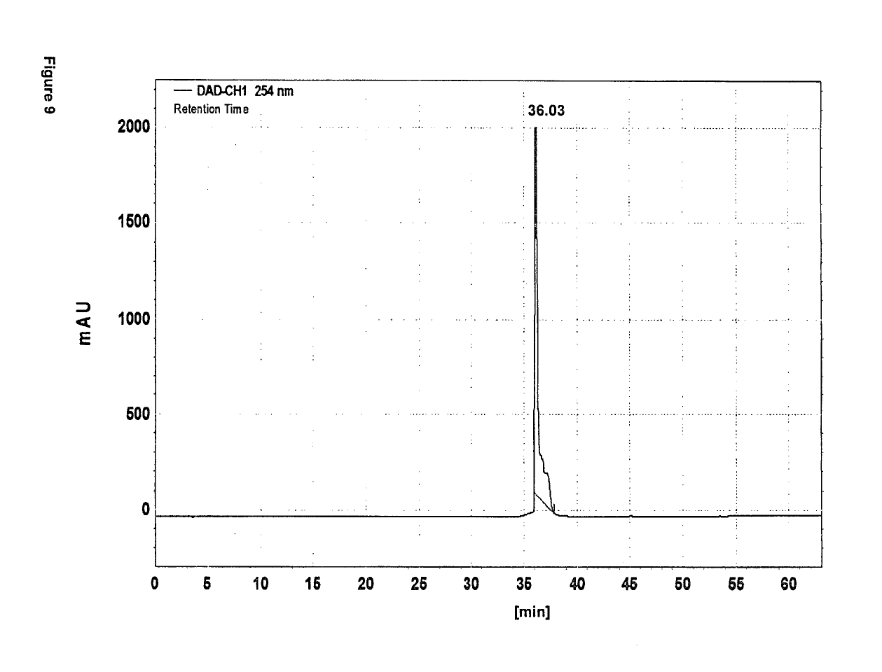

Table B1 contains a summary of the retention times according to FIGS. 2-9 and Example A-2.

TABLE B1
Retention times of flavonoid rhamnosides according to their linkage
position in analytical HPLC conditions given above

| | | | |
|---|---|---|---|
| Order of elution | N-5-O-α-L- | N-7-O-β-D- | N-4′-O-α-L- |
| | rhamnoside | glucoside | rhamnoside |
| Retention time | 27.3 | 30.9 | 36 |
| [min] | | | |
| Order of elution | HED-5-O-α-L- | HED-4′-O-β-D- | HEDR3 |
| | rhamnoside | glucoside | |
| Retention time | 28.3 | 30.1 | 35.8 |
| [min] | | | |
| Order of elution | HES-5-O-α-L- | HESR2 | HES-7-O-β-D- |
| | rhamnoside | | glucoside |
| Retention time | 28.9 | 36 | 31 |
| [min] | | | |

Generally, it is well known that glucosides of lipophilic small molecules in comparison to their corresponding rhamnosides are better water soluble, e.g. isoquercitrin (quercetin-3-glucoside) vs. quercitrin (quercetin-3-rhamnosides). Table B1 comprehensively shows the 5-O-α-L-rhamnosides are more soluble than α-L-rhamnosides and β-D-glucosides at other positions of the flavonoid backbone. All the 5-O-α-L-rhamnosides eluted below 30 min in RP18 reverse phase HPLC. In contrast, flavanone glucosides entirely were retained at RTs above 30 min independent of the position at the backbone. In case of HED it was shown that among other positions, here C4′ and C7, the differences concerning the retention times of the α-L-rhamnosides were marginal, whereas the C5 position had a strong effect on it. This was an absolutely unexpected finding.

The apparent differences of the solubility are clearly induced by the attachment site of the sugar at the polyphenolic scaffold. In 4-on-5-hydroxy benzopyranes the OH-group and the keto-function can form a hydrogen bond. This binding is impaired by the substitution of an α-L-rhamnoside at C5 resulting in an optimized solvation shell surrounding the molecule. Further, in aqueous environments the hydrophilic rhamnose residue at the C5 position might shield a larger area of the hydrophobic polyphenol with the effect of a reduced contact to the surrounding water molecules. Another explanation would be that the occupation of the C5 position more effectively forms a molecule with a spatial separation a hydrophilic saccharide part and a hydrophobic polyphenolic part. This would result in emulsifying properties and the formation of micelles. An emulsion therefore enhances the solubility of the involved compound.

Part D: Activity of Rhamnosylated Flavonoids

### Example D-1: Cytotoxicity of Flavonoid-5-O-α-L-rhamnosides

To determine the cytotoxicity of flavonoid-5-O-α-L-rhamnosides tests were performed versus their aglycon derivatives in cell monolayer cultures. For this purpose concentrations ranging from 1 μM to 250 μM were chosen. The viability of normal human epidermal keratinocytes (NHEK) was twice evaluated by a MTT reduction assay and morphological observation with a microscope. NHEK were grown at 37° C. and 5% CO₂ aeration in Keratinocyte-SFM medium supplemented with epidermal growth factor (EGF) at 0.25 ng/mL, pituitary extract (PE) at 25 μg/mL and gentamycin (25 μg/mL) for 24 h and were used at the 3rd passage. For cytotoxicity testing, pre-incubated NHEK were given fresh culture medium containing a specific concentration of test compound and incubated for 24 h. After a medium change at same test concentration cells were incubated a further 24 h until viability was determined. Test results are given in Table B2 and illustrated in FIG. 10.

TABLE B2
Cytotoxicity of flavonoid-5-O-α-L-rhamnosides
on normal human epidermal keratinocytes

| | | | [μM] from stock solution at 100 mM in DMSO | | | | | | | |
|---|---|---|---|---|---|---|---|---|---|---|
| Compound | Control | | 1 | 2.5 | 5 | 10 | 25 | 50 | 100 | 250 |
| Hesperetin | | | | | | | | | | |
| Viability (%) | 98 | 98 | 103 | 98 | 107 | 101 | 106 | 106 | 98 | 54 |
| | 102 | 102 | 106 | 109 | 106 | 105 | 109 | 106 | 100 | 59 |
| Mean | 100 | | 105 | 103 | 106 | 103 | 108 | 106 | 99 | 57 |
| sd | 2 | | 2 | 8 | 1 | 3 | 2 | 0 | 1 | 4 |
| Morph. obs. | + | | + | + | + | + | + | + | +/− | +/− |
| Hes-5-Rha | | | | | | | | | | |
| Viability (%) | 95 | 85 | 86 | 87 | 81 | 86 | 89 | 81 | 86 | 91 |
| | 118 | 103 | 108 | 113 | 95 | 103 | 112 | 93 | 108 | 102 |
| Mean | 100 | | 97 | 100 | 88 | 95 | 101 | 87 | 97 | 96 |
| sd | 14 | | 16 | 19 | 10 | 13 | 16 | 9 | 16 | 8 |
| Morph. obs. | + | | + | + | + | + | + | + | + | + |
| Naringenin | | | | | | | | | | |
| Viability (%) | 95 | 96 | 96 | 95 | 93 | 95 | 89 | 85 | 76 | 48 |
| | 104 | 105 | 95 | 92 | 91 | 95 | 94 | 94 | 74 | 47 |
| Mean | 100 | | 95 | 93 | 92 | 95 | 92 | 89 | 75 | 47 |
| sd | 5 | | 1 | 2 | 1 | 0 | 4 | 6 | 2 | 1 |
| Morph. obs. | + | | + | + | + | + | + | + | +/−, * | +/−, * |
| Nar-5-Rha | | | | | | | | | | |
| Viability (%) | 96 | 99 | 91 | 92 | 85 | 94 | 92 | 78 | 82 | 79 |
| | 101 | 104 | 111 | 93 | 88 | 100 | 98 | 91 | 90 | 87 |
| Mean | 100 | | 101 | 93 | 86 | 97 | 95 | 84 | 86 | 83 |
| sd | 3 | | 14 | 1 | 2 | 4 | 4 | 9 | 6 | 6 |
| Morph. obs. | + | | + | + | + | + | + | + | + | +/− |

Cytotoxicity measurements on monolayer cultures of NHEK demonstrated a better compatibility of the 5-O-α-L-rhamnosides versus their flavonoid aglycons at elevated concentration. Up to 100 μM no consistent differences were observed (FIG. 10). However, at 250 μM concentration of the aglycons hesperetin and naringenin the viability of NHEK was decreased to about 50% while the mitochondrial activity of NHEK treated with the corresponding 5-O-α-L-rhamnosides was still unaffected compared to lower concentrations. In particular these results were unexpected as the solubility of flavonoid aglycons generally is below 100 μM in aqueous phases while that of glycosidic derivatives is above 250 μM. These data clearly demonstrated that the 5-O-α-L-rhamnosides were less toxic to the normal human keratinocytes.

### Example D-2: Anti-Inflammatory Properties

Anti-Inflammatory Potential

NHEK were pre-incubated for 24 h with the test compounds. The medium was replaced with the NHEK culture medium containing the inflammatory inducers (PMA or Poly I:C) and incubated for another 24 hours. Positive and negative controls ran in parallel. At the endpoint the culture supernatants were quantified of secreted IL-8, PGE2 and TNF-α by means of ELISA.

Anti-Inflammatory Effects of 5-O-rhamnosides in NHEK Cell Cultures

TABLE B3
Inhibition of 5-O-rhamnosides on Cytokine release in human keratinocytes (NHEK)

| | | | | | | | % stim. | | | | | |
|---|---|---|---|---|---|---|---|---|---|---|---|---|
| Compound | | | Cytokine [pg/mL] | | | | control | | | Inhibition | | |
| Conc. | | Stimulation | Type | | Mean | sd | % | sd | p⁽¹⁾ | % | sd | p⁽¹⁾ |
| Non- | Control | | | 96 | 126 | 18 | 8 | 1 | *** | 100 | 1 | *** |
| stimulat | | | | 157 | | | | | | | | |
| | | | | 127 | | | | | | | | |
| Stimulated | Control | | | 1846 | 1569 | 141 | 100 | 9 | — | 0 | 10 | — |
| conditions: | | | | 1480 | | | | | | | | |
| PMA - 1 μg/ml | | | | 1381 | | | | | | | | |
| | Indomethacin | | | 39 | 39 | 0 | 2 | 0 | *** | 106 | 0 | *** |
| | 10⁻⁶ M | | | 39 | | | | | | | | |
| | | | | 39 | | | | | | | | |
| | Dexamethasone | | | 1318 | 1437 | 168 | 92 | 11 | — | 9 | 12 | — |
| | 10⁻⁶ M | | | 1556 | | | | | | | | |
| | HESR1 | PMA | PGE₂ | 582 | 507 | 107 | 32 | 7 | — | 74 | 7 | — |
| | (HES-5- | | | 431 | | | | | | | | |
| | Rha) | | IL-8 | 3242 | 2843 | 564 | 98 | 19 | — | 34 | 17 | |
| | 100 μM | | | 2445 | | | | | | | | |
| | | poly(I:C) | IL-8 | 2617 | 2793 | 250 | 76 | 7 | | 24 | 7 | |
| | | | | 2970 | | | | | | | | |
| | | | TNFα | 416 | 423 | 9 | 75 | 2 | | 26 | 2 | |
| | | | | 429 | | | | | | | | |
| | NR1 | PMA | PGE₂ | 851 | 1271 | 594 | 81 | 38 | — | 21 | 41 | — |
| | (N-5- | | | 1691 | | | | | | | | |
| | Rha) | | IL-8 | 2572 | 2564 | 12 | 88 | 0 | — | 12 | 0 | — |
| | 100 μM | | | 2555 | | | | | | | | |
| | | poly(I:C) | IL-8 | 3055 | 3154 | 140 | 86 | 4 | | 14 | 4 | |
| | | | | 3253 | | | | | | | | |
| | | | TNFα | 516 | 516 | 0 | 92 | 0 | | 8 | 0 | |
| | | | | 516 | | | | | | | | |

Compared to control experiments the 5-O-rhamnosides showed anti-inflammatory activities on human keratinocytes concerning three different inflammation markers IL-8, TNFα, and PGE₂ under inflammatory stimuli (PMA, poly(I:C)). Especially, the activity of HESR1 on PGE₂ was remarkable with a 74% inhibition. An anti-inflammatory activity is well documented for flavonoid derivatives. And several authors reported their action via COX, NFκB, and MAPK pathways (Biesalski (2007) Curr Opin Clin Nutr Metab Care 10(6):724-728, Santangelo (2007) Ann Ist Super Sanita 43(4): 394-405). However, the exceptional water solubility of flavonoid-5-O-rhamnosides disclosed here allows much higher intracellular concentrations of these compounds than achievable with their rarely soluble aglycon counterparts. The aglycon solubilities are mostly below their effective concentration. Thus, the invention enables higher efficacy for anti-inflammatory purposes.

Among many other regulatory activities TNFα also is a potent inhibitor of hair follicle growth (Lim (2003) Korean J Dermatology 41: 445-450). Thus, TNFα inhibiting compounds contribute to maintain normal healthy hair growth or even stimulate it.

### Example D-3: Antioxidative Properties

Antioxidative Effects of 5-O-rhamnosides in NHEK Cell Cultures

Pre-incubated NHEK were incubated with the test compound for 24 h. Then the specific fluorescence probe for the measurement of hydrogen peroxide (DHR) or lipid peroxides (C11-fluor) was added and incubated for 45 min. Irradiation occurred with in H₂O₂ determination UVB at 180 mJ/cm² (+UVA at 2839 mJ/cm²) or UVB at 240 mJ/cm² (+UVA at 3538 mJ/cm²) in lipid peroxide, respectively, using a SOL500 Sun Simulator lamp. After irradiation the cells were post-incubated for 30 min before flow-cytometry analysis.

TABLE B4
Protection of 5-O-rhamnosides against UV-induced H₂O₂ stress in NHEK cells

| | | | | | | | % irradiated | | | | | |
|---|---|---|---|---|---|---|---|---|---|---|---|---|
| Test | | | | H₂O₂ (AU) | | | control | | | Protection | | |
| compound | | Concentration | | (DHR GMFI) | Mean | sd | % | sd | p⁽¹⁾ | % | sd | p⁽¹⁾ |
| Non-Irradiated | No DHR | — | | 9 | 8.77 | 0 | — | — | — | — | — | — |
| condition | probe | | | 8 | | | | | | | | |
| | | | | 9 | | | | | | | | |
| | Control | | | 311 | 316.33 | 3 | 17 | 0 | ** | 100 | 0 | ** |
| | | | | 319 | | | | | | | | |
| | | | | 319 | | | | | | | | |
| Irradiated | Control | | | 1770 | 1846.83 | 209 | 100 | 11 | — | 0 | 14 | — |
| conditions: | | | | 1307 | | | | | | | | |
| 180 mJ/cm² UVB | | | | 2388 | | | | | | | | |
| (2839 mJ/ cm² UVA) | | | | 1182 | | | | | | | | |
| | | | | 2169 | | | | | | | | |
| | | | | 2265 | | | | | | | | |
| | BHA | 100 | μM | 740 | 776 | 29 | 42 | 2 | * | 70 | 2 | * |
| | | | | 834 | | | | | | | | |
| | | | | 754 | | | | | | | | |
| | Vit. E | 50 | μM | 628 | 655 | 17 | 35 | 1 | ** | 78 | 1 | ** |
| | | | | 650 | | | | | | | | |
| | | | | 687 | | | | | | | | |
| | HESR1 | 100 | μM | 1046 | 1152 | 150 | 62 | 8 | — | 45 | 10 | |
| | | | | 1258 | | | | | | | | |
| | NR1 | 100 | μM | 2531 | 2516.5 | 21 | 136 | 1 | — | −44 | 1 | |
| | | | | 2502 | | | | | | | | |

TABLE B5
Protection of 5-O-rhamnosides against UV-induced lipid peroxide in NHEK cells

| | | | | | | | | % Irradiated | | | | | |
|---|---|---|---|---|---|---|---|---|---|---|---|---|---|
| Test | | | | C11-fluor(AU) | | | | control | | | Protection | | |
| compound | | Concentration | | GMFI | 1/GMFI | Mean | sd | % | sd | p⁽¹⁾ | % | sd | p⁽¹⁾ |
| Non-Irradiated | No C11- | — | | 3 | 3.1E−01 | 3.1E−01 | 1.1E−02 | — | — | — | — | — | — |
| condition | fluor | | | 3 | 3.0E−01 | | | | | | | | |
| | probe | | | 3 | 3.3E−01 | | | | | | | | |
| | Control | — | | 9049 | 1.1E−04 | 1.1E−04 | 7.6E−06 | 23 | 2 | *** | 100 | 2 | *** |
| | | | | 10874 | 9.2E−05 | | | | | | | | |
| | | | | 8504 | 1.2E−04 | | | | | | | | |
| Irradiated | Control | | | 2273 | 4.4E−04 | 4.6E−04 | 1.2E−05 | 100 | 3 | — | 0 | 3 | — |
| conditions: | | | | 2072 | 4.8E−04 | | | | | | | | |
| 240 mJ/cm² UVB | | | | 2166 | 4.6E−04 | | | | | | | | |
| (3538 mJ/cm² UVA) | BHT | 50 | μM | 3139 | 3.2E−04 | 3.3E−04 | 8.5E−06 | 72 | 2 | *** | 37 | 2 | *** |
| | | | | 3047 | 3.3E−04 | | | | | | | | |
| | | | | 2877 | 3.5E−04 | | | | | | | | |
| | HESR1 | 100 | μM | 1671 | 6.0E−04 | 6.4E−04 | 6.3E−05 | 99 | 10 | — | 1 | 12 | |
| | | | | 1455 | 6.9E−04 | | | | | | | | |
| | NR1 | 100 | μM | 2414 | 4.1E−04 | 4.3E−04 | 2.1E−05 | 93 | 4 | — | 9 | 6 | — |

| | |
|---|---|
| 2255 | 4.4E−04 |

An anti-oxidative function of the tested flavonoid-5-O-rhamnosides could be observed for HESR1 on mitochondrially produced hydrogen peroxids species and for NR1 on lipid peroxides, respectively. However, it is argued that these parameters are influenced also by different intracellular metabolites and factors, e.g. gluthation. Hence, interpretation of anti-oxidative response often is difficult to address to a single determinant.

### Example D-4: Stimulating Properties of 5-O-rhamnosides

Tests were performed with normal human dermal fibroblast cultures at the 8^{th} passage. Cells were grown in DMEM supplemented with glutamine at 2 mM, penicillin at 50 U/mL and streptomycin (50 μg/mL) and 10% of fetal calf serum (FCS) at 37° C. in a 5% CO₂ atmosphere.

Stimulation of Flavonoid-5-O-rhamnosides on Syntheses of Procollagen I, Release of VEGF, and Fibronectin Production in NHDF Cells

Fibroblasts were cultured for 24 hours before the cells were incubated with the test compounds for further 72 hours. After the incubation the culture supernatants were collected in order to measure the released quantities of procollagen I, VEGF, and fibronectin by means of ELISA. Reference test compounds were vitamin C (procollagen I), PMA (VEGF), and TGF-β (fibronectin).

TABLE B6
Stimulation of 5-O-rhamnosides on procollagen I synthesis in NHDF cells

| | | | Basic data | | | | | | | | |
|---|---|---|---|---|---|---|---|---|---|---|---|
| | | | Pro- | | | | | | Normalized data | | |
| Treatment | | | collagen I | | | % | | | % | | |
| Compound | Conc. | | (ng/ml) | Mean | sd | Control | sd | p⁽¹⁾ | Stimulation | sd | p⁽¹⁾ |
| Control | — | | 1893 | 1667 | 122 | 100 | 7 | — | 0 | 7 | — |
| | | | 1473 | | | | | | | | |
| | | | 1637 | | | | | | | | |
| Vitamin C | 20 | μg/ml | 4739 | 5272 | 323 | 316 | 19 | *** | 216 | 19 | *** |
| | | | 5854 | | | | | | | | |
| | | | 5225 | | | | | | | | |
| NR1 | 100 | μM | 1334 | 1097 | 335 | 66 | 20 | — | −34 | 20 | |
| | | | 860 | | | | | | | | |
| HESR1 | 100 | μM | 1929 | 1968 | 55 | 118 | 3 | — | 18 | 3 | |
| | | | 2007 | | | | | | | | |

TABLE B7
Stimulation of 5-O-rhamnosides on VEGF release in NHDF cells

| | | | Basic data | | | | | | | | |
|---|---|---|---|---|---|---|---|---|---|---|---|
| | | | | Mean | | | | | Normalized data | | |
| Treatment | | | VEGF | VEGF | | % | sd | | % | sd | |
| Compound | Conc. | | (pg/ml) | (pg/ml) | sd | Control | (%) | p⁽¹⁾ | Stimulation | (%) | p⁽¹⁾ |
| Control | — | | 83 | 72 | 6 | 100 | 9 | — | 0 | 9 | — |
| | | | 73 | | | | | | | | |
| | | | 61 | | | | | | | | |
| PMA | 1 | μg/ml | 150 | 148 | 3 | 205 | 4 | *** | 105 | 4 | *** |
| | | | 150 | | | | | | | | |
| | | | 143 | | | | | | | | |
| NR1 | 100 | μM | 90 | 92 | 3 | 128 | 4 | — | 28 | 4 | |
| | | | 94 | | | | | | | | |
| HESR1 | 100 | μM | 70 | 73 | 5 | 101 | 6 | — | 1 | 6 | |

76

TABLE B8
Stimulation of 5-O-rhamnosides on fibronectin synthesis in NHDF cells

| | | | Basic data | | | | | | Normalized data | | |
|---|---|---|---|---|---|---|---|---|---|---|---|
| Treatment | | | Fibronectin | Mean | | % | sd | | % | sd | |
| Compound | Conc. | | (ng/ml) | (ng/ml) | sd | Control | (%) | p⁽¹⁾ | Stimulation | (%) | p⁽¹⁾ |
| Control | — | | 6017 | 6108 | 86 | 100 | 1 | — | 0 | 1 | — |
| | | | 6281 | | | | | | | | |
| | | | 6027 | | | | | | | | |
| TGF-β | 10 | ng/ml | 10870 | #### | 95 | 181 | 2 | *** | 81 | 2 | *** |
| | | | 11178 | | | | | | | | |
| | | | 11128 | | | | | | | | |
| NR1 | 100 | μM | 6833 | 7326 | 698 | 120 | 11 | — | 20 | 11 | |
| | | | 7820 | | | | | | | | |
| HESR1 | 100 | μM | 5843 | 5853 | 14 | 96 | 0 | — | −4 | 0 | |
| | | | 5864 | | | | | | | | |

Results demonstrated that flavonoid-5-O-rhamnosides can positively affect extracellular matrix components. HESR1 stimulated procollagen I synthesis in NHDF by about 20% at 100 μM. NR1 at 100 μM had a stimulating effect on fibronectin synthesis with an increase of 20% in NHDF. Both polymers are well known to be important extracellular tissue stabilization factors in human skin. Hence substances promoting collagen synthesis or fibronectin synthesis support a firm skin, reduce wrinkles and diminish skin aging. VEGF release was also stimulated approx. 30% by NR1 indicating angiogenic properties of flavonoid-5-O-rhamnosides. Moderate elevation levels of VEGF are known to positively influence hair and skin nourishment through vascularization and thus promote e.g. hair growth (Yano (2001) J Clin Invest 107:409-417, KR101629503B1). Also, Fibronectin was described to be a promoting factor on human hair growth as stated in US 2011/0123481 A1. Hence, NR1 stimulates hair growth by stimulating the release of VEGF as well as the synthesis of fibronectin in normal human fibroblasts.

Stimulation of Flavonoid-5-O-rhamnosides on MMP-1 Release in UVA-Irradiated NHDF

Human fibroblasts were cultured for 24 hours before the cells were pre-incubated with the test or reference compounds (dexamethasone) for another 24 hours. The medium was replaced by the irradiation medium (EBSS, CaCl₂ 0.264 g/L, MgClSO₄ 0.2 g/L) containing the test compounds) and cells were irradiated with UVA (15 J/cm²). The irradiation medium was replaced by culture medium including again the test compounds incubated for 48 hours. After incubation the quantity of matrix metallopeptidase 1 (MMP-1) in the culture supernatant was measured using an ELISA kit.

TABLE B10
Stimulation of 5-O-rhamnosides on UV-induced MMP-1 release in NHDF cells

| | | | | Basic data | | | | | | | | |
|---|---|---|---|---|---|---|---|---|---|---|---|---|
| | | | | | Mean | | % | | | Normalized data | | |
| Treatment | | | | MMP-1 | MMP-1 | | Irradiated | sd | | % | sd | |
| Test compound | | Conc. | | (ng/ml) | (ng/ml) | sd | control | (%) | p⁽¹⁾ | Protection | (%) | p⁽¹⁾ |
| Non- | Control | — | | 28.1 | 25.5 | 1.6 | 36 | 2 | ** | 100 | 4 | ** |
| Irradiated | | | | 26.1 | | | | | | | | |
| | | | | 22.5 | | | | | | | | |
| Irradiated | Control | — | | 83.7 | 71.0 | 7.1 | 100 | 10 | — | 0 | 16 | — |
| conditions: | | | | 59.1 | | | | | | | | |
| 15 J/cm² UVA | | | | 70.3 | | | | | | | | |
| | Dexamethason | 10⁻⁷ | M | 2.5 | 2.9 | 0.2 | 4 | 0 | *** | 150 | 0 | *** |
| | | | | 3.1 | | | | | | | | |
| | | | | 3.2 | | | | | | | | |
| | NR1 | 100 | μM | 211.7 | 240.3 | 40.3 | 338 | 57 | — | −372 | 89 | |
| | | | | 268.8 | | | | | | | | |
| | HESR1 | 100 | μM | 87.0 | 82.2 | 6.8 | 116 | 10 | — | −25 | 15 | |
| | | | | 77.4 | | | | | | | | |

Flavonoid-5-O-rhamnosides showed high activities on MMP-1 levels in NHDF. NR1 caused a dramatic upregulation of MMP-1 biosynthesis nearly 4-fold in UV-irradiated conditions. MMP-1 also known as interstitial collagenase is responsible for collagen degradation in human tissues. Here, MMP-1 plays important roles in pathogenic arthritic diseases but was also correlated with cancer via metastasis and tumorigenesis (Vincenti (2002) Arthritis Res 4:157-164, Henckels (2013) F1000Research 2:229). Additionally, MMP-1 activity is important in early stages of wound healing (Caley (2015) Adv Wound Care 4: 225-234). Thus, MMP-1 regulating compounds can be useful in novel wound care therapies, especially if they possess anti-inflammatory and VEGF activities as stated above.

NR1 even enables novel therapies against arthritic diseases via novel biological regulatory targets. For example, MMP-1 expression is regulated via global MAPK or NFκB pathways (Vincenti and Brinckerhoff 2002, Arthritis Research 4(3):157-164). Since flavonoid-5-O-rhamnosides are disclosed here to possess anti-inflammatory activities and reduce IL-8, TNFα, and PGE-2 release, pathways that are also regulated by MAPK and NFκB. Thus, one could speculate that MMP-1 stimulation by flavonoid-5-O-rhamnosides is due to another, unknown pathway that might be addressed by novel pharmaceuticals to fight arthritic disease.

Current dermocosmetic concepts to reduce skin wrinkles address the activity of collagenase. Next to collagenase inhibition one contrary concept is to support its activity. In this concept misfolded collagene fibres that solidify wrinkles within the tissue are degraded by the action of collagenases. Simultaneously, new collagene has to be synthesized by the tissue to rebuild skin firmness. In this concept, the disclosed flavonoid-5-O-rhamnosides combine ideal activities as they show stimulating activity of procollagen and MMP-1.

Finally, MMP-1 upregulating flavonoid-5-O-rhamnosides serve as drugs in local therapeutics to fight abnormal collagene syndroms like Dupuytren's contracture.

### Example D-5: Modulation of Transcriptional Regulators by Flavonoid-5-O-rhamnosides

NF-κB Activity in Fibroblasts

NIH3T3-KBF-Luc cells were stably transfected with the KBF-Luc plasmid (Sancho (2003) Mol Pharmacol 63:429-438), which contains three copies of NF-κB binding site (from major histocompatibility complex promoter), fused to a minimal simian virus 40 promoter driving the luciferase gene. Cells (1×10⁴ for NIH3T3-KBF-Luc) were seeded the day before the assay on 96-well plate. Then the cells were treated with the test substances for 15 min and then stimulated with 30 ng/ml of TNFα. After 6 h, the cells were washed twice with PBS and lysed in 50 μl lysis buffer containing 25 mM Tris-phosphate (pH 7.8), 8 mM MgCl2, 1 mM DTT, 1% Triton X-100, and 7% glycerol during 15 min at RT in a horizontal shaker. Luciferase activity was measured using a GloMax 96 microplate luminometer (Promega) following the instructions of the luciferase assay kit (Promega, Madison, Wis., USA). The RLU was calculated and the results expressed as percentage of inhibition of NF-κB activity induced by TNFα (100% activation) (tables B10.1-B10.3). The experiments for each concentration of the test items were done in triplicate wells.

TABLE B10.1
Influence of 5-O-rhamnosides on NF-κB activity in NIH3T3 cells

| | | | | | | RLU | % |
|---|---|---|---|---|---|---|---|
| | | RLU 1 | RLU 2 | RLU 3 | MEAN | specific | Activation |
| | Control | 38240 | 38870 | 34680 | 37263 | 0 | 0 |
| | TNFα 30 ng/ml | 115870 | 120220 | 121040 | 119043 | 81780 | 100.0 |
| +30 ng/ml TNFα | HESR1 10 μM | 186120 | 181040 | 182280 | 183147 | 145883 | 178.4 |
| | HESR1 25 μM | 218940 | 216580 | 213320 | 216280 | 179017 | 218.9 |
| | NR1 10 μM | 134540 | 126580 | 130240 | 130453 | 93190 | 114.0 |
| | NR1 25 μM | 151080 | 151840 | 143870 | 148930 | 111667 | 136.5 |
| | Chrysin 10 μM | 301630 | 274240 | 303950 | 293273 | 256010 | 313.0 |
| | Chrysin 25 μM | 273410 | 272580 | 285980 | 277323 | 240060 | 293.5 |

TABLE B10.2
Influence of 5-O-rhamnosides on NF-κB activity in NIH3T3 cells

| | | | | | | RLU | % |
|---|---|---|---|---|---|---|---|
| | | RLU 1 | RLU 2 | RLU 3 | MEAN | specific | Activation |
| | Control | 23060 | 23330 | 23700 | 23363 | 0 | 0 |
| | TNFα 30 ng/ml | 144940 | 156140 | 160200 | 153760 | 130397 | 100.0 |
| +30 ng/ml TNFα | CR1 10 μM | 157870 | 169000 | 173010 | 166627 | 143263 | 109.9 |
| | CR1 25 μM | 175140 | 183630 | 183960 | 180910 | 157547 | 120.8 |
| | CR2 10 μM | 156600 | 160140 | 151070 | 155937 | 132573 | 101.7 |
| | CR2 25 μM | 170390 | 179220 | 163490 | 171033 | 147670 | 113.2 |
| | Diosmetin 10 μM | 398660 | 411390 | 412940 | 407663 | 384300 | 294.7 |
| | Diosmetin 25 μM | 448530 | 452660 | 451610 | 450933 | 427570 | 327.9 |
| | DR2 10 μM | 211150 | 215320 | 213260 | 213243 | 189880 | 145.6 |
| | DR2 25 μM | 245900 | 241550 | 234880 | 240777 | 217413 | 166.7 |
| | Biochanin A | 588070 | 586440 | 579220 | 584577 | 561213 | 430.4 |
| | 10 μM | | | | | | |
| | Biochanin A | 570360 | 573190 | 594510 | 579353 | 555990 | 426.4 |
| | 25 μM | | | | | | |
| | BR1 10 μM | 259120 | 247590 | 229500 | 245403 | 222040 | 170.3 |
| | BR1 25 μM | 211660 | 208010 | 203720 | 207797 | 184433 | 141.4 |
| | BR2 10 μM | 205410 | 202640 | 202940 | 203663 | 180300 | 138.3 |
| | BR2 25 μM | 237390 | 235850 | 235350 | 236197 | 212833 | 163.2 |

TABLE B10.3
Influence of 5-O-rhamnosides on NF-κB activity in NIH3T3 cells

| | | | | | | RLU | % |
|---|---|---|---|---|---|---|---|
| | | RLU 1 | RLU 2 | RLU 3 | MEAN | specific | Activation |
| | Control | 32200 | 33240 | 33100 | 32847 | 0 | 0 |
| | TNFα 30 ng/ml | 179150 | 179270 | 184270 | 180897 | 148050 | 100.0 |
| +30 ng/ml TNFα | Silibinin 10 μM | 249050 | 238550 | 231180 | 239593 | 206747 | 139.6 |
| | Silibinin 25 μM | 212420 | 210050 | 200660 | 207710 | 174863 | 118.1 |
| | SR1 10 μM | 269710 | 262180 | 254090 | 261993 | 229147 | 154.8 |
| | SR1 25 μM | 174940 | 171280 | 171730 | 172650 | 139803 | 94.4 |

It was reported that NF-κB activity is reduced by many flavonoids (Prasad (2010) Planta Med 76: 1044-1063). Chrysin was reported to inhibit NF-κB activity through the inhibition of IκBα phosphorylation (Romier (2008) Brit J Nutr 100: 542-551). However, when NIH3T3-KBF-Luc cells were stimulated with TNFα the activity of NF-κB was generally co-stimulated by flavonoids and their 5-O-rhamnosides at 10 μM and 25 μM, respectively.

STAT3 Activity

HeLa-STAT3-luc cells were stably transfected with the plasmid 4xM67 pTATA TK-Luc. Cells (20×10³ cells/ml) were seeded 96-well plate the day before the assay. Then the cells were treated with the test substances for 15 min and then stimulated with IFN-γ 25 IU/ml. After 6 h, the cells were washed twice with PBS and lysed in 50 μl lysis buffer containing 25 mM Tris-phosphate (pH 7.8), 8 mM MgCl₂, 1 mM DTT, 1% Triton X-100, and 7% glycerol during 15 min at RT in a horizontal shaker. Luciferase activity was measured using GloMax 96 microplate luminometer (Promega) following the instructions of the luciferase assay kit (Promega, Madison, Wis., USA). The RLU was calculated and the results were expressed as percentage of inhibition of STAT3 activity induced by IFN-γ (100% activation) (tables B11.1-B11.3). The experiments for each concentration of the test items were done in triplicate wells.

TABLE B11.1
STAT3 activation by flavonoids and their 5-O-rhamnosides in HeLa cells

| | | | | | | RLU | % |
|---|---|---|---|---|---|---|---|
| | | RLU 1 | RLU 2 | RLU 3 | MEAN | specific | Activation |
| | Control | 2060 | 2067 | 1895 | 2007 | 0 | 0 |
| | IFNγ 25 U/ml | 12482 | 15099 | 15993 | 14525 | 12517 | 100 |
| +IFNγ 25 U/ml | HESR1 25 μM | 13396 | 12243 | 12859 | 12833 | 10825 | 86.48 |
| | HESR1 50 μM | 14303 | 13124 | 11985 | 13137 | 11130 | 88.92 |
| | NR1 25 μM | 10925 | 8301 | 8752 | 9326 | 7319 | 58.47 |
| | NR1 50 μM | 18272 | 6426 | 7599 | 10766 | 8758 | 69.97 |
| | Chrysin 25 μM | 28031 | 22367 | 17504 | 22634 | 20627 | 164.78 |
| | Chrysin 50 μM | 27912 | 3531 | 16304 | 15916 | 13908 | 111.11 |
| | C57dR 25 μM | 11316 | 1954 | 8493 | 7254 | 5247 | 41.92 |
| | C57dR 50 μM | 9196 | 2358 | 6307 | 5954 | 3946 | 31.53 |
| | C5R 25 μM | 7897 | 2398 | 5326 | 5207 | 3200 | 25.56 |
| | C5R 50 μM | 6897 | 7665 | 10507 | 8356 | 6349 | 50.72 |
| | Diosmetin 25 μM | 16337 | 14303 | 17066 | 15902 | 13895 | 111.00 |
| | Diosmetin 50 μM | 9189 | 7751 | 7857 | 8266 | 6258 | 50.00 |
| | D5R 25 μM | 12137 | 10269 | 9275 | 10560 | 8553 | 68.33 |
| | D5R 50 μM | 13005 | 10547 | 10143 | 11232 | 9224 | 73.69 |

TABLE B11.2
STAT3 activation by flavonoids and their 5-O-rhamnosides in HeLa cells

| | | | | | | RLU | % |
|---|---|---|---|---|---|---|---|
| | | RLU 1 | RLU 2 | RLU 3 | MEAN | specific | Activation |
| | Control | 1875 | 1815 | 1815 | 1835 | 0 | 0 |
| | IFNγ 25 U/ml | 9659 | 9851 | 10116 | 9875 | 8040 | 100 |
| +IFNγ 25 U/ml | Biochanin A 25 μM | 9732 | 9023 | 8911 | 9222 | 7387 | 91.87 |
| | Biochanin A 50 μM | 6804 | 12097 | 11786 | 10229 | 8394 | 104.40 |
| | BR1 25 μM | 8162 | 12819 | 11157 | 10713 | 8878 | 110.41 |
| | BR1 50 μM | 12336 | 11620 | 12104 | 12020 | 10185 | 126.67 |
| | BR2 25 μM | 11157 | 10163 | 10660 | 10660 | 8825 | 109.76 |
| | BR2 50 μM | 7983 | 9023 | 11110 | 9372 | 7537 | 93.74 |
| | Silibinin 25 μMl | 12389 | 11170 | 11210 | 11590 | 9755 | 121.32 |
| | Silibinin 50 μM | 12157 | 11885 | 10540 | 11527 | 9692 | 120.55 |

TABLE B11.3
STAT3 activation by flavonoids and their 5-O-rhamnosides in HeLa cells

| | | | | | RLU | % |
|---|---|---|---|---|---|---|
| | RLU 1 | RLU 2 | RLU 3 | MEAN | specific | Activation |
| Control | 2312 | 2233 | 2173 | 2239 | 0 | 0 |
| IFNγ 25 U/ml | 11375 | 10852 | 11269 | 11165 | 9158 | 100 |
| SR1 25 μM + IFNγ | 9507 | 11653 | 10203 | 10454 | 8447 | 92.24 |
| 25 U/ml | | | | | | |
| SR1 50 μM + IFNγ | 10090 | 11355 | 10938 | 10794 | 8787 | 95.95 |
| 25 U/ml | | | | | | |

STAT3 is a transcriptional factor of many genes related to epidermal homeostasis. Its activity has effects on tissue repair and injury healing but also is inhibiting on hair follicle regeneration (Liang (2012) J Neurosci 32:10662-10673). STAT3 activity may even promote melanoma and increases expression of genes linked to cancer and metastasis (Cao (2016) Sci. Rep. 6, 21731).

### Example D-6: Alteration of Glucose Uptake into Cells by Flavonoid 5-O-rhamnosides

Determination of Glucose Uptake in Keratinocytes

HaCaT cells (5×10⁴) were seeded in 96-well black plates and incubated for 24 h. Then, medium was removed and the cells cultivated in OptiMEM, labeled with 50 μM 2-NBDG (2-[N-(7-nitrobenz-2-oxa-1,3-diazol-4-yl) amino]-2-deoxy-D-glucose and treated with the test substances or the positive control, Rosiglitazone, for 24 h. Medium was removed and the wells were carefully washed with PBS and incubated in PBS (100l/well). Finally the fluorescence was measured using the Incucyte FLR software, the data were analyzed by the total green object integrated intensity (GCU×μm2×Well) of the imaging system IncuCyte HD (Essen BioScience). The fluorescence of Rosiglitazone is taken as 100% of glucose uptake, and the glucose uptake was calculated as (% Glucose uptake)=100(T−B)/(R−B), where T (treated) is the fluorescence of test substance-treated cells, B (Basal) is the fluorescence of 2-NBDG cells and P (Positive control) is the fluorescence of cells treated with Rosiglitazone. Results of triplicate measurements are given in tables B12.1 and B12.2.

TABLE B12.1
Influence of flavonoid 5-O-rhamnosides on Glucose uptake in HaCaT cells

| | | Measure | Measure | Measure | | RFU | % Glucose |
|---|---|---|---|---|---|---|---|
| | | 1 | 2 | 3 | Mean | specific | uptake |
| | Control | 8945 | 6910 | 3086 | 6314 | 0 | 0.0 |
| | 2NBDG 50 μM | 176818 | 359765 | 312467 | 283017 | 276703 | 0.0 |
| +2NBDG 50 μM | Rosiglitazone | 776381 | 707003 | 1141924 | 875103 | 868789 | 100.0 |
| | 80 μM | | | | | | |
| | HESR1 25 μM | 756943 | 549324 | 384251 | 563506 | 557192 | 64.1 |
| | HESR1 50 μM | 501977 | 642949 | 529620 | 558182 | 551868 | 63.5 |
| | NR1 25 μM | 493970 | 1160754 | 649291 | 768005 | 761691 | 87.7 |
| | NR1 50 μM | 278134 | 256310 | 257198 | 263881 | 257567 | 29.6 |
| | CR1 25 μM | 291406 | 358114 | 628963 | 426161 | 419847 | 48.3 |
| | CR1 50 μM | 619992 | 595330 | 174412 | 463245 | 456931 | 52.6 |
| | CR2 25 μM | 427937 | 431593 | 390512 | 416681 | 410367 | 47.2 |
| | CR2 50 μM | 771478 | 1100390 | 923151 | 931673 | 925359 | 106.5 |
| | DR2 25 μM | 632398 | 940240 | 197738 | 590125 | 583811 | 67.2 |
| | DR2 50 μM | 2958363 | 1297231 | 2493030 | 2249541 | 2243227 | 258.2 |

1.-54. (canceled)

55. A compound of the following Formula (II) or a solvate thereof 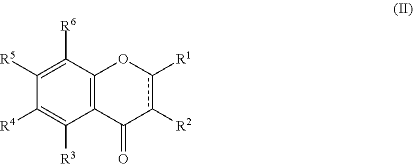
wherein: [symbol]
is a double bond or a single bond;
R¹ and R² are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from —OH; or R¹ and R² are joined together to form, together with the carbon atom(s) that they are attached to, a carbocyclic or heterocyclic ring being optionally substituted with one or more substituents R^{e}; wherein each R^{e} is independently selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
R⁴, R⁵ and R⁶ are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
or alternatively, R⁴ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and
R⁵ and R⁶ are joined together to form, together with the carbon atoms that they are attached to, a carbocyclic or heterocyclic ring being optionally substituted with one or more substituents R^{c};
or alternatively, R⁴ and R⁵ are joined together to form, together with the carbon atoms that they are attached to, a carbocyclic or heterocyclic ring being optionally substituted with one or more substituents R^{c}; and
R⁶ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
each R^{a} is independently selected from a single bond, (C₁₋₅ alkylene, C₂₋₅ alkenylene, arylene and heteroarylene; wherein said alkylene, said alkenylene, said arylene and said heteroarylene are each optionally substituted with one or more groups R^{c};
each R^{b} is independently selected from hydrogen, C₁₋₅ alkyl, C₂₋₅ alkenyl, C₂₋₅ alkynyl, heteroalkyl, cycloalkyl, heterocycloalkyl, aryl and heteroaryl; wherein said alkyl, said alkenyl, said alkynyl, said heteroalkyl, said cycloalkyl, said heterocycloalkyl, said aryl and said heteroaryl are each optionally substituted with one or more groups R^{c};
each R^{c} is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O((C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl;
R³ is —O-(rhamnosyl) wherein said rhamnosyl is optionally substituted at one or more of its —OH groups with one or more groups independently selected from C₁₋₅ alkyl, C₂₋₅ alkenyl, C₂₋₅ alkynyl, a monosaccharide, a disaccharide and an oligosaccharide; and
wherein each R^{d} is independently selected from a monosaccharide, a disaccharide and an oligosaccharide;
with the proviso that, if R⁴ is hydrogen, R⁵ is —OH and [symbol] is a double bond, then R¹ is not methyl.

56. The compound according to claim 55, with the proviso that the compounds naringenin-5-O-α-L-rhamnopyranoside, genistein-5-0-a-L-rhamnopyranoside and eriodictyol-5-0-a-L-rhamnopyranoside are excluded.

57. The compound according to claim 55, wherein each R^{c} is independently selected from halogen, —CF₃, —CN, —OH, —O—R^{d}, —O—C₁₋₄ alkyl, —O-aryl, —S—C₁₋₄ alkyl and —S-aryl.

58. The compound according to claim 55, wherein the compound contains at least one OH group in addition to any OH groups in R³, preferably an OH group directly linked to a carbon atom being linked to a neighboring carbon or nitrogen atom via a double bond.

59. The compound according to claim 55, wherein R⁴, R⁵ and R⁶ are each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O((C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl).

60. The compound according to claim 55, wherein R⁵ is —OH, —O—R^{d} or —O—(C₁₋₅ alkyl).

61. The compound according to claim 55, wherein R⁴ and/or R⁶ is/are hydrogen or —OH.

62. The compound according to claim 55, wherein R³ is —O-α-L-rhamnopyranosyl, —O-α-D-rhamnopyranosyl, —O-β-L-rhamnopyranosyl or —O-β-D-rhamnopyranosyl.

63. The compound according to claim 55, wherein each R^{d} is independently selected from arabinosidyl, galactosidyl, galacturonidyl, mannosidyl, glucosidyl, rhamnosidyl, allosidyl, glucuronidyl, N-acetyl-glucosamidyl, fucosidyl, fucosamidyl, 6-deoxytalosidyl and xylosidyl.

64. The compound according to claim 55, wherein R² is H or —(C₂₋₅ alkenyl).

65. The compound according to claim 55, wherein R¹ and/or R² is/are independently selected from aryl and heteroaryl, wherein said aryl and said heteroaryl are each optionally substituted with one or more groups R^{c}.

66. The compound according to claim 55, wherein R¹ or R² is aryl which is optionally substituted with one or more groups R^{c}, and R² is —H.

67. The compound according to claim 55, wherein R¹ or R² is phenyl, optionally substituted with one, two or three groups independently selected from —OH, —O—R^{d} and —O—C₁₋₄ alkyl.

68. The compound according to claim 55, wherein the compound of formula (II) is a compound of the following formulas (IIa, IIb, IIc) or a solvate thereof: 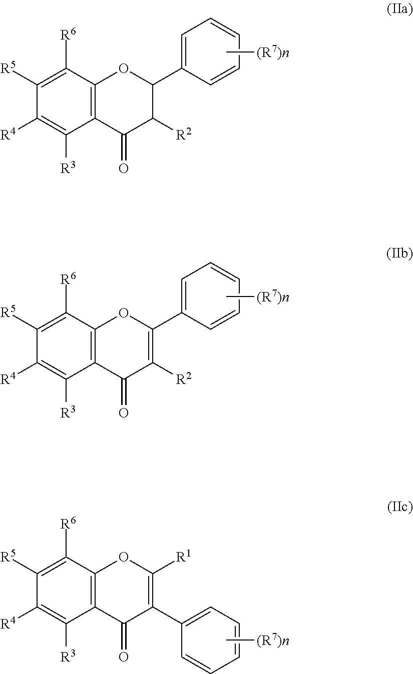
wherein:
R¹, R², R³, R⁴, R⁵ and R⁶ are as defined in claim 55;
each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl;
n is an integer of 0 to 5.

69. The compound according to claim 68, wherein:
R¹ is selected from hydrogen, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};
R² is selected from hydrogen, C₁₋₅ alkyl, C₂₋₅ alkenyl, and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};
R³ is as defined in claim 55,
R⁴ is selected from hydrogen, —OH, —O—R^{d}, C₁₋₅ alkyl, C₂₋₅ alkenyl and —O—(C₁₋₅ alkyl; wherein said alkyl, said alkenyl and the alkyl in said —O—C₁₋₅ alkyl are each optionally substituted with one or more groups independently selected from halogen, —CF₃, —CN, —OH and —O—R^{d};
R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};
R⁶ is selected from hydrogen, —OH, —O—R^{d}, C₁₋₅ alkyl and C₂₋₅ alkenyl, wherein said alkyl and said alkenyl are each optionally substituted with one or more groups R^{c};
each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-((C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—((C₁₋₅ alkyl), —(C₀₋₃ alkylene)-N(C₁₋₅ s alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl;
n is an integer of 0 to 3.

70. The compound according to claim 68, wherein:
R¹ is selected from hydrogen, C₁₋₅ alkyl and C₂₋₅ alkenyl, wherein said alkyl and said alkenyl are each optionally substituted with one or more groups independently selected from halogen, —OH and —O—R^{d};
R² is selected from hydrogen, C₁₋₅ alkyl and C₂₋₅ alkenyl, wherein said alkyl and said alkenyl are each optionally substituted with one or more groups independently selected from halogen, —OH and —O—R^{d};
R³ is as defined in claim 55;
R⁴ is selected from hydrogen, —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d};
R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —OH and —O—R^{d};
each R⁷ is independently selected from C₁₋₅ alkyl, C₂₋₅ alkenyl, —(C₀₋₃ alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH, and —O—R^{d};
n is 0, 1 or 2.

71. The compound according to claim 68, wherein the compound is selected from the following compounds or solvates thereof: 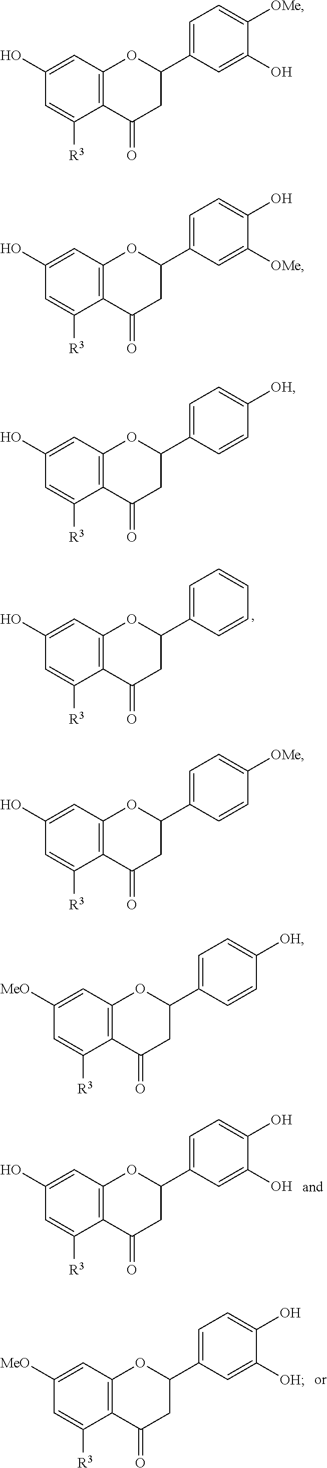 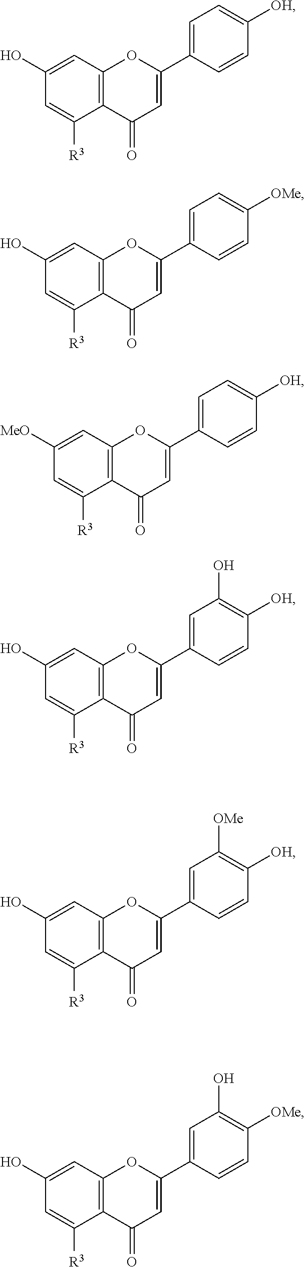 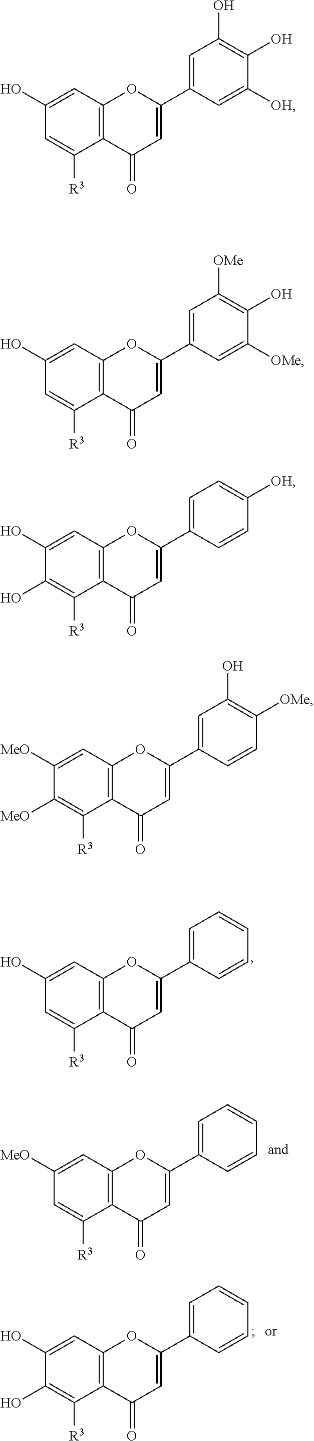 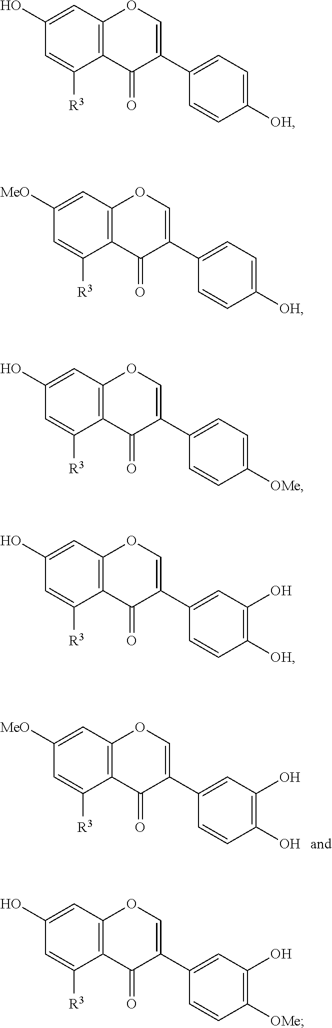
wherein R³ is as defined in claim 55.

72. The compound according to claim 55, wherein the compound of formula (II) is a compound of the following formula (IId) or a solvate thereof: 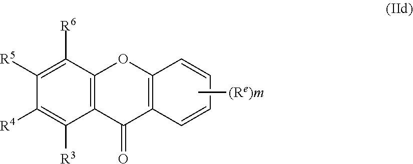
wherein:
R³, R⁴, R⁵, R⁶ and R^{e} are as defined in claim 55; and
m is an integer of 0 to 4.

73. The compound according to claim 72, wherein:
R³ is as defined in claim 55;
R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN—OH and —O—R^{d};
R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};
R⁶ is selected from hydrogen, —OH, —O—R^{d}, C₁₋₅ alkyl and C₂₋₅ alkenyl, wherein said alkyl and said alkenyl are each optionally substituted with one or more groups R^{c};
each R^{e} is independently selected from —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c}; and
m is an integer of 0 to 3.

74. The compound according to claim 72, wherein:
R³ is as defined in claim 55;
R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};
each R^{e} is independently selected from —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d}; and
m is 0, 1 or 2.

75. The compound according to claim 74, wherein the compound is selected from the following compounds or solvates thereof: 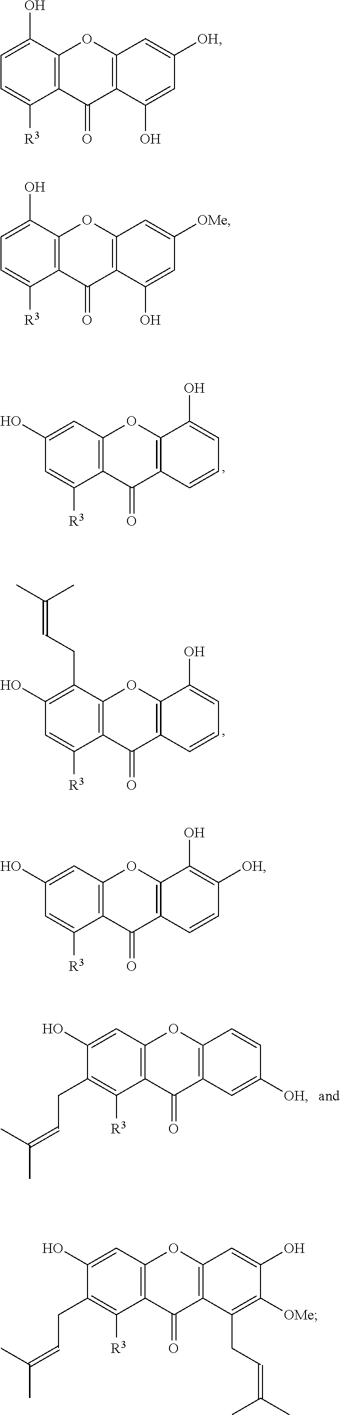
wherein R³ is as defined in claim 55.

76. The compound according to claim 68, wherein R³ is —O-α-L-rhamnopyranosyl, —O-α-D-rhamnopyranosyl, —O-β-L-rhamnopyranosyl or —O-β-D-rhamnopyranosyl.

77. The compound according to claim 68, wherein each R^{d} is independently selected from arabinosidyl, galactosidyl, galacturonidyl, mannosidyl, glucosidyl, rhamnosidyl, allosidyl, glucuronidyl, N-acetyl-glucosamidyl, fucosidyl, fucosamidyl, 6-deoxytalosidyl and xylosidyl.

78. A pharmaceutical composition comprising the compound according to claim 55 and a pharmaceutically acceptable excipient.

79. A method of treating or preventing a disease and/or condition
wherein the method comprises administering to a subject in need thereof, a compound according to claim 55 in a therapeutically effective amount, and
wherein the disease or condition is selected from a skin disease, an allergy, an autoimmune disease, a cardiovascular disease, a lung disease, asthma, a bacterial, viral or parasitic disease, metabolic syndrome, cancer, Alzheimer's disease, arthritis, dysfunctional hair growth, dysfunctional wound healing, or diabetes.

80. A food, drink, animal feed, cosmetic, sun-protectant, flavouring, or dietary supplement comprising a compound according to claim 55.
